(19) United States
(12) Patent Application Publication
(10) Pub. No.: US 2010/0024070 A1
(43) Pub. Date: Jan. 28, 2010

(54) MODULATION OF OIL LEVELS IN PLANTS

(76) Inventors: Steven Craig Bobzin, Malibu, CA (US); Daniel Mumenthaler, Bonita, CA (US); Joel Cruz Rarang, Granada Hills, CA (US)

Correspondence Address: FISH & RICHARDSON P.C., PO BOX 1022, MINNEAPOLIS, MN, 55440-1022, US

(21) Appl. No.: 12/300,833

(22) PCT Filed: May 15, 2007
(86) PCT No.: PCT/US2007/011742

§ 371 Date: Apr. 15, 2009

Related U.S. Application Data
(60) Provisional application No. 60/800,479, filed on May 15, 2006.

(51) Int. Cl. A01H 5/00; C12N 5/04

(52) U.S. Cl. 800/298; 435/419

(57) ABSTRACT

Methods and materials for modulating (e.g., increasing or decreasing) oil levels in plants are disclosed. For example, nucleic acids encoding oil-modulating polypeptides are disclosed as well as methods for using such nucleic acids to transform plant cells. Also disclosed are plants having increased oil levels and plant products produced from plants having increased oil levels.

### TECHNICAL FIELD

This document relates to methods and materials involved in modulating (e.g., increasing or decreasing) oil levels in plants. For example, this document provides plants having increased oil levels as well as materials and methods for making plants and plant products having increased oil levels.

### INCORPORATION-BY-REFERENCE & TEXTS

The material on the accompanying diskette is hereby incorporated by reference into this application. The accompanying compact discs contain one file, 11696-227WO1—Sequence.txt, which was created on May 14, 2007. The file named 11696-227WO1—Sequence.txt is 934 KB. The file can be accessed using Microsoft Word on a computer that uses Windows OS.

### BACKGROUND

Fat, protein, and carbohydrates are nutrients that supply calories to the body. Fat provides nine calories per gram, which is more than twice the number provided by carbohydrates or protein. Dietary fats are composed of fatty acids and glycerol. The glycerol can be converted to glucose by the liver and used as a source of energy. The fatty acids are a good source of energy for many tissues, especially heart and skeletal muscle.

Fatty acids consist of carbon chains of various lengths and a terminal carboxylic acid group. Saturated fatty acids do not contain any double bonds or other functional groups along the chain. A saturated fatty acid has the maximum possible number of hydrogen atoms attached to every carbon atom. Therefore, it is said to be saturated with hydrogen atoms. Eating too much saturated fat is one of the major risk factors for heart disease. Saturated fats are found in animal products such as butter, cheese, whole milk, ice cream, cream, and fatty meats. Saturated fats are also found in some vegetable oils, such as coconut, palm, and palm kernel oils. Most other vegetable oils contain unsaturated fat that helps to lower blood cholesterol if used in place of saturated fat.

Unsaturated fatty acids contain one or more double bonds between carbon atoms and, therefore, two fewer hydrogen atoms per double bond. A fatty acid with a single double bond is called a monounsaturated fatty acid. A fatty acid with two or more double bonds is called a polyunsaturated fatty acid. Polyunsaturated fats are liquid at room temperature, and remain in liquid form even when refrigerated or frozen. Polyunsaturated fats are divided into two families: the omega-3 fats and the omega-6 fats.

The omega-3 family of fatty acids includes alpha-linolenic acid (ALA). ALA is an essential fatty acid that cannot be synthesized in the body and must, therefore, be consumed in the diet. Dietary sources of ALA include canola, flaxseed, flaxseed oil, soybean, and pumpkin seed oil. Omega-3 fatty acids have been found to reduce the risks of heart problems, lower high blood pressure, and ameliorate autoimmune diseases.

Omega-6 fatty acids are beneficial as well. The omega-6 family of fatty acids includes linoleic acid, which is another essential fatty acid. The body converts linoleic acid to gamma linoleic acid (GLA) and ultimately to prostaglandins, which are hormone-like molecules that help regulate inflammation and blood pressure as well as heart, gastrointestinal, and kidney functions. The main sources of omega-6 fatty acids are vegetable oils such as corn oil and soy oil.

Vegetable oil is fat extracted from plant sources. Vegetable oils are used in cooking, in making margarine and other processed foods, and in producing several non-food items such as soap, cosmetics, medicine, and paint. Since vegetable oils are usually extracted from the seeds of the plant, seed oil yield has a significant impact on the economics of producing many products. Increasing seed oil content may increase the economic return per unit to the seller of the seed in addition to increasing the nutritional value to the consumer of the seed.

### SUMMARY

This document provides methods and materials related to plants having modulated (e.g., increased or decreased) levels of oil. For example, this document provides transgenic plants and plant cells having increased levels of oil, nucleic acids used to generate transgenic plants and plant cells having increased levels of oil, and methods for making plants and plant cells having increased levels of oil. Such plants and plant cells can be grown to produce, for example, seeds having increased oil content. Increasing the oil content of seeds can increase the nutritional value of the seeds and the yield of oil obtained from the seeds, which may benefit both food consumers and producers.

In one aspect, a method of modulating the level of oil in a plant in provided. The method comprises introducing into a plant cell an isolated nucleic acid comprising a nucleotide sequence encoding a polypeptide having 80 percent or greater sequence identity to an amino acid sequence selected from the group consisting of SEQ ID NO:80, SEQ ID NOs:82-85, SEQ ID NOs:87-127, SEQ ID NOs:129-130, SEQ ID NOs:132-133, SEQ ID NOs:135-138, SEQ ID NOs:140-146, SEQ ID NOs:148-149, SEQ ID NO:151, SEQ ID NO:153, SEQ ID NOs:155-160, SEQ ID NOs:162-164, SEQ ID NO:166, SEQ ID NOs:168-171, SEQ ID NO:173, SEQ ID NOs:175-177, SEQ ID NOs:179-183, SEQ ID NOs:185-188, SEQ ID NOs:190-193, SEQ ID NOs:195-196, SEQ ID NOs:198-199, SEQ ID NO:201, SEQ ID NOs:203-214, SEQ ID NO:216, SEQ ID NO:218, SEQ ID NOs:220-227, SEQ ID NOs:229-230, SEQ ID NOs:232-235, SEQ ID NOs:237-243, SEQ ID NO:245, and SEQ ID NOs:247-249, where a tissue of a plant produced from the plant cell has a difference in the level of oil as compared to the corresponding level in tissue of a control plant that does not comprise the nucleic acid.

In another aspect, a method of modulating the level of oil in a plant is provided. The method comprises introducing into a plant cell an isolated nucleic acid comprising a nucleotide sequence encoding a polypeptide having 80 percent or greater sequence identity to an amino acid sequence selected from the group consisting of SEQ ID NO:80, SEQ ID NO:82, SEQ ID NO:87, SEQ ID NOs:89-90, SEQ ID NO:129, SEQ ID NO:132, SEQ ID NO:135, SEQ ID NO:140, SEQ ID NO:143, SEQ ID NOs:148-149, SEQ ID NO:151, SEQ ID NO: 153, SEQ ID NOs:155-159, SEQ ID NOs:162-164, SEQ ID NO:166, SEQ ID NOs:168-171, SEQ ID NO:173, SEQ ID NOs:175-177, SEQ ID NOs:179-181, SEQ ID NO:183, SEQ ID NOs:185-186, SEQ ID NOs:190-192, SEQ ID NOs:195-196, SEQ ID NOs:198-199, SEQ ID NO:201, SEQ ID NOs:203-206, SEQ ID NOs:208-214, SEQ ID NO:216, SEQ ID NO:218, SEQ ID NO:220, SEQ ID NOs:222-226, SEQ ID NOs:229-230, SEQ ID NOs:232-233, SEQ ID NO:235, SEQ ID NO:237, SEQ ID NO:242, SEQ ID NO:245, SEQ ID NO:247, and SEQ ID NO:249, where a tissue of a plant produced from the plant cell has a difference in the level of oil as compared to the corresponding level in tissue of a control plant that does not comprise the nucleic acid.

In another aspect, a method of modulating the level of oil in a plant is provided. The method comprises introducing into a plant cell an isolated nucleic acid comprising a nucleotide sequence encoding a polypeptide having 80 percent or greater sequence identity to an amino acid sequence selected from the group consisting of SEQ ID NO:80, SEQ ID NO:82, SEQ ID NO:87, SEQ ID NOs:89-90, SEQ ID NO:129, SEQ ID NO:132, SEQ ID NO:135, SEQ ID NO:140, SEQ ID NO:143, SEQ ID NOs:148-149, SEQ ID NO:151, SEQ ID NO:153, SEQ ID NOs:155-159, SEQ ID NOs:162-164, SEQ ID NO:166, SEQ ID NOs:168-171, SEQ ID NO:173, SEQ ID NOs:175-177, SEQ ID NOs:179-181, SEQ ID NO:183, SEQ ID NOs:185-186, SEQ ID NOs:190-192, SEQ ID NOs:195-196, SEQ ID NOs:198-199, SEQ ID NO:201, SEQ ID NOs:203-206, SEQ ID NOs:208-214, SEQ ID NO:216, SEQ ID NO:218, SEQ ID NO:220, SEQ ID NOs:222-226, SEQ ID NOs:229-230, SEQ ID NOs:232-233, SEQ ID NO:235, SEQ ID NO:237, SEQ ID NO:242, SEQ ID NO:245, SEQ ID NO:247, and SEQ ID NO:249, where a tissue of a plant produced from the plant cell has a difference in the level of oil as compared to the corresponding level in tissue of a control plant that does not comprise the nucleic acid.

The sequence identity can be 85 percent or greater, 90 percent or greater, or 95 percent or greater. The nucleotide sequence can encode a polypeptide comprising an amino acid sequence corresponding SEQ ID NO:80. The nucleotide sequence can encode a polypeptide comprising an amino acid sequence corresponding to SEQ ID NO:82. The nucleotide sequence can encode a polypeptide comprising an amino acid sequence corresponding to SEQ ID NO:87. The nucleotide sequence can encode a polypeptide comprising an amino acid sequence corresponding to SEQ ID NO:148. The nucleotide sequence can encode a polypeptide comprising an amino acid sequence corresponding to SEQ ID NO:151. The nucleotide sequence can encode a polypeptide comprising an amino acid sequence corresponding to SEQ ID NO:162. The nucleotide sequence can encode a polypeptide comprising an amino acid sequence corresponding to SEQ ID NO:173. The nucleotide sequence can encode a polypeptide comprising an amino acid sequence corresponding to SEQ ID NO:175. The nucleotide sequence can encode a polypeptide comprising an amino acid sequence corresponding to SEQ ID NO:185. The nucleotide sequence can encode a polypeptide comprising an amino acid sequence corresponding to SEQ ID NO:190. The nucleotide sequence can encode a polypeptide comprising an amino acid sequence corresponding to SEQ ID NO:198. The nucleotide sequence can encode a polypeptide comprising an amino acid sequence corresponding to SEQ ID NO:201. The nucleotide sequence can encode a polypeptide comprising an amino acid sequence corresponding to SEQ ID NO:203. The nucleotide sequence can encode a polypeptide comprising an amino acid sequence corresponding to SEQ ID NO:216. The nucleotide sequence can encode a polypeptide comprising an amino acid sequence corresponding to SEQ ID NO:229. The nucleotide sequence can encode a polypeptide comprising an amino acid sequence corresponding to SEQ ID NO:245. The difference can be an increase in the level of oil. The isolated nucleic acid can be operably linked to a regulatory region. The regulatory region can be a promoter. The promoter can be a tissue-preferential, broadly expressing, or inducible promoter. The plant can be a dicot. The plant can be a member of the genus Anacardium, Arachis, Azadirachta, Brassica, Cannabis, Carthamus, Corylus, Crambe, Cucurbita, Glycine, Gossypium, Helianthus, Jatropha, Juglans, Linum, Olea, Papaver, Persea, Prunus, Ricinus, Sesamum, Simmondsia, or Vitis. The plant can be a monocot. The plant can be a member of the genus Cocos, Elaeis, Oryza, or Zea. The tissue can be seed tissue.

A method of producing a plant tissue is also provided. The method comprises growing a plant cell comprising an exogenous nucleic acid comprising a nucleotide sequence encoding a polypeptide having 80 percent or greater sequence identity to an amino acid sequence selected from the group consisting of SEQ ID NO:80, SEQ ID NOs:82-85, SEQ ID NOs:87-127, SEQ ID NOs:129-130, SEQ ID NOs:132-133, SEQ ID NOs:135-138, SEQ ID NOs:140-146, SEQ ID NOs:148-149, SEQ ID NO:151, SEQ ID NO:153, SEQ ID NOs:155-160, SEQ ID NOs:162-164, SEQ ID NO:166, SEQ ID NOs:168-171, SEQ ID NO:173, SEQ ID NOs:175-177, SEQ ID NOs:179-183, SEQ ID NOs:185-188, SEQ ID NOs:190-193, SEQ ID NOs:195-196, SEQ ID NOs:198-199, SEQ ID NO:201, SEQ ID NOs:203-214, SEQ ID NO:216, SEQ ID NO:218, SEQ ID NOs:220-227, SEQ ID NOs:229-230, SEQ ID NOs:232-235, SEQ ID NOs:237-243, SEQ ID NO:245, and SEQ ID NOs:247-249, where the tissue has a difference in the level of oil as compared to the corresponding level in tissue of a control plant that does not comprise the nucleic acid.

In another aspect, a method of producing a plant tissue is provided. The method comprises growing a plant cell comprising an exogenous nucleic acid comprising a nucleotide sequence encoding a polypeptide having 80 percent or greater sequence identity to an amino acid sequence selected from the group consisting of SEQ ID NO:80, SEQ ID NO:82, SEQ ID NO:87, SEQ ID NOs:89-90, SEQ ID NO:129, SEQ ID NO:132, SEQ ID NO:135, SEQ ID NO:140, SEQ ID NO:143, SEQ ID NOs:148-149, SEQ ID NO:151, SEQ ID NO:153, SEQ ID NOs:155-159, SEQ ID NOs:162-164, SEQ ID NO:166, SEQ ID NOs:168-171, SEQ ID NO:173, SEQ ID NOs:175-177, SEQ ID NOs:179-181, SEQ ID NO:183, SEQ ID NOs:185-186, SEQ ID NOs:190-192, SEQ ID NOs:195-196, SEQ ID NOs:198-199, SEQ ID NO:201, SEQ ID NOs:203-206, SEQ ID NOs:208-214, SEQ ID NO:216, SEQ ID NO:218, SEQ ID NO:220, SEQ ID NOs:222-226, SEQ ID NOs:229-230, SEQ ID NOs:232-233, SEQ ID NO:235, SEQ ID NO:237, SEQ ID NO:242, SEQ ID NO:245, SEQ ID NO:247, and SEQ ID NO:249, where the tissue has a difference in the level of oil as compared to the corresponding level in tissue of a control plant that does not comprise the nucleic acid.

In another aspect, a method of producing a plant tissue is provided. The method comprises growing a plant cell comprising an exogenous nucleic acid comprising a nucleotide sequence encoding a polypeptide having 80 percent or greater sequence identity to an amino acid sequence selected from the group consisting of SEQ ID NO:80, SEQ ID NO:82, SEQ ID NO:87, SEQ ID NOs:89-90, SEQ ID NO:129, SEQ ID NO:132, SEQ ID NO:135, SEQ ID NO:140, SEQ ID NO:143, SEQ ID NOs:148-149, SEQ ID NO:151, SEQ ID NO:153, SEQ ID NOs:155-159, SEQ ID NOs:162-164, SEQ ID NO:166, SEQ ID NOs:168-171, SEQ ID NO:173, SEQ ID NOs:175-177, SEQ ID NOs:179-181, SEQ ID NO:183, SEQ ID NOs:185-186, SEQ ID NOs:190-192, SEQ ID NOs:195-196, SEQ ID NOs:198-199, SEQ ID NO:201, SEQ ID NOs:203-206, SEQ ID NOs:208-214, SEQ ID NO:216, SEQ ID NO:218, SEQ ID NO:220, SEQ ID NOs:222-226, SEQ ID NOs:229-230, SEQ ID NOs:232-233, SEQ ID NO:235, SEQ ID NO:237, SEQ ID NO:242, SEQ ID NO:245, SEQ ID NO:247, and SEQ ID NO:249, where the tissue has a difference in the level of oil as compared to the corresponding level in tissue of a control plant that does not comprise the nucleic acid.

The sequence identity can be 85 percent or greater, 90 percent or greater, or 95 percent or greater. The nucleotide sequence can encode a polypeptide comprising an amino acid sequence corresponding to SEQ ID NO:80. The nucleotide sequence can encode a polypeptide comprising an amino acid sequence corresponding to SEQ ID NO:82. The nucleotide sequence can encode a polypeptide comprising an amino acid sequence corresponding to SEQ ID NO:87. The nucleotide sequence can encode a polypeptide comprising an amino acid sequence corresponding to SEQ ID NO:148. The nucleotide sequence can encode a polypeptide comprising an amino acid sequence corresponding to SEQ ID NO:151. The nucleotide sequence can encode a polypeptide comprising an amino acid sequence corresponding to SEQ ID NO:162. The nucleotide sequence can encode a polypeptide comprising an amino acid sequence corresponding to SEQ ID NO:173. The nucleotide sequence can encode a polypeptide comprising an amino acid sequence corresponding to SEQ ID NO:175. The nucleotide sequence can encode a polypeptide comprising an amino acid sequence corresponding to SEQ ID NO:185. The nucleotide sequence can encode a polypeptide comprising an amino acid sequence corresponding to SEQ ID NO:190. The nucleotide sequence can encode a polypeptide comprising an amino acid sequence corresponding to SEQ ID NO:198. The nucleotide sequence can encode a polypeptide comprising an amino acid sequence corresponding to SEQ ID NO:201. The nucleotide sequence can encode a polypeptide comprising an amino acid sequence corresponding to SEQ ID NO:203. The nucleotide sequence can encode a polypeptide comprising an amino acid sequence corresponding to SEQ ID NO:216. The nucleotide sequence can encode a polypeptide comprising an amino acid sequence corresponding to SEQ ID NO:229. The nucleotide sequence can encode a polypeptide comprising an amino acid sequence corresponding to SEQ ID NO:245. The difference can be an increase in the level of oil. The exogenous nucleic acid can be operably linked to a regulatory region. The regulatory region can be a promoter. The promoter can be a tissue-preferential, broadly expressing, or inducible promoter. The plant tissue can be dicotyledonous. The plant tissue can be a member of the genus Anacardium, Arachis, Azadirachta, Brassica, Cannabis, Carthamus, Corylus, Crambe, Cucurbita, Glycine, Gossypium, Helianthus, Jatropha, Juglans, Linum, Olea, Papaver, Persea, Prunus, Ricinus, Sesamum, Simmondsia, or Vitis. The plant tissue can be monocotyledonous. The plant tissue can be a member of the genus Cocos, Elaeis, Oryza, or Zea. The tissue can be seed tissue.

A plant cell is also provided. The plant cell comprises an exogenous nucleic acid comprising a nucleotide sequence encoding a polypeptide having 80 percent or greater sequence identity to an amino acid sequence selected from the group consisting of SEQ ID NO:80, SEQ ID NOs:82-85, SEQ ID NOs:87-127, SEQ ID NOs:129-130, SEQ ID NOs:132-133, SEQ ED NOs:135-138, SEQ ID NOs:140-146, SEQ ID NOs:148-149, SEQ ID NO:151, SEQ ID NO:153, SEQ ID NOs:155-160, SEQ ID NOs:162-164, SEQ ID NO:166, SEQ ID NOs:168-171, SEQ ID NO:173, SEQ ID NOs:175-177, SEQ ID NOs:179-183, SEQ ID NOs:185-188, SEQ ID NOs:190-193, SEQ ID NOs:195-196, SEQ ID NOs:198-199, SEQ ID NO:201, SEQ ID NOs:203-214, SEQ ID NO:216, SEQ ID NO:218, SEQ ID NOs:220-227, SEQ ID NOs:229-230, SEQ ID NOs:232-235, SEQ ID NOs:237-243, SEQ ID NO:245, and SEQ ID NOs:247-249, where a tissue of a plant produced from the plant cell has a difference in the level of oil as compared to the corresponding level in tissue of a control plant that does not comprise the nucleic acid.

In another aspect, a plant cell is provided. The plant cell comprises an exogenous nucleic acid comprising a nucleotide sequence encoding a polypeptide having 80 percent or greater sequence identity to an amino acid sequence selected from the group consisting of SEQ ID NO:80, SEQ ID NO:82, SEQ ID NO:87, SEQ ID NOs:89-90, SEQ ID NO:129, SEQ ID NO:132, SEQ ID NO:135, SEQ ID NO:140, SEQ ID NO:143, SEQ ID NOs:148-149, SEQ ID NO:151, SEQ ID NO:153, SEQ ID NOs:155-159, SEQ ID NOs:162-164, SEQ ID NO:166, SEQ ID NOs:168-171, SEQ ID NO:173, SEQ ID NOs:175-177, SEQ ID NOs:179-181, SEQ ID NO:183, SEQ ID NOs:185-186, SEQ ID NOs:190-192, SEQ ID NOs:195-196, SEQ ID NOs:198-199, SEQ ID NO:201, SEQ ID NOs:203-206, SEQ ID NOs:208-214, SEQ ID NO:216, SEQ ID NO:218, SEQ ID NO:220, SEQ ID NOs:222-226, SEQ ID NOs:229-230, SEQ ID NOs:232-233, SEQ ID NO:235, SEQ ID NO:237, SEQ ID NO:242, SEQ ID NO:245, SEQ ID NO:247, and SEQ ID NO:249, where a tissue of a plant produced from the plant cell has a difference in the level of oil as compared to the corresponding level in tissue of a control plant that does not comprise the nucleic acid.

In another aspect, a plant cell is provided. The plant cell comprises an exogenous nucleic acid comprising a nucleotide sequence encoding a polypeptide having 80 percent or greater sequence identity to an amino acid sequence selected from the group consisting of SEQ ID NO:80, SEQ ID NO:82, SEQ ID NO:87, SEQ ID NOs:89-90, SEQ ID NO:129, SEQ ID NO:132, SEQ ID NO:135, SEQ ID NO:140, SEQ ID NO:143, SEQ ID NOs:148-149, SEQ ID NO:151, SEQ ID NO:153, SEQ ID NOs:155-159, SEQ ID NOs:162-164, SEQ ID NO:166, SEQ ID NOs:168-171, SEQ ID NO:173, SEQ ID NOs:175-177, SEQ ID NOs:179-181, SEQ ID NO:183, SEQ ID NOs:185-186, SEQ ID NOs:190-192, SEQ ID NOs:195-196, SEQ ID NOs:198-199, SEQ ID NO:201, SEQ ID NOs:203-206, SEQ ID NOs:208-214, SEQ ID NO:216, SEQ ID NO:218, SEQ ID NO:220, SEQ ID NOs:222-226, SEQ ID NOs:229-230, SEQ ID NOs:232-233, SEQ ID NO:235, SEQ ID NO:237, SEQ ID NO:242, SEQ ID NO:245, SEQ ID NO:247, and SEQ ID NO:249, where a tissue of a plant produced from the plant cell has a difference in the level of oil as compared to the corresponding level in tissue of a control plant that does not comprise the nucleic acid.

The sequence identity can 85 percent or greater, 90 percent or greater, or 95 percent or greater. The nucleotide sequence can encode a polypeptide comprising an amino acid sequence corresponding to SEQ ID NO:80. The nucleotide sequence can encode a polypeptide comprising an amino acid sequence corresponding to SEQ ID NO:82. The nucleotide sequence can encode a polypeptide comprising an amino acid sequence corresponding to SEQ ID NO:87. The nucleotide sequence can encode a polypeptide comprising an amino acid sequence corresponding to SEQ ID NO:148. The nucleotide sequence can encode a polypeptide comprising an amino acid sequence corresponding to SEQ ID NO:151. The nucleotide sequence can encode a polypeptide comprising an amino acid sequence corresponding to SEQ ID NO:162. The nucleotide sequence can encode a polypeptide comprising an amino acid sequence corresponding to SEQ ID NO:173. The nucleotide sequence can encode a polypeptide comprising an amino acid sequence corresponding to SEQ ID NO:175. The nucleotide sequence can encode a polypeptide comprising an amino acid sequence corresponding to SEQ ID NO:185. The nucleotide sequence can encode a polypeptide comprising an amino acid sequence corresponding to SEQ ID NO:190. The nucleotide sequence can encode a polypeptide comprising an amino acid sequence corresponding to SEQ ID NO:198. The nucleotide sequence can encode a polypeptide comprising an amino acid sequence corresponding to SEQ ID NO:201. The nucleotide sequence can encode a polypeptide comprising an amino acid sequence corresponding to SEQ ID NO:203. The nucleotide sequence can encode a polypeptide comprising an amino acid sequence corresponding to SEQ ID NO:216. The nucleotide sequence can encode a polypeptide comprising an amino acid sequence corresponding to SEQ ID NO:229. The nucleotide sequence can encode a polypeptide comprising an amino acid sequence corresponding to SEQ ID NO:245. The difference can be an increase in the level of oil. The exogenous nucleic acid can be operably linked to a regulatory region. The regulatory region can be a promoter. The promoter can be a tissue-preferential, broadly expressing, or inducible promoter. The plant can be a dicot. The plant can be a member of the genus Anacardium, Arachis, Azadirachta, Brassica, Cannabis, Carthamus, Corylus, Crambe, Cucurbita, Glycine, Gossypium, Helianthus, Jatropha, Juglans, Linum, Olea, Papaver, Persea, Prunus, Ricinus, Sesamum, Simmondsia, or Vitis. The plant can be a monocot. The plant can be a member of the genus Cocos, Elaeis, Oryza, or Zea. The tissue can be seed tissue.

A transgenic plant is also provided. The transgenic plant comprises any of the plant cells described above. Progeny of the transgenic plant are also provided. The progeny has a difference in the level of oil as compared to the level of oil in a corresponding control plant that does not comprise the exogenous nucleic acid. Seed, vegetative tissue, and fruit from the transgenic plant are also provided. In addition, food products and feed products comprising seed, vegetative tissue, or fruit from the transgenic plant are provided. Oil from the seed of the transgenic plant is provided, as is a method of making oil. The method comprises extracting oil from the seed of the transgenic plant.

In another aspect, a method of modulating the level of oil in a plant is provided. The method comprises introducing into a plant cell an exogenous nucleic acid comprising a nucleotide sequence encoding a polypeptide, where the HMM bit score of the amino acid sequence of the polypeptide is greater than 50, the HMM based on the amino acid sequences depicted in one of FIGS. 1-14, and where a tissue of a plant produced from the plant cell has a difference in the level of oil as compared to the corresponding level in tissue of a control plant that does not comprise the exogenous nucleic acid.

In another aspect, a method of modulating the level of oil in a plant is provided. The method comprises introducing into a plant cell an exogenous nucleic acid comprising a nucleotide sequence encoding a polypeptide 50-85 amino acids in length, where the polypeptide is the amino terminus of a polypeptide having at least 450 amino acids and having an HMM bit score greater than 622, the HMM based on the amino acid sequences depicted in FIG. 15, and where a tissue of a plant produced from the plant cell has a difference in the level of oil as compared to the corresponding level in tissue of a control plant that does not comprise the exogenous nucleic acid.

In another aspect, a method of modulating the level of oil in a plant is provided. The method comprises introducing into a plant cell an exogenous nucleic acid comprising a nucleotide sequence encoding a polypeptide having 80 percent or greater sequence identity to an amino acid sequence selected from the group consisting of SEQ ID NO:80, SEQ ID NOs:82-85, SEQ ID NOs:87-127, SEQ ID NOs:129-130, SEQ ID NOs:132-133, SEQ ID NOs:135-138, SEQ ID NOs:140-146, SEQ ID NOs:148-149, SEQ ID NO:151, SEQ ID NO:153, SEQ ID NOs:155-160, SEQ ID NOs:162-164, SEQ ID NO:166, SEQ ID NOs:168-171, SEQ ID NO:173, SEQ ID NOs:175-177, SEQ ID NOs:179-183, SEQ ID NOs:185-188, SEQ ID NOs:190-193, SEQ ID NOs:195-196, SEQ ID NOs:198-199, SEQ ID NO:201, SEQ ID NOs:203-214, SEQ ID NO:216, SEQ ID NO:218, SEQ ID NOs:220-227, SEQ ID NOs:229-230, SEQ ID NOs:232-235, SEQ ID NOs:237-243, SEQ ID NO:245, SEQ ID NOs:247-249, SEQ ID NO:309, SEQ ID NO:311, SEQ ID NO:313, SEQ ID NO:315, SEQ ID NOs:317-318, SEQ ID NOs:320-321, SEQ ID NO:323, SEQ ID NO:325, SEQ ID NO:327, SEQ ID NO:329, SEQ ID NOs:331-332, SEQ ID NOs:334-335, SEQ ID NOs:337-338, SEQ ID NOs:340-341, SEQ ID NOs:343-344, SEQ ID NO:346, SEQ ID NO:348, SEQ ID NO:350, SEQ ID NOs:352-353, SEQ ID NO:355, SEQ ID NO:357, SEQ ID NOs:359-360, SEQ ID NO:367, and SEQ ID NOs:415-441, where a tissue of a plant produced from the plant cell has a difference in the level of oil as compared to the corresponding level in tissue of a control plant that does not comprise the exogenous nucleic acid. The nucleotide sequence can encode a polypeptide comprising an amino acid sequence corresponding to SEQ ID NO:148.

In another aspect, a method of modulating the level of oil in a plant is provided. The method comprises introducing into a plant cell an exogenous nucleic acid comprising a nucleotide sequence having 80 percent or greater sequence identity to a nucleotide sequence selected from the group consisting of SEQ ID NO:79, SEQ ID NO:81, SEQ ID NO:86, SEQ ID NO:128, SEQ ID NO:131, SEQ ID NO:134, SEQ ID NO:139, SEQ ID NO:147, SEQ ID NO:150, SEQ ID NO:152, SEQ ID NO:154, SEQ ID NO:161, SEQ ID NO:165, SEQ ID NO:167, SEQ ID NO:172, SEQ ID NO:174, SEQ ID NO:178, SEQ ID NO:184, SEQ ID NO:189, SEQ ID NO:194, SEQ ID NO:197, SEQ ID NO:200, SEQ ID NO:202, SEQ ID NO:215, SEQ ID NO:217, SEQ ID NO:219, SEQ ID NO:228, SEQ ID NO:231, SEQ ID NO:236, SEQ ID NO:244, SEQ ID NO:246, SEQ ID NOs:265-308, SEQ ID NO:310, SEQ ID NO:312, SEQ ID NO:314, SEQ ID NO:316, SEQ ID NO:319, SEQ ID NO:322, SEQ ID NO:324, SEQ ID NO:326, SEQ ID NO:328, SEQ ID NO:330, SEQ ID NO:333, SEQ ID NO:336, SEQ ID NO:339, SEQ ID NO:342, SEQ ID NO:345, SEQ ID NO:347, SEQ ID NO:349, SEQ ID NO:351, SEQ ID NO:354, SEQ ID NO:356, SEQ ID NO:358, and SEQ ID NOs:361-366, where a tissue of a plant produced from the plant cell has a difference in the level of oil as compared to the corresponding level in tissue of a control plant that does not comprise the exogenous nucleic acid. The nucleotide sequence can comprise the nucleotide sequence set forth in SEQ ID NO:147.

The difference can be an increase in the level of oil. The exogenous nucleic acid can be operably linked to a regulatory region.

In another aspect, a method of modulating the level of oil in a plant is provided. The method comprises introducing into a plant cell an exogenous nucleic acid comprising a regulatory region operably linked to a polynucleotide whose transcription product is at least 30 nucleotides in length and is complementary to a nucleic acid encoding a polypeptide, where the HMM bit score of the amino acid sequence of the polypeptide is greater than 50, the HMM based on the amino acid sequences depicted in one of FIGS. 1-14, where the regulatory region modulates transcription of the polynucleotide in the plant cell, and where a tissue of a plant produced from the plant cell has a difference in the level of oil as compared to the corresponding level in tissue of a control plant that does not comprise the exogenous nucleic acid. The HMM bit score can be 100 or greater.

In another aspect, a method of modulating the level of oil in a plant is provided. The method comprises introducing into a plant cell an exogenous nucleic acid comprising a regulatory region operably linked to a polynucleotide that is transcribed into an interfering RNA effective for inhibiting expression of a polypeptide having 80 percent or greater sequence identity to an amino acid sequence selected from the group consisting of SEQ ID NO:80, SEQ ID NOs:82-85, SEQ ID NOs:87-127, SEQ ID NOs:129-130, SEQ ID NOs:132-133, SEQ BD NOs:135-138, SEQ ID NOs:140-146, SEQ ID NOs:148-149, SEQ ID NO:151, SEQ ID NO:153, SEQ ID NOs:155-160, SEQ ID NOs:162-164, SEQ ID NO:166, SEQ ID NOs:168-171, SEQ ID NO:173, SEQ ID NOs:175-177, SEQ ID NOs:179-183, SEQ ID NOs:185-188, SEQ ID NOs:190-193, SEQ ID NOs:195-196, SEQ ID NOs:198-199, SEQ ID NO:201, SEQ ID NOs:203-214, SEQ ID NO:216, SEQ ID NO:218, SEQ ID NOs:220-227, SEQ ID NOs:229-230, SEQ ID NOs:232-235, SEQ ID NOs:237-243, SEQ ID NO:245, SEQ ID NOs:247-249, SEQ ID NO:309, SEQ ID NO:311, SEQ ID NO:313, SEQ ID NO:315, SEQ ID NOs:317-318, SEQ ID NOs:320-321, SEQ ID NO:323, SEQ ID NO:325, SEQ ID NO:327, SEQ ID NO:329, SEQ ID NOs:331-332, SEQ ID NOs:334-335, SEQ ID NOs:337-338, SEQ ID NOs:340-341, SEQ ID NOs:343-344, SEQ ID NO:346, SEQ ID NO:348, SEQ ID NO:350, SEQ ID NOs:352-353, SEQ ID NO:355, SEQ ID NO:357, SEQ ID NOs:359-360, SEQ ID NO:367, and SEQ ID NOs:415-441, where the regulatory region modulates transcription of the polynucleotide in the plant cell, and where a tissue of a plant produced from the plant cell has a difference in the level of oil as compared to the corresponding level in tissue of a control plant that does not comprise the exogenous nucleic acid. The exogenous nucleic acid can further comprise a 3′ UTR operably linked to the polynucleotide. The polynucleotide can be transcribed into an interfering RNA comprising a stem-loop structure. The stem-loop structure can comprise an inverted repeat of the 3′ UTR.

The difference can be a decrease in the level of oil. The sequence identity can be 85 percent or greater, 90 percent or greater, or 95 percent or greater. The method can further comprise the step of producing a plant from the plant cell. The introducing step can comprise introducing the nucleic acid into a plurality of plant cells. The method can further comprise the step of producing a plurality of plants from the plant cells. The method can further comprise the step of selecting one or more plants from the plurality of plants that have the difference in the level of oil. The regulatory region can be a tissue-preferential, broadly expressing, or inducible promoter.

In another aspect, a method of producing a plant tissue is provided. The method can comprise growing a plant cell comprising an exogenous nucleic acid comprising a nucleotide sequence encoding a polypeptide, where the HMM bit score of the amino acid sequence of the polypeptide is greater than 50, the HMM based on the amino acid sequences depicted in one of FIGS. 1-14, and where the tissue has a difference in the level of oil as compared to the corresponding level in tissue of a control plant that does not comprise the exogenous nucleic acid.

In another aspect, a method of producing a plant tissue is provided. The method comprises growing a plant cell comprising an exogenous nucleic acid comprising a nucleotide sequence encoding a polypeptide 50-85 amino acids in length, where the polypeptide is the amino terminus of a polypeptide having at least 450 amino acids and having an HMM bit score greater than 622, the HMM based on the amino acid sequences depicted in FIG. 15, and where the tissue has a difference in the level of oil as compared to the corresponding level in tissue of a control plant that does not comprise the nucleic acid.

In another aspect, a method of producing a plant tissue is provided. The method comprises growing a plant cell comprising an exogenous nucleic acid comprising a nucleotide sequence encoding a polypeptide having 80 percent or greater sequence identity to an amino acid sequence selected from the group consisting of SEQ ID NO:80, SEQ ID NOs:82-85, SEQ ID NOs:87-127, SEQ ID NOs:129-130, SEQ ID NOs:132-133, SEQ ID NOs:135-138, SEQ ID NOs:140-146, SEQ ID NOs:148-149, SEQ ID NO:151, SEQ ID NO:153, SEQ ID NOs:155-160, SEQ ID NOs:162-164, SEQ ID NO:166, SEQ ID NOs:168-171, SEQ ID NO:173, SEQ ID NOs:175-177, SEQ ID NOs:179-183, SEQ ID NOs:185-188, SEQ ID NOs:190-193, SEQ ID NOs:195-196, SEQ ID NOs:198-199, SEQ ID NO:201, SEQ ID NOs:203-214, SEQ ID NO:216, SEQ ID NO:218, SEQ ID NOs:220-227, SEQ ID NOs:229-230, SEQ ID NOs:232-235, SEQ ID NOs:237-243, SEQ ID NO:245, SEQ ID NOs:247-249, SEQ ID NO:309, SEQ ID NO:311, SEQ ID NO:313, SEQ ID NO:315, SEQ ID NOs:317-318, SEQ ID NOs:320-321, SEQ ID NO:323, SEQ ID NO:325, SEQ ID NO:327, SEQ ID NO:329, SEQ ID NOs:331-332, SEQ ID NOs:334-335, SEQ ID NOs:337-338, SEQ ID NOs:340-341, SEQ ID NOs:343-344, SEQ ID NO:346, SEQ ID NO:348, SEQ ID NO:350, SEQ ID NOs:352-353, SEQ ID NO:355, SEQ ID NO:357, SEQ ID NOs:359-360, SEQ ID NO:367, and SEQ ID NOs:415-441, where the tissue has a difference in the level of oil as compared to the corresponding level in tissue of a control plant that does not comprise the nucleic acid.

In another aspect, a method of producing a plant tissue is provided. The method comprises growing a plant cell comprising an exogenous nucleic acid comprising a nucleotide sequence having 80 percent or greater sequence identity to a nucleotide sequence selected from the group consisting of SEQ ID NO:79, SEQ ID NO:81, SEQ ID NO:86, SEQ ID NO:128, SEQ ID NO:131, SEQ ID NO:134, SEQ ID NO:139, SEQ ID NO:147, SEQ ID NO:150, SEQ ID NO:152, SEQ ID NO:154, SEQ ID NO:161, SEQ ID NO:165, SEQ ID NO:167, SEQ ID NO:172, SEQ ID NO:174, SEQ ID NO:178, SEQ ID NO:184, SEQ ID NO:189, SEQ ID NO:194, SEQ ID NO:197, SEQ ID NO:200, SEQ ID NO:202, SEQ ID NO:215, SEQ ID NO:217, SEQ ID NO:219, SEQ ID NO:228, SEQ ID NO:231, SEQ ID NO-236, SEQ ID NO:244, SEQ ID NO:246, SEQ ID NOs:265-308, SEQ ID NO:310, SEQ ID NO:312, SEQ ID NO:314, SEQ ID NO:316, SEQ ID NO:319, SEQ ID NO:322, SEQ ID NO:324, SEQ ID NO:326, SEQ ID NO:328, SEQ ID NO:330, SEQ ID NO:333, SEQ ID NO:336, SEQ ID NO:339, SEQ ID NO:342, SEQ ID NO:345, SEQ ID NO:347, SEQ ID NO:349, SEQ ID NO:351, SEQ ID NO:354, SEQ ID NO:356, SEQ ID NO:358, and SEQ ID NOs:361-366, where the tissue has a difference in the level of oil as compared to the corresponding level in tissue of a control plant that does not comprise the nucleic acid.

In another aspect, a method of producing a plant tissue is provided. The method comprises growing a plant cell comprising an exogenous nucleic acid comprising a regulatory region operably linked to a polynucleotide whose transcription product is at least 30 nucleotides in length and is complementary to a nucleic acid encoding a polypeptide, where the HMM bit score of the amino acid sequence of the polypeptide is greater than 50, the HMM based on the amino acid sequences depicted in one of FIGS. 1-14, where the regulatory region modulates transcription of the polynucleotide in the plant cell, and where the tissue has a difference in the level of oil as compared to the corresponding level in tissue of a control plant that does not comprise the nucleic acid.

In another aspect, a method of producing a plant tissue is provided. The method comprises growing a plant cell comprising an exogenous nucleic acid comprising a regulatory region operably linked to a polynucleotide that is transcribed into an interfering RNA effective for inhibiting expression of a polypeptide having 80 percent or greater sequence identity to an amino acid sequence selected from the group consisting of SEQ ID NO:80, SEQ ID NOs:82-85, SEQ ID NOs:87-127, SEQ ID NOs:129-130, SEQ ID NOs:132-133, SEQ ID NOs:135-138, SEQ ID NOs:140-146, SEQ ID NOs:148-149, SEQ ID NO:151, SEQ ID NO:153, SEQ ID NOs:155-160, SEQ ID NOs:162-164, SEQ ID NO:166, SEQ ID NOs:168-171, SEQ ID NO:173, SEQ ID NOs:175-177, SEQ ID NOs:179-183, SEQ ID NOs:185-188, SEQ ID NOs:190-193, SEQ ID NOs:195-196, SEQ ID NOs:198-199, SEQ ID NO:201, SEQ ID NOs:203-214, SEQ ID NO:216, SEQ ID NO:218, SEQ ID NOs:220-227, SEQ ID NOs:229-230, SEQ ID NOs:232-235, SEQ ID NOs:237-243, SEQ ID NO:245, SEQ ID NOs:247-249, SEQ ID NO:309, SEQ ID NO:311, SEQ ID NO:313, SEQ ID NO:315, SEQ ID NOs:317-318, SEQ ID NOs:320-321, SEQ ID NO:323, SEQ ID NO:325, SEQ ID NO:327, SEQ ID NO:329, SEQ ID NOs:331-332, SEQ ID NOs:334-335, SEQ ID NOs:337-338, SEQ ID NOs:340-341, SEQ ID NOs:343-344, SEQ ID NO:346, SEQ ID NO:348, SEQ ID NO:350, SEQ ID NOs:352-353, SEQ ID NO:355, SEQ ID NO:357, SEQ ID NOs:359-360, SEQ ID NO:367, and SEQ ID NOs:415-441, where the regulatory region modulates transcription of the polynucleotide in the plant cell, and where the tissue has a difference in the level of oil as compared to the corresponding level in tissue of a control plant that does not comprise the nucleic acid.

The plant can be a dicot. The plant can be a member of the genus Anacardium, Arachis, Azadirachta, Brassica, Cannabis, Carthamus, Corylus, Crambe, Cucurbita, Glycine, Gossypium, Helianthus, Jatropha, Juglans, Linum, Olea, Papaver, Persea, Prunus, Ricinus, Sesamum, Simmondsia, or Vitis. The plant can be a monocot. The plant can be a member of the genus Cocos, Elaeis, Oryza, or Zea. The plant can be a species selected from the group consisting of Miscanthus hybrid (Miscanthus×giganteus), Miscanthus sinensis, Miscanthus sacchariflorus, Panicum virgatum, Populus balsamifera, Sorghum bicolor, and Saccharum spp. The tissue can be seed tissue.

In another aspect, a plant cell comprising an exogenous nucleic acid is provided. The exogenous nucleic acid comprises a nucleotide sequence encoding a polypeptide, where the HMM bit score of the amino acid sequence of the polypeptide is greater than 50, the HMM based on the amino acid sequences depicted in one of FIGS. 1-14, and where a tissue of a plant produced from the plant cell has a difference in the level of oil as compared to the corresponding level in tissue of a control plant that does not comprise the nucleic acid.

In another aspect, a plant cell comprising an exogenous nucleic acid is provided. The exogenous nucleic acid comprises a nucleotide sequence encoding a polypeptide 50-85 amino acids in length, where the polypeptide is the amino terminus of a polypeptide having at least 450 amino acids and having an HMM bit score greater than 622, the HMM based on the amino acid sequences depicted in FIG. 15, and where a tissue of a plant produced from the plant cell has a difference in the level of oil as compared to the corresponding level in tissue of a control plant that does not comprise the nucleic acid.

In another aspect, a plant cell comprising an exogenous nucleic acid is provided. The exogenous nucleic acid comprises a nucleotide sequence encoding a polypeptide having 80 percent or greater sequence identity to an amino acid sequence selected from the group consisting of SEQ ID NO:80, SEQ ID NOs:82-85, SEQ ID NOs:87-127, SEQ ID NOs:129-130, SEQ ID NOs:132-133, SEQ ID NOs:135-138, SEQ ID NOs:140-146, SEQ ID NOs:148-149, SEQ ID NO:151, SEQ ID NO:153, SEQ ID NOs:155-160, SEQ ID NOs:162-164, SEQ ID NO:166, SEQ ID NOs:168-171, SEQ ID NO:173, SEQ ID NOs:175-177, SEQ ID NOs:179-183, SEQ ID NOs:185-188, SEQ ID NOs:190-193, SEQ ID NOs:195-196, SEQ ID NOs:198-199, SEQ ID NO:201, SEQ ID NOs:203-214, SEQ ID NO:216, SEQ ID NO:218, SEQ ID NOs:220-227, SEQ ID NOs:229-230, SEQ ID NOs:232-235, SEQ ID NOs:237-243, SEQ ID NO:245, SEQ ID NOs:247-249, SEQ ID NO:309, SEQ ID NO:311, SEQ ID NO:313, SEQ ID NO:315, SEQ ID NOs:317-318, SEQ ID NOs:320-321, SEQ ID NO:323, SEQ ID NO:325, SEQ ID NO:327, SEQ ID NO:329, SEQ ID NOs:331-332, SEQ ID NOs:334-335, SEQ ID NOs:337-338, SEQ ID NOs:340-341, SEQ ID NOs:343-344, SEQ ID NO:346, SEQ ID NO:348, SEQ ID NO:350, SEQ ID NOs:352-353, SEQ ID NO:355, SEQ ID NO:357, SEQ ID NOs:359-360, SEQ ID NO:367, and SEQ ID NOs:415-441, where a tissue of a plant produced from the plant cell has a difference in the level of oil as compared to the corresponding level in tissue of a control plant that does not comprise the nucleic acid.

In another aspect, a plant cell comprising an exogenous nucleic acid is provided. The exogenous nucleic acid comprises a nucleotide sequence having 80 percent or greater sequence identity to a nucleotide sequence selected from the group consisting of SEQ ID NO:79, SEQ ID NO:81, SEQ ID NO:86, SEQ ID NO:128, SEQ ID NO:131, SEQ ID NO:134, SEQ ID NO:139, SEQ ID NO:147, SEQ ID NO:150, SEQ ID NO:152, SEQ ID NO:154, SEQ ID NO:161, SEQ ID NO:165, SEQ ID NO:167, SEQ ID NO:172, SEQ ID NO:174, SEQ ID NO:178, SEQ ID NO:184, SEQ ID NO:189, SEQ ID NO:194, SEQ ID NO:197, SEQ ID NO:200, SEQ ID NO:202, SEQ ID NO:215, SEQ ID NO:217, SEQ ID NO:219, SEQ ID NO:228, SEQ ID NO:231, SEQ ID NO:236, SEQ ID NO:244, SEQ ID NO:246, SEQ ID NOs:265-308, SEQ ID NO:310, SEQ ID NO:312, SEQ ID NO:314, SEQ ID NO:316, SEQ ID NO:319, SEQ ID NO:322, SEQ ID NO:324, SEQ ID NO:326, SEQ ID NO:328, SEQ ID NO:330, SEQ ID NO:333, SEQ ID NO:336, SEQ ID NO:339, SEQ ID NO:342, SEQ ID NO:345, SEQ ID NO:347, SEQ ID NO:349, SEQ ID NO:351, SEQ ID NO:354, SEQ ID NO:356, SEQ ID NO:358, and SEQ ID NOs:361-366, where a tissue of a plant produced from the plant cell has a difference in the level of oil as compared to the corresponding level in tissue of a control plant that does not comprise the nucleic acid.

In another aspect, a plant cell comprising an exogenous nucleic acid is provided. The exogenous nucleic acid comprises a regulatory region operably linked to a polynucleotide whose transcription product is at least 30 nucleotides in length and is complementary to a nucleic acid encoding a polypeptide, where the HMM bit score of the amino acid sequence of the polypeptide is greater than 50, the HMM based on the amino acid sequences depicted in one of FIGS. 1-14, where the regulatory region modulates transcription of the polynucleotide in the plant cell, and where a tissue of a plant produced from the plant cell has a difference in the level of oil as compared to the corresponding level in tissue of a control plant that does not comprise the nucleic acid.

In another aspect, a plant cell comprising an exogenous nucleic acid is provided. The exogenous nucleic acid comprises a regulatory region operably linked to a polynucleotide that is transcribed into an interfering RNA effective for inhibiting expression of a polypeptide having 80 percent or greater sequence identity to an amino acid sequence selected from the group consisting of SEQ ID NO:80, SEQ ID NOs:82-85, SEQ ID NOs:87-127, SEQ ID NOs:129-130, SEQ ID NOs:132-133, SEQ ID NOs:135-138, SEQ ID NOs:140-146, SEQ ID NOs:148-149, SEQ ID NO:151, SEQ ID NO:153, SEQ ID NOs:155-160, SEQ ID NOs:162-164, SEQ ID NO:166, SEQ ID NOs:168-171, SEQ ID NO:173, SEQ ID NOs:175-177, SEQ ID NOs:179-183, SEQ ID NOs:185-188, SEQ ID NOs:190-193, SEQ ID NOs:195-196, SEQ ID NOs:198-199, SEQ ID NO:201, SEQ ID NOs:203-214, SEQ ID NO:216, SEQ ID NO:218, SEQ ID NOs:220-227, SEQ ID NOs:229-230, SEQ ID NOs:232-235, SEQ ID NOs:237-243, SEQ ID NO:245, SEQ ID NOs:247-249, SEQ ID NO:309, SEQ ID NO:311, SEQ ID NO:313, SEQ ID NO:315, SEQ ID NOs:317-318, SEQ ID NOs:320-321, SEQ ID NO:323, SEQ ID NO:325, SEQ ID NO:327, SEQ ID NO:329, SEQ ID NOs:331-332, SEQ ID NOs:334-335, SEQ ID NOs:337-338, SEQ ID NOs:340-341, SEQ ID NOs:343-344, SEQ ID NO:346, SEQ ID NO:348, SEQ ID NO:350, SEQ ID NOs:352-353, SEQ ID NO:355, SEQ ID NO:357, SEQ ID NOs:359-360, SEQ ID NO:367, and SEQ ID NOs:415-441, where the regulatory region modulates transcription of the polynucleotide in the plant cell, and where a tissue of a plant produced from the plant cell has a difference in the level of oil as compared to the corresponding level in tissue of a control plant that does not comprise the nucleic acid.

The plant can be a dicot. The plant can be a member of the genus Anacardium, Arachis, Azadirachta, Brassica, Cannabis, Carthamus, Corylus, Crambe, Cucurbita, Glycine, Gossypium, Helianthus, Jatropha, Juglans, Linum, Olea, Papaver, Persea, Prunus, Ricinus, Sesamum, Simm ondsia, or Vitis. The plant can be a monocot. The plant can be a member of the genus Cocos, Elaeis, Oryza, or Zea. The plant can be a species selected from the group consisting of Miscanthus hybrid (Miscanthus×giganteus), Miscanthus sinensis, Miscanthus sacchariflorus, Panicum virgatum, Populus balsamifera, Sorghum bicolor, and Saccharum spp. The tissue can be seed tissue.

In another aspect, a transgenic plant is provided. The transgenic plant comprises any of the plant cells described above. Progeny of the transgenic plant are also provided. The progeny has a difference in the level of oil as compared to the level of oil in a corresponding control plant that does not comprise the exogenous nucleic acid. Seed, vegetative tissue, and fruit from the transgenic plant are also provided, as is a method of making oil. The method comprises extracting oil from the seed of the transgenic plant.

In another aspect, an isolated nucleic acid is provided. The isolated nucleic acid comprises a nucleotide sequence having 95% or greater sequence identity to a nucleotide sequence selected from the group consisting of SEQ ID NO:79, SEQ ID NO:81, SEQ ID NO:128, SEQ ID NO:131, SEQ ID NO:134, SEQ ID NO:139, SEQ ID NO:152, SEQ ID NO:154, SEQ ID NO:165, SEQ ID NO:167, SEQ ID NO:178, SEQ ID NO:194, SEQ ID NO:217, SEQ ID NO:219, SEQ ID NO:231, SEQ ID NO:236, SEQ ID NO:246, SEQ ID NOs:265-308, SEQ ID NO:310, SEQ ID NO:312, SEQ ID NO:314, SEQ ID NO:316, SEQ ID NO:319, SEQ ID NO:322, SEQ ID NO:324, SEQ ID NO:326, SEQ ID NO:328, SEQ ID NO:330, SEQ ID NO:333, SEQ ID NO:336, SEQ ID NO:339, SEQ ID NO:342, SEQ ID NO:345, SEQ ID NO:347, SEQ ID NO:349, SEQ ID NO:351, SEQ ID NO:354, SEQ ID NO:356, SEQ ID NO:358, SEQ ID NO:366, SEQ ID NO:368, SEQ ID NO:370, SEQ ID NO:372, SEQ ID NO:374, SEQ ID NO:376, SEQ ID NO:378, SEQ ID NO:380, and SEQ ID NO:413.

In another aspect, an isolated nucleic acid is provided. The isolated nucleic acid comprises a nucleotide sequence encoding a polypeptide having 80% or greater sequence identity to an amino acid sequence selected from the group consisting of SEQ ID NO:80, SEQ ID NO:82, SEQ ID NO:89, SEQ ID NO:90, SEQ ID NO:129, SEQ ID NO:132, SEQ ID NO:135, SEQ ID NO:140, SEQ ID NO:143, SEQ ID NO:149, SEQ ID NO:153, SEQ ID NOs:155-159, SEQ ID NOs:163-164, SEQ ID NO:166, SEQ ID NOs:168-171, SEQ ID NOs:176-177, SEQ ID NOs:179-181, SEQ ID NO:183, SEQ ID NO:186, SEQ ID NOs:191-192, SEQ ID NOs:195-196, SEQ ID NO:199, SEQ ID NOs:204-206, SEQ ID NOs:208-213, SEQ ID NO:218, SEQ ID NO:220, SEQ ID NOs:222-226, SEQ ID NO:230, SEQ ID NOs:232-233, SEQ ID NO:235, SEQ ID NO:237, SEQ ID NO:242, SEQ ID NO:247, SEQ ID NO:249, SEQ ID NO:309, SEQ ID NO:311, SEQ ID NO:313, SEQ ID NO:315, SEQ ID NO:317, SEQ ID NO:320, SEQ ID NO:323, SEQ ID NO:325, SEQ ID NO:327, SEQ ID NO:329, SEQ ID NO:331, SEQ ID NO:334, SEQ ID NO:337, SEQ ID NO:340, SEQ ID NO:343, SEQ ID NO:346, SEQ ID NO:348, SEQ ID NO:350, SEQ ID NO:352, SEQ ID NO:355, SEQ ID NO:357, SEQ ID NO:359, SEQ ID NO:367, SEQ ID NO:369, SEQ ID NO:371, SEQ ID NO:373, SEQ ID NO:375, SEQ ID NO:377, SEQ ID NO:379, SEQ ID NO:381, and SEQ ID NO:414.

In another aspect, a method of modulating the level of oleic acid in a plant is provided. The method comprises introducing into a plant cell an exogenous nucleic acid comprising a nucleotide sequence encoding a polypeptide, where the HMM bit score of the amino acid sequence of the polypeptide is greater than 235, the HMM based on the amino acid sequences depicted in FIG. 3, and where a tissue of a plant produced from the plant cell has a difference in the level of oleic acid as compared to the corresponding level in tissue of a control plant that does not comprise the exogenous nucleic acid.

In another aspect, a method of modulating the level of oleic acid in a plant is provided. The method comprises introducing into a plant cell an exogenous nucleic acid comprising a nucleotide-sequence encoding a polypeptide having 80 percent or greater sequence identity to the amino acid sequence set forth in SEQ ID NO:148, where a tissue of a plant produced from the plant cell has a difference in the level of oleic acid as compared to the corresponding level in tissue of a control plant that does not comprise the exogenous nucleic acid.

In another aspect, a method of modulating the level of oleic acid in a plant is provided. The method comprises introducing into a plant cell an exogenous nucleic acid comprising a nucleotide sequence having 80 percent or greater sequence identity to the nucleotide sequence set forth in SEQ ID NO:147, where a tissue of a plant produced from the plant cell has a difference in the level of oleic acid as compared to the corresponding level in tissue of a control plant that does not comprise the exogenous nucleic acid.

The difference can be an increase in the level of oleic acid. The exogenous nucleic acid can be operably linked to a regulatory region.

In another aspect, a method of modulating the level of oleic acid in a plant is provided. The method comprises introducing into a plant cell an exogenous nucleic acid comprising a regulatory region operably linked to a polynucleotide whose transcription product is at least 30 nucleotides in length and is complementary to a nucleic acid encoding a polypeptide, where the HMM bit score of the amino acid sequence of the polypeptide is greater than 235, the HMM based on the amino acid sequences depicted in FIG. 3, where the regulatory region modulates transcription of the polynucleotide in the plant cell, and where a tissue of a plant produced from the plant cell has a difference in the level of oleic acid as compared to the corresponding level in tissue of a control plant that does not comprise the exogenous nucleic acid.

In another aspect, a method of modulating the level of oleic acid in a plant is provided. The method comprises introducing into a plant cell an exogenous nucleic acid comprising a regulatory region operably linked to a polynucleotide that is transcribed into an interfering RNA effective for inhibiting expression of a polypeptide having 80 percent or greater sequence identity to the amino acid sequence set forth in SEQ ID NO:148, where the regulatory region modulates transcription of the polynucleotide in the plant cell, and where a tissue of a plant produced from the plant cell has a difference in the level of oleic acid as compared to the corresponding level in tissue of a control plant that does not comprise the exogenous nucleic acid. The exogenous nucleic acid can further comprise a 3′ UTR operably linked to the polynucleotide. The polynucleotide can be transcribed into an interfering RNA comprising a stem-loop structure. The stem-loop structure can comprise an inverted repeat of the 3′ UTR.

The difference can be a decrease in the level of oleic acid. The sequence identity can be 85 percent or greater, 90 percent or greater, or 95 percent or greater. The method can further comprise the step of producing a plant from the plant cell. The introducing step can comprise introducing the nucleic acid into a plurality of plant cells. The method can further comprise the step of producing a plurality of plants from the plant cells. The method can further comprise the step of selecting one or more plants from the plurality of plants that have the difference in the level of oleic acid. The regulatory region can be a tissue-preferential, broadly expressing, or inducible promoter.

In another aspect, a method of producing a plant tissue is provided. The method comprises growing a plant cell comprising an exogenous nucleic acid comprising a nucleotide sequence encoding a polypeptide, where the HMM bit score of the amino acid sequence of the polypeptide is greater than 235, the HMM based on the amino acid sequences depicted in FIG. 3, and where the tissue has a difference in the level of oleic acid as compared to the corresponding level in tissue of a control plant that does not comprise the exogenous nucleic acid.

In another aspect, a method of producing a plant tissue is provided. The method comprises growing a plant cell comprising an exogenous nucleic acid comprising a nucleotide sequence encoding a polypeptide having 80 percent or greater sequence identity to the amino acid sequence set forth in SEQ ID NO:148, where the tissue has a difference in the level of oleic acid as compared to the corresponding level in tissue of a control plant that does not comprise the nucleic acid.

In another aspect, a method of producing a plant tissue is provided. The method comprises growing a plant cell comprising an exogenous nucleic acid comprising a nucleotide sequence having 80 percent or greater sequence identity to the nucleotide sequence set forth in SEQ ID NO:147, where the tissue has a difference in the level of oleic acid as compared to the corresponding level in tissue of a control plant that does not comprise the nucleic acid.

In another aspect, a method of producing a plant tissue is provided. The method comprises growing a plant cell comprising an exogenous nucleic acid comprising a regulatory region operably linked to a polynucleotide whose transcription product is at least 30 nucleotides in length and is complementary to a nucleic acid encoding a polypeptide, where the HMM bit score of the amino acid sequence of the polypeptide is greater than 235, the HMM based on the amino acid sequences depicted in FIG. 3, where the regulatory region modulates transcription of the polynucleotide in the plant cell, and where the tissue has a difference in the level of oleic acid as compared to the corresponding level in tissue of a control plant that does not comprise the nucleic acid.

In another aspect, a method of producing a plant tissue is provided. The method comprises growing a plant cell comprising an exogenous nucleic acid comprising a regulatory region operably linked to a polynucleotide that is transcribed into an interfering RNA effective for inhibiting expression of a polypeptide having 80 percent or greater sequence identity to the amino acid sequence set forth in SEQ ID NO:148, where the regulatory region modulates transcription of the polynucleotide in the plant cell, and where the tissue has a difference in the level of oleic acid as compared to the corresponding level in tissue of a control plant that does not comprise the nucleic acid.

The plant can be a dicot. The plant can be a member of the genus Anacardium, Arachis, Azadirachta, Brassica, Cannabis, Carthamus, Corylus, Crambe, Cucurbita, Glycine, Gossypium, Helianthus, Jatropha, Juglans, Linum, Olea, Papaver, Persea, Prunus, Ricinus, Sesamum, Simmondsia, or Vitis. The plant can be a monocot. The plant can be a member of the genus Cocos, Elaeis, Oryza, or Zea. The plant can be a species selected from the group consisting of Miscanthus hybrid (Miscanthus×giganteus), Miscanthus sinensis, Miscanthus sacchariflorus, Panicum virgatum, Populus balsamifera, Sorghum bicolor, and Saccharum spp. The tissue can be seed tissue.

In another aspect, a plant cell comprising an exogenous nucleic acid is provided. The exogenous nucleic acid comprises a nucleotide sequence encoding a polypeptide, where the HMM bit score of the amino acid sequence of the polypeptide is greater than 235, the HMM based on the amino acid sequences depicted in FIG. 3, and where a tissue of a plant produced from the plant cell has a difference in the level of oleic acid as compared to the corresponding level in tissue of a control plant that does not comprise the nucleic acid.

In another aspect, a plant cell comprising an exogenous nucleic acid is provided. The exogenous nucleic acid comprises a nucleotide sequence encoding a polypeptide having 80 percent or greater sequence identity to the amino acid sequence set forth in SEQ ID NO:148, where a tissue of a plant produced from the plant cell has a difference in the level of oleic acid as compared to the corresponding level in tissue of a control plant that does not comprise the nucleic acid.

In another aspect, a plant cell comprising an exogenous nucleic acid is provided. The exogenous nucleic acid comprises a nucleotide sequence having 80 percent or greater sequence identity to the nucleotide sequence set forth in SEQ ID NO:147, where a tissue of a plant produced from the plant cell has a difference in the level of oleic acid as compared to the corresponding level in tissue of a control plant that does not comprise the nucleic acid.

In another aspect, a plant cell comprising an exogenous nucleic acid is provided. The exogenous nucleic acid comprises a regulatory region operably linked to a polynucleotide whose transcription product is at least 30 nucleotides in length and is complementary to a nucleic acid encoding a polypeptide, where the HMM bit score of the amino acid sequence of the polypeptide is greater than 235, the HMM based on the amino acid sequences depicted in FIG. 3, where the regulatory region modulates transcription of the polynucleotide in the plant cell, and where a tissue of a plant produced from the plant cell has a difference in the level of oleic acid as compared to the corresponding level in tissue of a control plant that does not comprise the nucleic acid.

In another aspect, a plant cell comprising an exogenous nucleic acid is provided. The exogenous nucleic acid comprises a regulatory region operably linked to a polynucleotide that is transcribed into an interfering RNA effective for inhibiting expression of a polypeptide having 80 percent or greater sequence identity to the amino acid sequence set forth in SEQ ID NO:148, where the regulatory region modulates transcription of the polynucleotide in the plant cell, and where a tissue of a plant produced from the plant cell has a difference in the level of oleic acid as compared to the corresponding level in tissue of a control plant that does not comprise the nucleic acid.

The plant can be a dicot. The plant can be a member of the genus Anacardium, Arachis, Azadirachta, Brassica, Cannabis, Carthamus, Corylus, Crambe, Cucurbita, Glycine, Gossypium, Helianthus, Jatropha, Juglans, Linum, Olea, Papaver, Persea, Prunus, Ricinus, Sesamum, Simmondsia, or Vitis. The plant can be a monocot. The plant can be a member of the genus Cocos, Elaeis, Oryza, or Zea. The plant can be a species selected from the group consisting of Miscanthus hybrid (Miscanthus×giganteus), Miscanthus sinensis, Miscanthus sacchariflorus, Panicum virgatum, Populus balsamifera, Sorghum bicolor, and Saccharum spp. The tissue can be seed tissue.

In another aspect, a transgenic plant is provided. The transgenic plant comprises any of the plant cells described above. Progeny of the transgenic plant are also provided. The progeny has a difference in the level of oleic acid as compared to the level of oleic acid in a corresponding control plant that does not comprise the exogenous nucleic acid. Seed, vegetative tissue, and fruit from the transgenic plant are also provided, as is a method of making oil. The method comprises extracting oil from the seed of the transgenic plant.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is an alignment of Clone 625035 (SEQ ID NO:82) with homologous and/or orthologous amino acid sequences gi|32401273 (SEQ ID NO:83), gi|14140141 (SEQ ID NO:84), and Clone 1926437 (SEQ ID NO:343). FIG. 1 and the other alignment figures provided herein were generated using the program MUSCLE version 3.52 (Edgar, Nucleic Acids Res, 32(5):1792-97 (2004); World Wide Web at drive5.com/muscle).

FIG. 2 is an alignment of Clone 5344 (SEQ ID NO:87) with homologous and/or orthologous amino acid sequences gi|26094811 (SEQ ID NO:88), Clone 1411115 (SEQ ID NO:90), gi|3337095 (SEQ ID NO:91), gi|3337091 (SEQ ID NO:92), gi|18148925 (SEQ ID NO:93), gi|1617034 (SEQ ID NO:95), gi|3205177 (SEQ ID NO:96), gi|33469566 (SEQ ID NO:98), gi|3978580 (SEQ ID NO:101), gi|3978578 (SEQ ID NO:102), gi|19110472 (SEQ ID NO:103), gi|19110474 (SEQ ID NO:104), gi|9110478 (SEQ ID NO:105), gi|17221624 (SEQ ID NO:106), gi|58379364 (SEQ ID NO:109), gi|19110476 (SEQ ID NO:110), gi|1143381 (SEQ ID NO:111), gi|34068091 (SEQ ID NO:112), gi|54306529 (SEQ ID NO:114), gi|8778050 (SEQ ID NO:117), gi|57868641 (SEQ ID NO:118), gi|76365455 (SEQ ID NO:119), gi|33087508 (SEQ ID NO:120), gi|38234920 (SEQ ID NO:121), gi|33087506 (SEQ ID NO:122), gi|33087512 (SEQ ID NO:124), gi|40732890 (SEQ ID NO:126), gi|2460188 (SEQ ID NO:127), Annot 1534757 (SEQ ID NO:129), gi|1679733 (SEQ ID NO:130), gi|21667647 (SEQ ID NO:133), gi|469457 (SEQ ID NO:136), gi|13172312 (SEQ ID NO:137), gi|30984105 (SEQ ID NO:138), gi|20066308 (SEQ ID NO:141), gi|444011 (SEQ ID NO:142), Clone 784385 (SEQ ID NO:143), gi|55859509 (SEQ ID NO:144), and Clone 571162 (SEQ ID NO:249).

FIG. 3 is an alignment of Annot 828248_T (SEQ ID NO:360) with homologous and/or orthologous amino acid sequence Clone 948978 (SEQ ID NO:149).

FIG. 4 is an alignment of Annot 569483 (SEQ ID NO:151) with homologous and/or orthologous amino acid sequences Annot 1488415 (SEQ ID NO:153), Clone 524650 (SEQ ID NO:156), Clone 237720 (SEQ ID NO:157), Clone 703914 (SEQ ID NO:159), and gi|50881429 (SEQ ID NO:160).

FIG. 5 is an alignment of Annot 565281 (SEQ ID NO:162) with homologous and/or orthologous amino acid sequences Clone 952316 (SEQ ID NO:163), Clone 649261 (SEQ ID NO:164), Annot 1469350 (SEQ ID NO:166), Clone 234461 (SEQ ID NO:169), and Clone 1327188 (SEQ ID NO:171).

FIG. 6 is an alignment of Annot 542494 (SEQ ID NO:175) with homologous and/or orthologous amino acid sequences Clone 1369396 (SEQ ID NO:176), Clone 1102549 (SEQ ID NO:177), Annot 1515577 (SEQ ID NO:179), Clone 516401 (SEQ ID NO:180), Clone 618542 (SEQ ID NO:181), and gi|50940451 (SEQ ID NO:182).

FIG. 7 is an alignment of Annot 549258 (SEQ ID NO:185) with homologous and/or orthologous amino acid sequences Clone 945519 (SEQ ID NO:186) and gi|50935585 (SEQ ID NO:187).

FIG. 8 is an alignment of Annot 564261 (SEQ ID NO:190) with homologous and/or orthologous amino acid sequences Clone 947761 (SEQ ID NO:191), Clone 680759 (SEQ ID NO:192), gi|77549263 (SEQ ID NO:193), Annot 1486789 (SEQ ID NO:195), Clone 230678 (SEQ ID NO:196), Clone 1715450 (SEQ ID NO:311), Clone 1849790 (SEQ ID NO:315), and Clone 1795526 (SEQ ID NO:317).

FIG. 9 is an alignment of Annot 565548 (SEQ ID NO:198) with homologous and/or orthologous amino acid sequence Clone 976147 (SEQ ID NO:199).

FIG. 10 is an alignment of Clone 2721 (SEQ ID NO:203) with homologous and/or orthologous amino acid sequences Clone 871180 (SEQ ID NO:204), Clone 1767185 (SEQ ID NO:206), gi|1617213 (SEQ ID NO:207), Clone 772741 (SEQ ID NO:208), gi|1617206 (SEQ ID NO:318), Clone 1808894 (SEQ ID NO:320), and gi|1617197 (SEQ ID NO:321).

FIG. 11 is an alignment of Clone 30018 (SEQ ID NO:216) with homologous and/or orthologous amino acid sequences Annot 1488347 (SEQ ID NO:218), gi|633685 (SEQ ID NO:221), Clone 853331 (SEQ ID NO:222), Clone 208991 (SEQ ID NO:223), Clone 639802 (SEQ ID NO:226), gi|4775284 (SEQ ID NO:227), Clone 959117 (SEQ ID NO:323), Clone 1797853 (SEQ ID NO:329), Clone 1620853 (SEQ ID NO:331), gi|92867670 (SEQ ID NO:332), Clone 1955598 (SEQ ID NO:334), gill 174870 (SEQ ID NO:335), and Clone 1739308 (SEQ ID NO:337).

FIG. 12 is an alignment of Clone 36334 (SEQ ID NO:229) with homologous and/or orthologous amino acid sequences Clone 690176 (SEQ ID NO:230), Annot 1464715 (SEQ ID NO:232), gi|9587211 (SEQ ID NO:234), gi|45260636 (SEQ ID NO:238), gi|86279652 (SEQ ID NO:239), gi|60677685 (SEQ ID NO:241), Clone 339347 (SEQ ID NO:242), gi|70609692 (SEQ ID NO:338), and Clone 1786280 (SEQ ID NO:340).

FIG. 13 is an alignment of Clone 37493 (SEQ ID NO:245) with homologous and/or orthologous amino acid sequences Annot 1494370 (SEQ ID NO:247) and gi|50929439 (SEQ ID NO:248).

FIG. 14 is an alignment of Clone 590462 (SEQ ID NO:80) with homologous and/or orthologous amino acid sequences gi|114974_T (SEQ ID NO:415), gi|92881003_T (SEQ ID NO:416), gi|54290938_T (SEQ ID NO:417), gi|16757966_T (SEQ ID NO:418), gi|54401705_T (SEQ ID NO:420), Annot 1437978_T (SEQ ID NO:421), gi|6118076_T (SEQ ID NO:422), gi|32400332_T (SEQ ID NO:423), gi|110623260_T (SEQ ID NO:424), Clone 1777157_T (SEQ ID NO:425), Clone 732610_T (SEQ ID NO:426), Clone 1926430_T (SEQ ID NO:427), gi|6840855_T (SEQ ID NO:428), Clone 327253_T (SEQ ID NO:429), gi|249262_T (SEQ ID NO:430), gi|28628597_T (SEQ ID NO:431), gi|127734_T (SEQ ID NO:433), gi|17226270_T (SEQ ID NO:434), gi|127733_T (SEQ ID NO:437), gi|71361195_T (SEQ ID NO:439), gi|56112345_T (SEQ ID NO:440), and gill 1034734_T (SEQ ID NO:441).

FIG. 15 is an alignment of Clone 590462_FL (SEQ ID NO:414) with homologous and/or orthologous amino acid sequences Annot 1437978 (SEQ ID NO:369), Clone 1777157 (SEQ ID NO:373), Clone 1926430 (SEQ ID NO:377), Clone 327253 (SEQ ID NO:379), Clone 732610 (SEQ ID NO:381), gi|11034734 (SEQ ID NO:382), gi|110623260 (SEQ ID NO:384), gi|114974 (SEQ ID NO:385), gill 155255 (SEQ ID NO:386), gi|12621052 (SEQ ID NO:387), gi|127733 (SEQ ID NO:388), gi|127734 (SEQ ID NO:389), gi|15778634 (SEQ ID NO:390), gi|17226270 (SEQ ID NO:392), gi|249262 (SEQ ID NO:393), gi|28628597 (SEQ ID NO:394), gi|32400332 (SEQ ID NO:395), gi|54290938 (SEQ ID NO:398), gi|54401705 (SEQ ID NO:399), gi|56112345 (SEQ ID NO:400), gi|56130949 (SEQ ID NO:401), gi|6103585 (SEQ ID NO:403), gi|6118076 (SEQ ID NO:404), gi|62131643 (SEQ ID NO:405), gi|6840855 (SEQ ID NO:406), gi|71361195 (SEQ ID NO:407), gi|74473455 (SEQ ID NO:408), gi|84316715 (SEQ ID NO:409), and gi|92881003 (SEQ ID NO:412).

### DETAILED DESCRIPTION

The invention features methods and materials related to modulating (e.g., increasing or decreasing) oil levels in plants. In some embodiments, the plants may also have modulated levels of protein. The methods can include transforming a plant cell with a nucleic acid encoding an oil-modulating polypeptide, wherein expression of the polypeptide results in a modulated level of oil. Plant cells produced using such methods can be grown to produce plants having an increased or decreased oil content. Seeds from such plants may be used to produce, for example, foodstuffs and animal feed having an increased oil content. Producing oil from seeds having an increased oil content can allow manufacturers to increase oil yields.

### Polypeptides

The term “polypeptide” as used herein refers to a compound of two or more subunit amino acids, amino acid analogs, or other peptidomimetics, regardless of post-translational modification, e.g., phosphorylation or glycosylation. The subunits may be linked by peptide bonds or other bonds such as, for example, ester or ether bonds. The term “amino acid” refers to natural and/or unnatural or synthetic amino acids, including D/L optical isomers. Full-length proteins, analogs, mutants, and fragments thereof are encompassed by this definition.

Polypeptides described herein include oil-modulating polypeptides. Oil-modulating polypeptides can be effective to modulate oil levels when expressed in a plant or plant cell. Modulation of the level of oil can be either an increase or a decrease in the level of oil relative to the corresponding level in a control plant.

An oil-modulating polypeptide can contain an AP2 domain characteristic of polypeptides belonging to the AP2/EREBP family of plant transcription factor polypeptides. AP2 (APETALA2) and EREBPs (ethylene-responsive element binding proteins) are prototypic members of a family of transcription factors unique to plants, whose distinguishing characteristic is that they contain the so-called AP2 DNA binding domain. AP2/EREBP genes form a large multigene family encoding polypeptides that play a variety of roles throughout the plant life cycle: from being key regulators of several developmental processes, such as floral organ identity determination and control of leaf epidermal cell identity, to forming part of the mechanisms used by plants to respond to various types of biotic and environmental stress.

SEQ ID NO:82 sets forth the amino acid sequence of a Glycine max clone, identified herein as Ceres CLONE ID no. 625035 (SEQ ID NO:81), that is predicted to encode an AP2/EREBP transcription factor polypeptide. An oil-modulating polypeptide can comprise the amino acid sequence set forth in SEQ ID NO:82. Alternatively, an oil-modulating polypeptide can be a homolog, ortholog, or variant of the polypeptide having the amino acid sequence set forth in SEQ ID NO:82. For example, an oil-modulating polypeptide can have an amino acid sequence with at least 50% sequence identity, e.g., 51%, 55%, 60%, 65%, 70%, 75%, 80%, 85%, 90%, 95%, 97%, 98%, or 99% sequence identity, to the amino acid sequence set forth in SEQ ID NO:82.

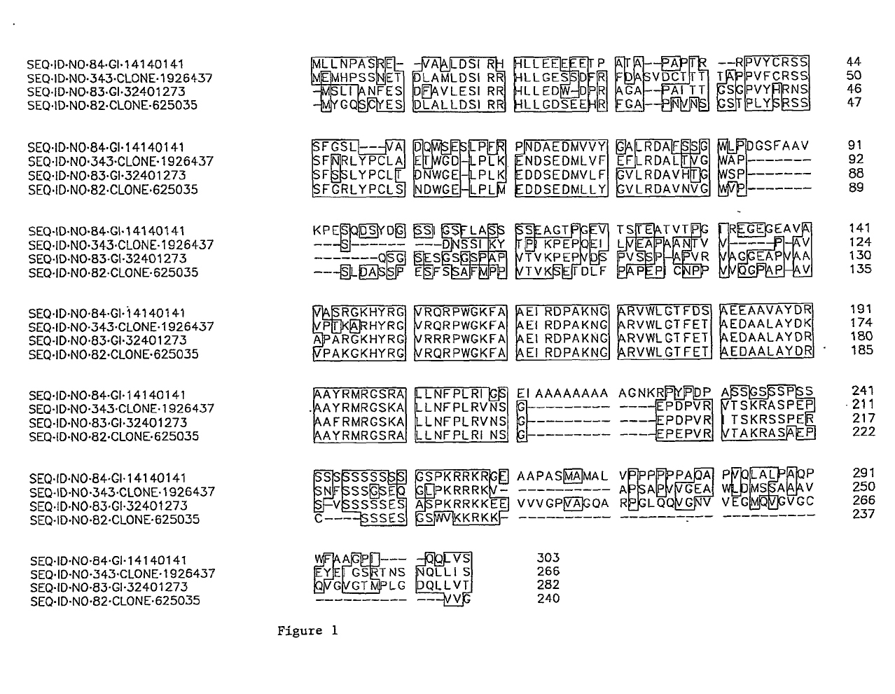

Amino acid sequences of homologs and/or orthologs of the polypeptide having the amino acid sequence set forth in SEQ ID NO:82 are provided in FIG. 1. The alignment in FIG. 1 provides the amino acid sequences of Clone 625035 (SEQ ID NO:82), gi|32401273 (SEQ ID NO:83), gi|14140141 (SEQ ID NO:84), and Clone 1926437 (SEQ ID NO:343). Other homologs and/or orthologs include gi|50911399 (SEQ ID NO:85) and gi|7528276 (SEQ ID NO:341).

In some cases, an oil-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:83, SEQ ID NO:84, SEQ ID NO:85, SEQ ID NO:341, or SEQ ID NO:343.

An oil-modulating polypeptide can contain a leucine-rich repeat, such as LRR_{—}1. Leucine-rich repeats (LRR) consist of 2-45 motifs of 20-30 amino acids that generally fold into an arc or horseshoe shape and are often flanked by cysteine rich domains. Each LRR is composed of a beta-alpha unit. LRRs appear to provide a structural framework for the formation of protein-protein interactions. Polypeptides containing LRRs include tyrosine kinase receptors, cell-adhesion molecules, virulence factors, and extracellular matrix-binding glycoproteins that are involved in a variety of biological processes, including signal transduction, cell adhesion, DNA repair, recombination, transcription, RNA processing, and disease resistance.

SEQ ID NO:87 sets forth the amino acid sequence of an Arabidopsis clone, identified herein as Ceres CLONE ID no. 5344 (SEQ ID NO:86), that is predicted to encode a polypeptide containing a leucine-rich repeat. An oil-modulating polypeptide can comprise the amino acid sequence set forth in SEQ ID NO: 87. Alternatively, an oil-modulating polypeptide can be a homolog, ortholog, or variant of the polypeptide having the amino acid sequence set forth in SEQ ID NO:87. For example, an oil-modulating polypeptide can have an amino acid sequence with at least 45% sequence identity, e.g., 45%, 50%, 55%, 60%, 65%, 70%, 75%, 80%, 85%, 90%, 95%, 97%, 98%, or 99% sequence identity, to the amino acid sequence set forth in SEQ ID NO:87.

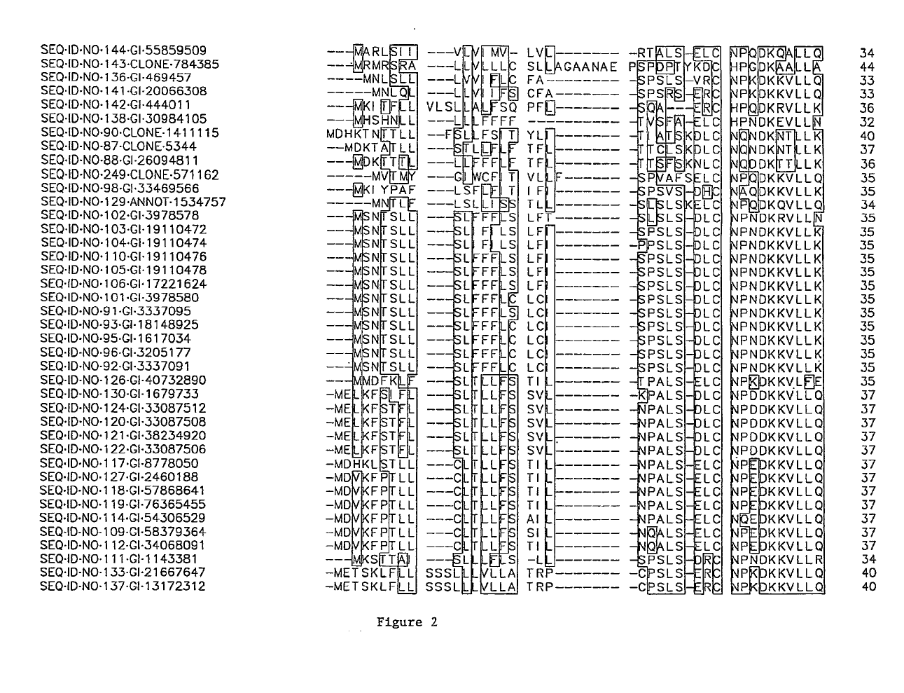

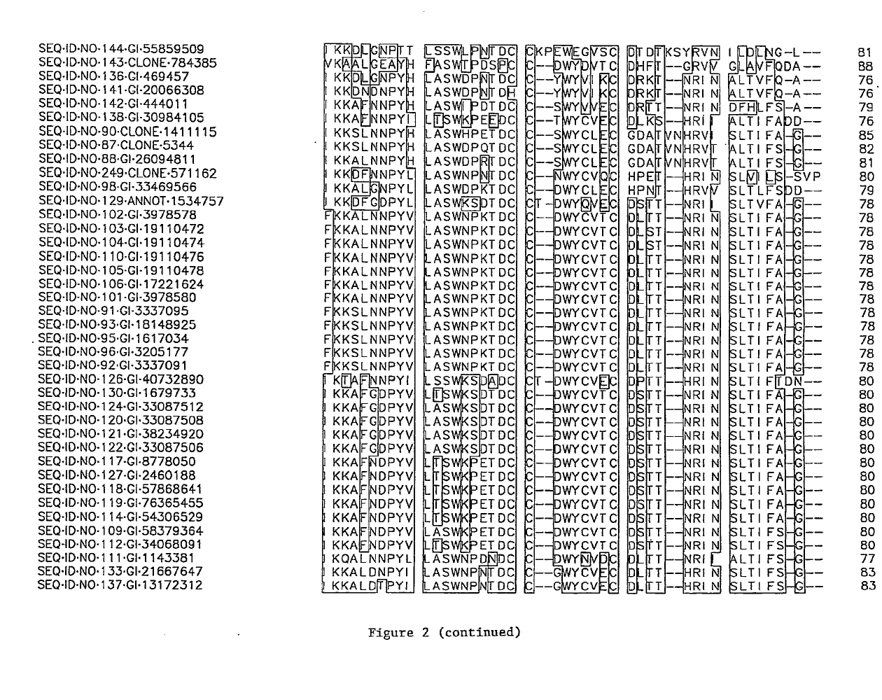

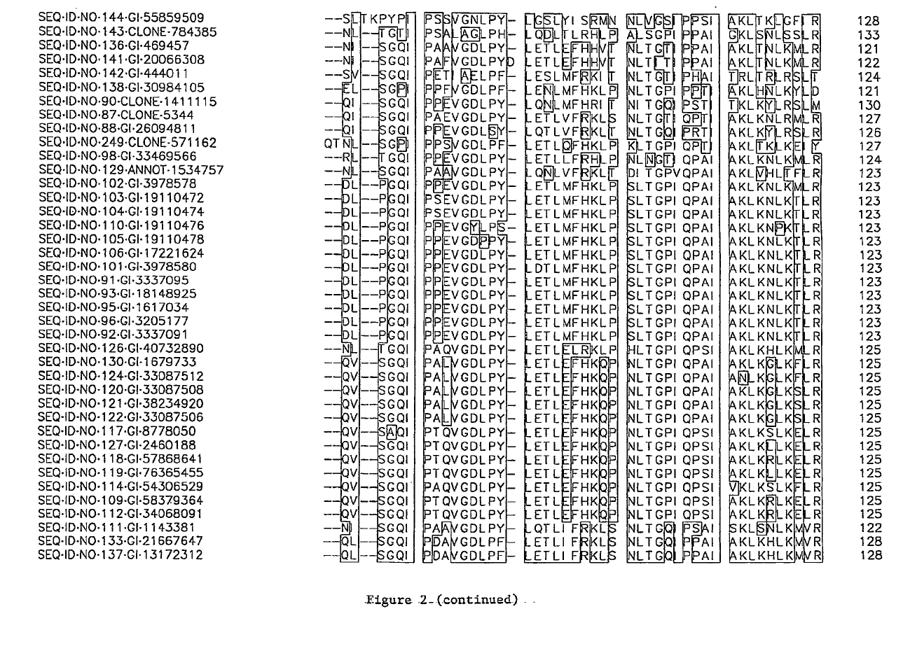

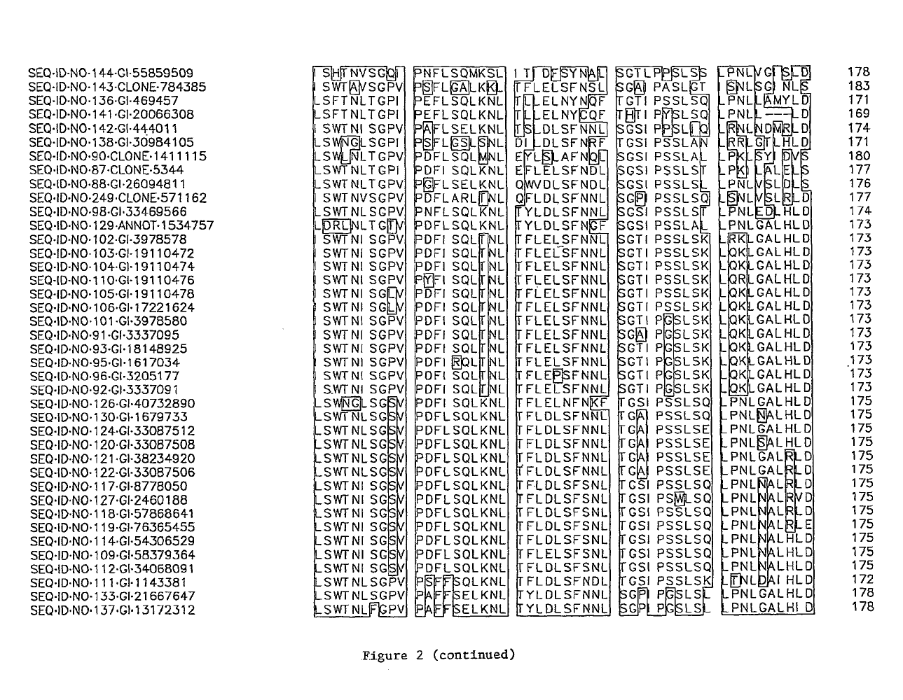

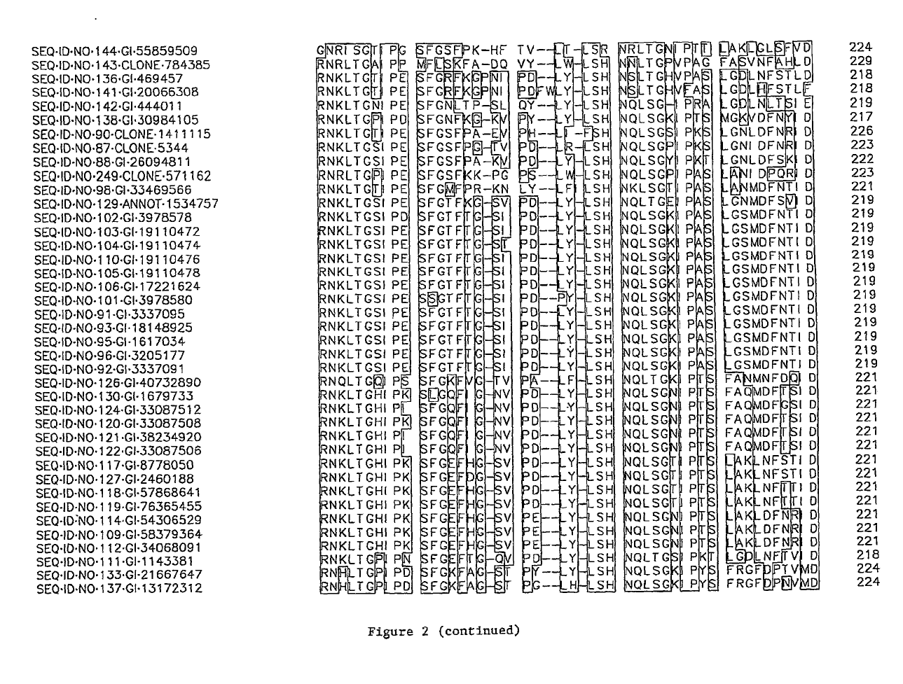

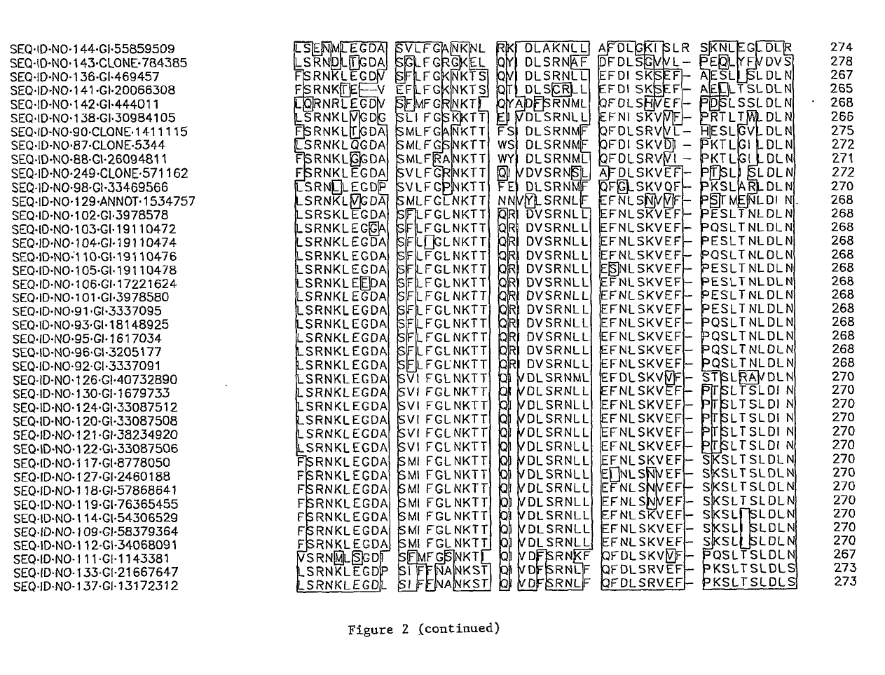

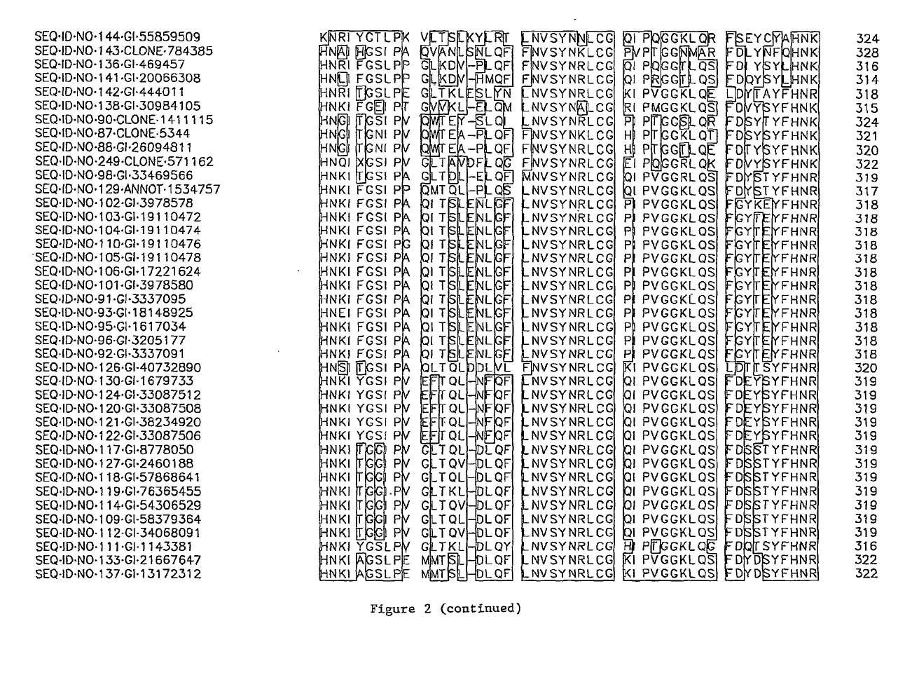

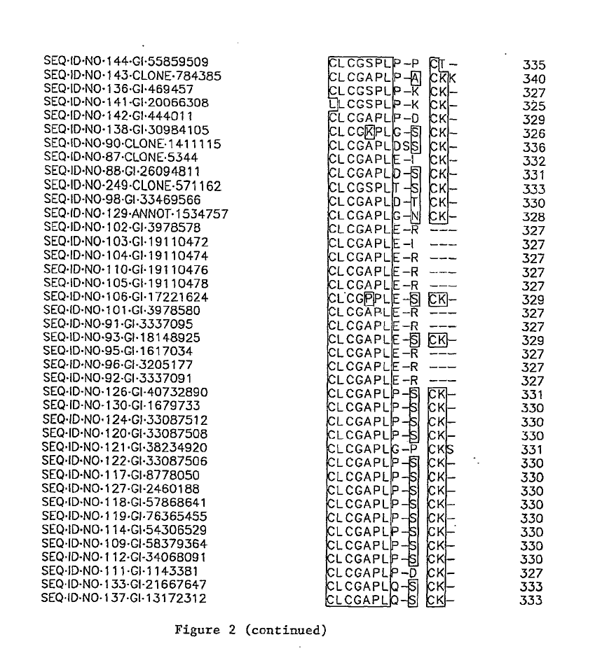

Amino acid sequences of homologs and/or orthologs of the polypeptide having the amino acid sequence set forth in SEQ ID NO:87 are provided in FIG. 2. The alignment in FIG. 2 provides the amino acid sequences of Clone 5344 (SEQ ID NO:87), gi|26094811 (SEQ ID NO:88), Clone 1411115 (SEQ ID NO:90), gi|3337095 (SEQ ID NO:91), gi|3337091 (SEQ ID NO:92), gill 8148925 (SEQ ID NO:93), gi|1617034 (SEQ ID NO:95), gi|3205177 (SEQ ID NO:96), gi|33469566 (SEQ ID NO:98), gi|3978580 (SEQ ID NO:101), gi|3978578 (SEQ ID NO:102), gi|19110472 (SEQ ID NO:103), gi|19110474 (SEQ ID NO:104), gi|19110478 (SEQ ID NO:105), gi|17221624 (SEQ ID NO:106), gi|58379364 (SEQ ID NO:109), gi|19110476 (SEQ ID NO:110), gi 1143381 (SEQ ID NO:111), gi|34068091 (SEQ ID NO:112), gi|54306529 (SEQ ID NO:114), gi|8778050 (SEQ ID NO:117), gi|57868641 (SEQ ID NO:118), gi|76365455 (SEQ ID NO:119), gi|33087508 (SEQ ID NO:120), gi|38234920 (SEQ ID NO:121), gi|33087506 (SEQ ID NO:122), gi|33087512 (SEQ ID NO:124), gi|40732890 (SEQ ID NO:126), gi|2460188 (SEQ ID NO:127), Annot 1534757 (SEQ ID NO:129), gi|1679733 (SEQ ID NO:130), gi|21667647 (SEQ ID NO:133), gi|469457 (SEQ ID NO:136), gi|13172312 (SEQ ID NO:137), gi|30984105 (SEQ ID NO:138), gi|20066308 (SEQ ID NO:141), gi|444011 (SEQ ID NO:142), Clone 784385 (SEQ ID NO:143), gi|55859509 (SEQ ID NO:144), and Clone 571162 (SEQ ID NO:249). Other homologs and/or orthologs include Ceres CLONE ID no. 1301219 (SEQ ID NO:89), Public GI no. 3242641 (SEQ ID NO:94), Public GI no. 18148376 (SEQ ID NO:97), Public GI no. 3337093 (SEQ ID NO:99), Public GI no. 18148923 (SEQ ID NO:100), Public GI no. 3192102 (SEQ ID NO:107), Public GI no. 17221626 (SEQ ID NO:108), Public GI no. 58379362 (SEQ ID NO:113), Public GI no. 58379372 (SEQ ID NO:115), Public GI no. 6651282 (SEQ ID NO:116), Public GI no. 63099931 (SEQ ID NO:123), Public GI no. 33087510 (SEQ ID NO:125), Ceres ANNOT ID no. 1481274 (SEQ ID NO:132), Ceres ANNOT ID no. 1528311 (SEQ ID NO:135), Ceres ANNOT ID no. 1474878 (SEQ ID NO:140), Public GI no. 50871748 (SEQ ID NO:145), Public GI no. 55859507 (SEQ ID NO:146), Public GI ID no. 33469564 (SEQ ID NO:344) and Ceres CLONE ID no. 1820701 (SEQ ID NO:346).

In some cases, an oil-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ED NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ BD NO:125, SEQ ID NO:126, SEQ ID NO:127, SEQ ID NO:129, SEQ ID NO:130, SEQ ID NO:132, SEQ ID NO:133, SEQ ID NO:135, SEQ ID NO:136, SEQ ID NO:137, SEQ ID NO:138, SEQ ID NO:140, SEQ ID NO:141, SEQ ID NO:142, SEQ ID NO:143, SEQ ID NO:144, SEQ ID NO:145, SEQ ID NO:146, SEQ ID NO:249, SEQ ID NO:344, or SEQ ID NO:346.

An oil-modulating polypeptide can contain an ankyrin repeat. The ankyrin repeat is one of the most common protein-protein interaction motifs in nature. Ankyrin repeats are tandemly repeated modules of about 33 amino acids. The repeat has been found in polypeptides of diverse function such as transcriptional initiators, cell-cycle regulators, cytoskeletal, ion transporters and signal transducers. Each repeat folds into a helix-loop-helix structure with a beta-hairpin/loop region projecting out from the helices at a 90 degree angle. The repeats stack together to form an L-shaped structure.

SEQ ID NO:148 sets forth the amino acid sequence of an Arabidopsis clone, identified herein as Ceres CDNA ID no. 23649975 (SEQ ID NO:147), that is predicted to encode a polypeptide containing an ankyrin repeat. An oil-modulating polypeptide can comprise the amino acid sequence set forth in SEQ ID NO:148. Alternatively, an oil-modulating polypeptide can be a homolog, ortholog, or variant of the polypeptide having the amino acid sequence set forth in SEQ ID NO:148. For example, an oil-modulating polypeptide can have an amino acid sequence with at least 70% sequence identity, e.g., 75%, 80%, 85%, 90%, 95%, 97%, 98%, or 99% sequence identity, to the amino acid sequence set forth in SEQ ID NO:148.

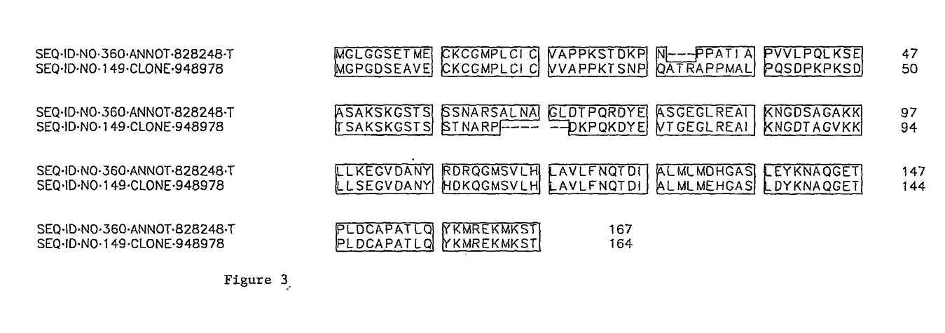

The amino acid sequences of homologs and/or orthologs of the polypeptide having the amino acid sequence set forth in SEQ ID NO:148 are provided in FIG. 3. The alignment in FIG. 3 provides the amino acid sequences of Annot 828248_T (SEQ ID NO:360) and Clone 948978 (SEQ ID NO:149).

In some cases, an oil-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:149 or SEQ ID NO:360.

An oil-modulating polypeptide can contain a glycosyltransferase family 28 C-terminal domain, Glyco_tran_{—}28_C, characteristic of a glycosyltransferase polypeptide belonging to the glycosyltransferase family 28. Glycosyltransferase polypeptides are enzymes that catalyze the transfer of sugar moieties from activated donor molecules to specific acceptor molecules, forming glycosidic bonds. Glycosyltransferase family 28 comprises enzymes with a number of known activities: 1,2-diacylglycerol 3-beta-galactosyltransferase, 1,2-diacylglycerol 3-beta-glucosyltransferase, and beta-N-acetylglucosamine transferase. Results of structural analyses suggest that the C-terminal domain contains the UDP-GlcNAc binding site. The 3-D structures of glycosyltransferase polypeptides are better conserved than the sequences of glycosyltransferase polypeptides.

SEQ ID NO:162 sets forth the amino acid sequence of an Arabidopsis clone, identified herein as Ceres CDNA ID no. 12706677 (SEQ ID NO:161), that is predicted to encode a glycosyltransferase polypeptide. An oil-modulating polypeptide can comprise the amino acid sequence set forth in SEQ ID NO:162. Alternatively, an oil-modulating polypeptide can be a homolog, ortholog, or variant of the polypeptide having the amino acid sequence set forth in SEQ ID NO:162. For example, an oil-modulating polypeptide can have an amino acid sequence with at least 60% sequence identity, e.g., 61%, 65%, 70%, 75%, 80%, 85%, 90%, 95%, 97%, 98%, or 99% sequence identity, to the amino acid sequence set forth in SEQ ID NO:162.

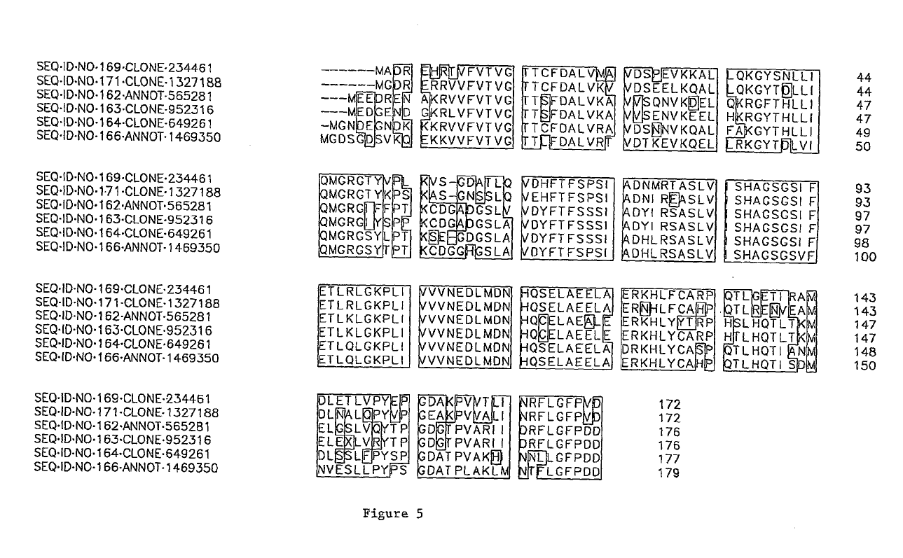

Amino acid sequences of homologs and/or orthologs of the polypeptide having the amino acid sequence set forth in SEQ ID NO:162 are provided in FIG. 5. The alignment in FIG. 5 provides the amino acid sequences of Annot 565281 (SEQ ID NO:162), Clone 952316 (SEQ ID NO:163), Clone 649261 (SEQ ID NO:164), Annot 1469350 (SEQ ID NO:166), Clone 234461 (SEQ ID NO:169), and Clone 1327188 (SEQ ID NO:171). Other homologs and/or orthologs include Ceres ANNOT ID no. 1488942 (SEQ ID NO:168), Ceres CLONE ID no. 217678 (SEQ ID NO:170), Ceres CLONE ID no. 1831965 (SEQ ID NO:355), Ceres CLONE ID no. 1770078 (SEQ ID NO:357), and Ceres CLONE ID no. 2008759 (SEQ ID NO:359).

In some cases, an oil-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:163, SEQ ID NO:164, SEQ ID NO:166, SEQ ID NO:168, SEQ ID NO:169, SEQ ID NO:170, SEQ ID NO:171, SEQ ID NO:355, SEQ ID NO:357, or SEQ ID NO:359.

An oil-modulating polypeptide can contain an Acetyltransf_{—}1 domain. The Acetyltransf_{—}1 domain is characteristic of polypeptides belonging to the acetyltransferase (GNAT) family. The GNAT family includes GcnS-related acetyltransferases, which catalyze the transfer of an acetyl group from acetyl-CoA to the lysine E-amino groups on the N-terminal tails of histones. Many GNATs share several functional domains, including an N-terminal region of variable length, an acetyltransferase domain encompassing conserved sequence motifs, a region that interacts with the coactivator Ada2, and a C-terminal bromodomain that is believed to interact with acetyl-lysine residues. Members of the GNAT family are important for the regulation of cell growth and development. The importance of GNATs is probably related to their role in transcription and DNA repair. SEQ ID NO:185 sets forth the amino acid sequence of an Arabidopsis clone, identified herein as Ceres ANNOT ID no. 549258 (SEQ ID NO:362), that is predicted to encode an acetyltransferase polypeptide. An oil-modulating polypeptide can comprise the amino acid sequence set forth in SEQ ID NO:185. Alternatively, an oil-modulating polypeptide can be a homolog, ortholog, or variant of the polypeptide having the amino acid sequence set forth in SEQ ID NO:185. For example, an oil-modulating polypeptide can have an amino acid sequence with at least 55% sequence identity, e.g., 55%, 60%, 65%, 70%, 75%, 80%, 85%, 90%, 95%, 97%, 98%, or 99% sequence identity, to the amino acid sequence set forth in SEQ ID NO:185.

Amino acid sequences of homologs and/or orthologs of the polypeptide having the amino acid sequence set forth in SEQ ID NO:185 are provided in FIG. 7.

The alignment in FIG. 7 provides the amino acid sequences of Annot 549258 (SEQ ID NO:185), Clone 945519 (SEQ ID NO:186), and gi|50935585 (SEQ ID NO:187). Other homologs and/or orthologs include Public GI no. 51963354 (SEQ ID NO:188).

In some cases, an oil-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:186, SEQ ID NO:187, SEQ ID NO:188.

An oil-modulating polypeptide can contain a DnaJ domain associated with chaperone polypeptides involved in protein folding. SEQ ID NO:190 sets forth the amino acid sequence of an Arabidopsis clone, identified herein as Ceres ANNOT ID no. 564261 (SEQ ID NO:364), that is predicted to encode a polypeptide containing a DnaJ domain. An oil-modulating polypeptide can comprise the amino acid sequence set forth in SEQ ID NO:190. Alternatively, an oil-modulating polypeptide can be a homolog, ortholog, or variant of the polypeptide having the amino acid sequence set forth in SEQ ID NO:190. For example, an oil-modulating polypeptide can have an amino acid sequence with at least 55% sequence identity, e.g., 55%, 60%, 65%, 70%, 75%, 80%, 85%, 90%, 95%, 97%, 98%, or 99% sequence identity, to the amino acid sequence set forth in SEQ ID NO:190.

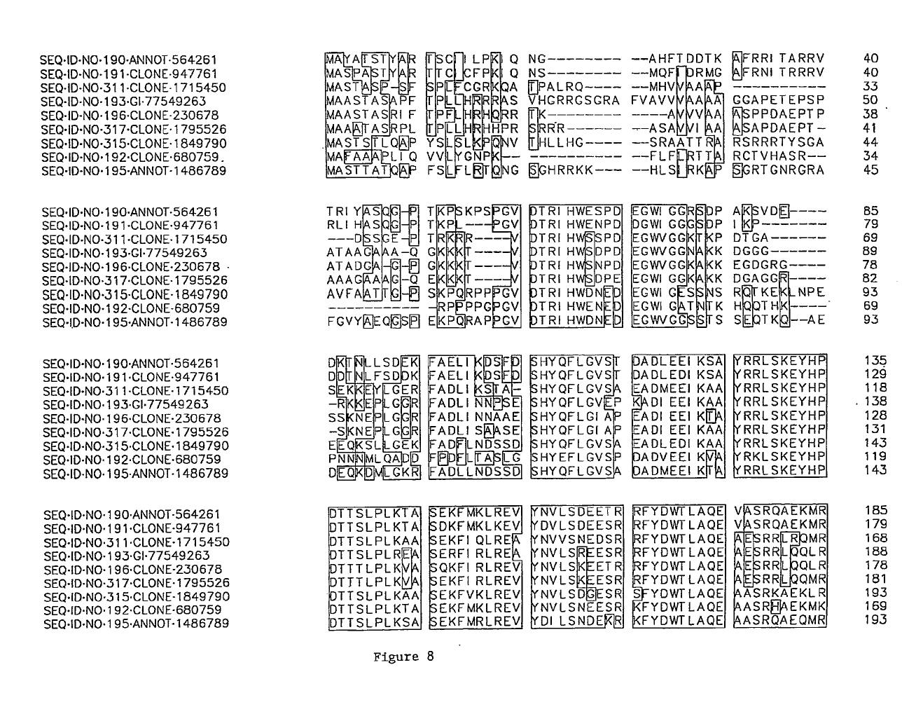

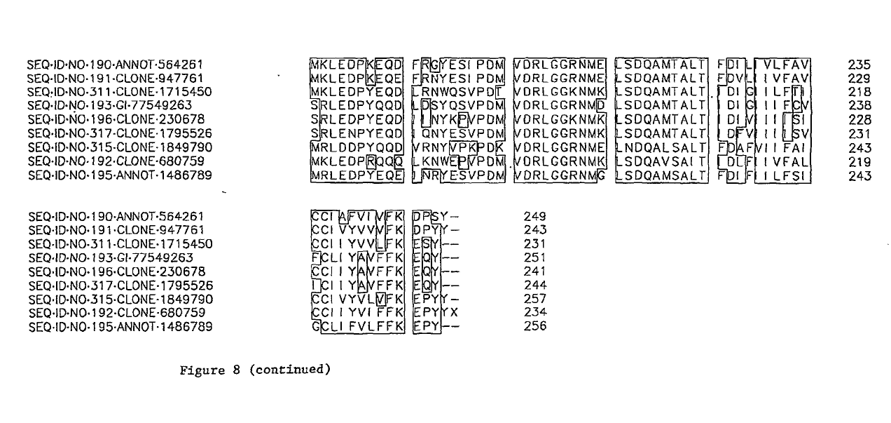

Amino acid sequences of homologs and/or orthologs of the polypeptide having the amino acid sequence set forth in SEQ ID NO:190 are provided in FIG. 8. The alignment in FIG. 8 provides the amino acid sequences of Annot 564261 (SEQ ID NO:190), Clone 947761 (SEQ ID NO:191), Clone 680759 (SEQ ID NO:192), gi|77549263 (SEQ ID NO:193), Annot 1486789 (SEQ ID NO:195), Clone 230678 (SEQ ID NO:196), Clone 1715450 (SEQ ID NO:311), Clone 1849790 (SEQ ID NO:315), and Clone 1795526 (SEQ ID NO:317).

In some cases, an oil-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:191, SEQ ID NO:192, SEQ ID NO:193, SEQ ID NO:195, SEQ ID NO:196, SEQ ID NO:311, SEQ ID NO:315, or SEQ ID NO:317.

An oil-modulating polypeptide can have a Rho_N domain found in the N-terminus of the Rho termination factor. The Rho termination factor disengages newly transcribed RNA from its DNA template at certain, specific transcripts. It is thought that two copies of Rho bind to RNA and that Rho functions as a hexamer of protomers.

SEQ ID NO:198 sets forth the amino acid sequence of an Arabidopsis clone, identified herein as Ceres ANNOT ID no. 565548 (SEQ ID NO:365), that is predicted to encode a polypeptide containing a Rho_N domain. An oil-modulating polypeptide can comprise the amino acid sequence set forth in SEQ ID NO:198. Alternatively, an oil-modulating polypeptide can be a homolog, ortholog, or variant of the polypeptide having the amino acid sequence set forth in SEQ ID NO:198. For example, an oil-modulating polypeptide can have an amino acid sequence with at least 60% sequence identity, e.g., 60%, 65%, 70%, 75%, 80%, 85%, 90%, 95%, 97%, 98%, or 99% sequence identity, to the amino acid sequence set forth in SEQ ID NO:198.

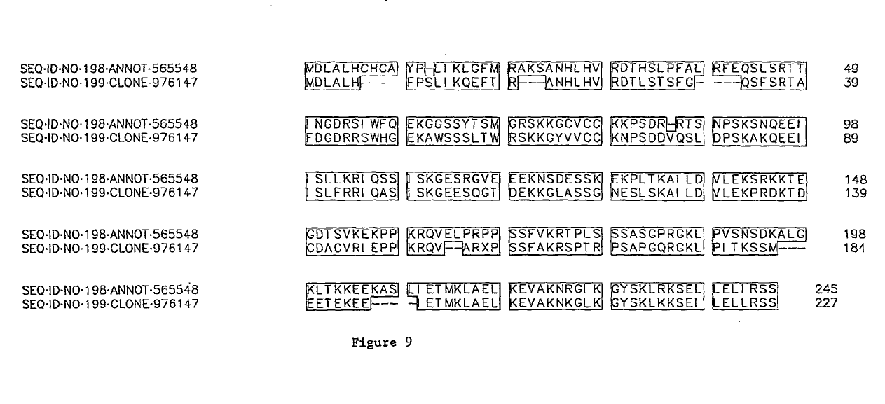

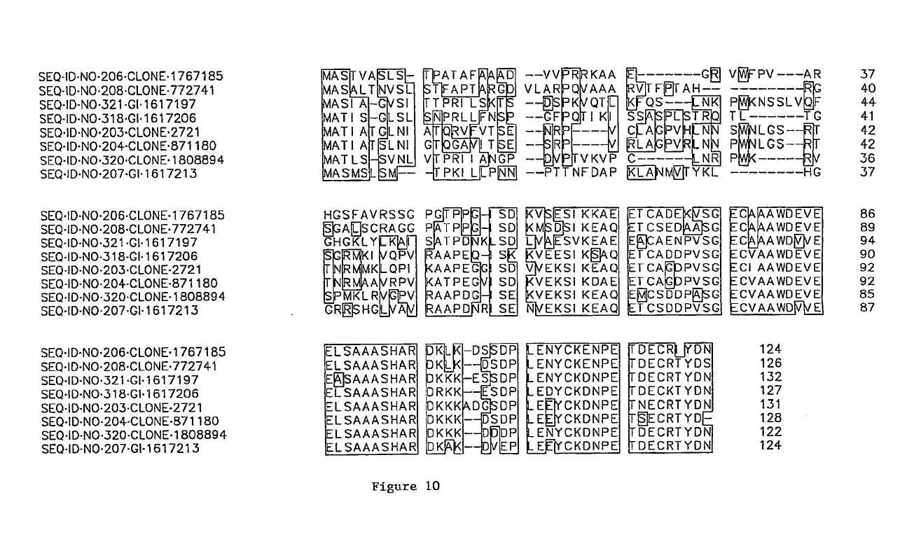

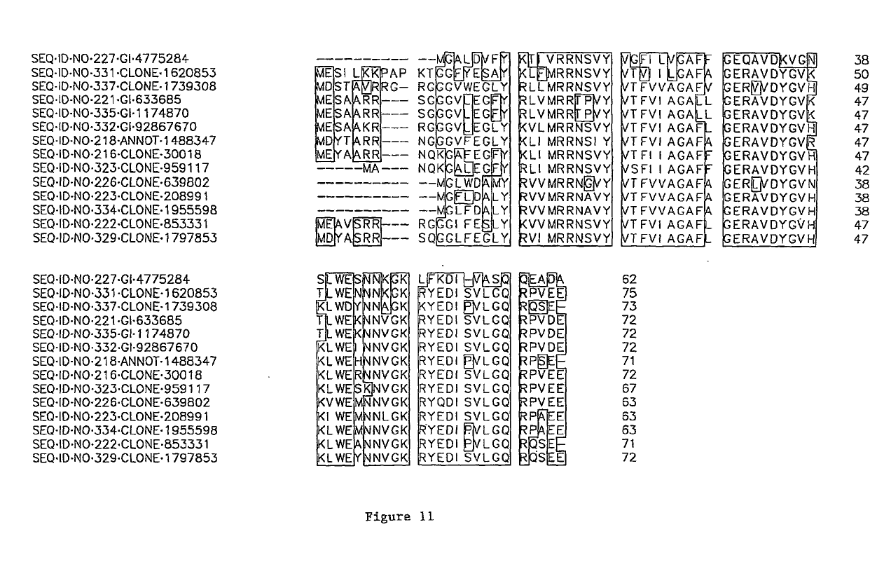

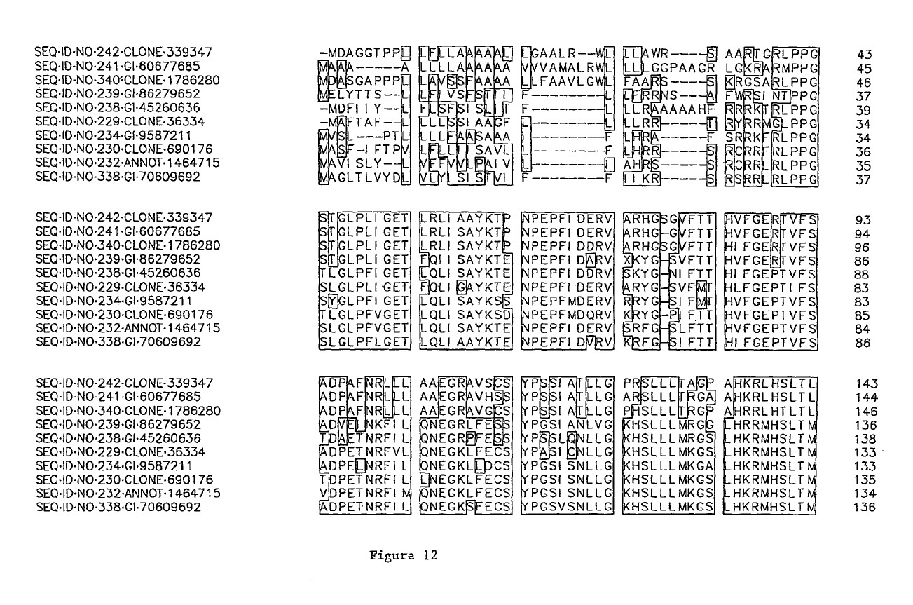

The amino acid sequence of a homolog and/or ortholog of the polypeptide having the amino acid sequence set forth in SEQ ID NO:198 is provided in FIG. 9. The alignment in FIG. 9 provides the amino acid sequences of Annot 565548 (SEQ ID NO:198) and Clone 976147 (SEQ ID NO:199).

In some cases, an oil-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:199.

An oil-modulating polypeptide can have an Exo_endo_phos domain characteristic of polypeptides belonging to the endonuclease/exonuclease/phosphatase family of polypeptides. This large family of polypeptides includes magnesium dependent endonucleases and phosphatases involved in intracellular signaling. For example, the endonuclease/exonuclease/phosphatase family includes AP endonuclease proteins, DNase I proteins, and Synaptojanin, an inositol-1,4,5-trisphosphate phosphatase.

SEQ ID NO:201 sets forth the amino acid sequence of an Arabidopsis clone, identified herein as Ceres ANNOT ID no. 841273 (SEQ ID NO:363), that is predicted to encode a polypeptide containing an Exo_endo_phos domain. An oil-modulating polypeptide can comprise the amino acid sequence set forth in SEQ ID NO:201. Alternatively, an oil-modulating polypeptide can be a homolog, ortholog, or variant of the polypeptide having the amino acid sequence set forth in SEQ ID NO:201. For example, an oil-modulating polypeptide can have an amino acid sequence with at least 40% sequence identity, e.g., 40%, 45%, 50%, 55%, 60%, 65%, 70%, 75%, 80%, 85%, 90%, 95%, 97%, 98%, or 99% sequence identity, to the amino acid sequence set forth in SEQ ID NO:201.

An oil-modulating polypeptide can contain a UCR_UQCRX_QCR9 domain characteristic of a ubiquinol-cytochrome C reductase, UQCRX/QCR9 like polypeptide. The UQCRX/QCR9 polypeptide is part of the mitochondrial respiratory chain. SEQ ID NO:216 sets forth the amino acid sequence of an Arabidopsis clone, identified herein as Ceres CLONE ID no. 30018 (SEQ ID NO:215), that is predicted to encode a polypeptide containing a UCR_UQCRX_QCR9 domain. An oil-modulating polypeptide can comprise the amino acid sequence set forth in SEQ ID NO:216. Alternatively, an oil-modulating polypeptide can be a homolog, ortholog, or variant of the polypeptide having the amino acid sequence set forth in SEQ ID NO:216. For example, an oil-modulating polypeptide can have an amino acid sequence with at least 40% sequence identity, e.g., 41%, 45%, 50%, 55%, 60%, 65%, 70%, 75%, 80%, 85%, 90%, 95%, 97%, 98%, or 99% sequence identity, to the amino acid sequence set forth in SEQ ID NO:216.

Amino acid sequences of homologs and/or orthologs of the polypeptide having the amino acid sequence set forth in SEQ ID NO:216 are provided in FIG. 11. The alignment in FIG. 11 provides the amino acid sequences of Clone 30018 (SEQ ID NO:216), Annot 1488347 (SEQ ID NO:218), gi|633685 (SEQ ID NO:221), Clone 853331 (SEQ ID NO:222), Clone 208991 (SEQ ID NO:223), Clone 639802 (SEQ ID NO:226), gi|4775284 (SEQ ID NO:227), Clone 959117 (SEQ ID NO:323), Clone 1797853 (SEQ ID NO:329), Clone 1620853 (SEQ ID NO:331), gi|92867670 (SEQ ID NO:332), Clone 1955598 (SEQ ID NO:334), gi|1174870 (SEQ ID NO:335), and Clone 1739308 (SEQ ID NO:337). Other homologs and/or orthologs include Ceres ANNOT ID no. 1513719 (SEQ ID NO:220), Ceres CLONE ID no. 336493 (SEQ ID NO:224), Ceres CLONE ID no. 1064967 (SEQ ID NO:225), Ceres CLONE ID no. 1090391 (SEQ ID NO:325), and Ceres CLONE ID no. 1270157 (SEQ ID NO:327).

In some cases, an oil-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:218, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:323, SEQ ID NO:325, SEQ ID NO:327, SEQ ID NO:329, SEQ ID NO:331, SEQ ID NO:332, SEQ ID NO:334, SEQ ID NO:335, or SEQ ID NO:337.

An oil-modulating polypeptide can contain a p450 domain characteristic of a cytochrome P450 polypeptide. The cytochrome P450 enzymes constitute a superfamily of haem-thiolate proteins. P450 enzymes usually act as terminal oxidases in multicomponent electron transfer chains, called P450-containing monooxygenase systems, and are involved in metabolism of a plethora of both exogenous and endogenous compounds. The conserved core is composed of a coil referred to as the “meander,” a four-helix bundle, helices J and K, and two sets of beta-sheets. These regions constitute the haem-binding loop (with an absolutely conserved cysteine that serves as the 5th ligand for the haem iron), the proton-transfer groove, and the absolutely conserved EXXR motif in helix K.

SEQ ID NO:229 sets forth the amino acid sequence of an Arabidopsis clone, identified herein as Ceres CLONE ID no. 36334 (SEQ ID NO:228), that is predicted to encode a cytochrome P450 polypeptide. An oil-modulating polypeptide can comprise the amino acid sequence set forth in SEQ ID NO:229. Alternatively, an oil-modulating polypeptide can be a homolog, ortholog, or variant of the polypeptide having the amino acid sequence set forth in SEQ ID NO:229. For example, an oil-modulating polypeptide can have an amino acid sequence with at least 55% sequence identity, e.g., 60%, 65%, 70%, 75%, 80%, 85%, 90%, 95%, 97%, 98%, or 99% sequence identity, to the amino acid sequence set forth in SEQ ID NO:229.

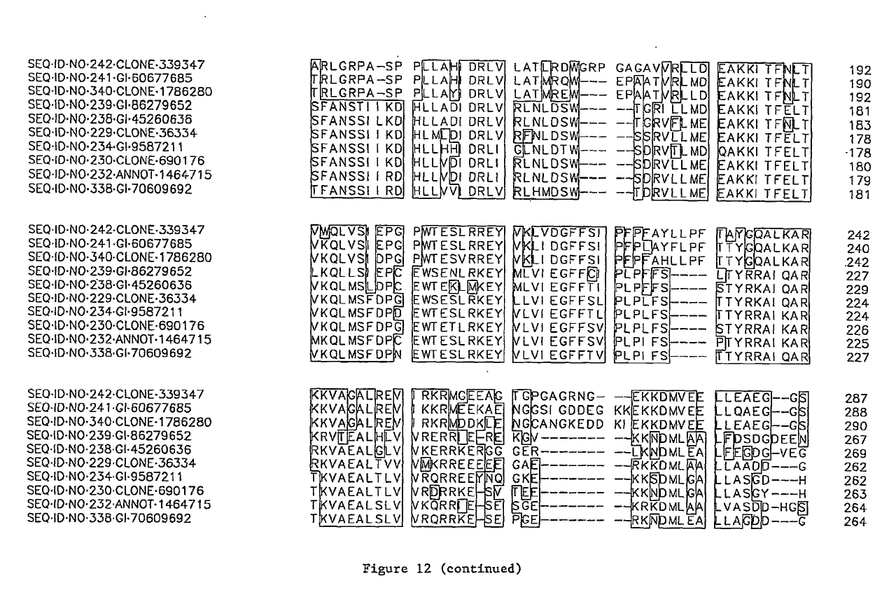

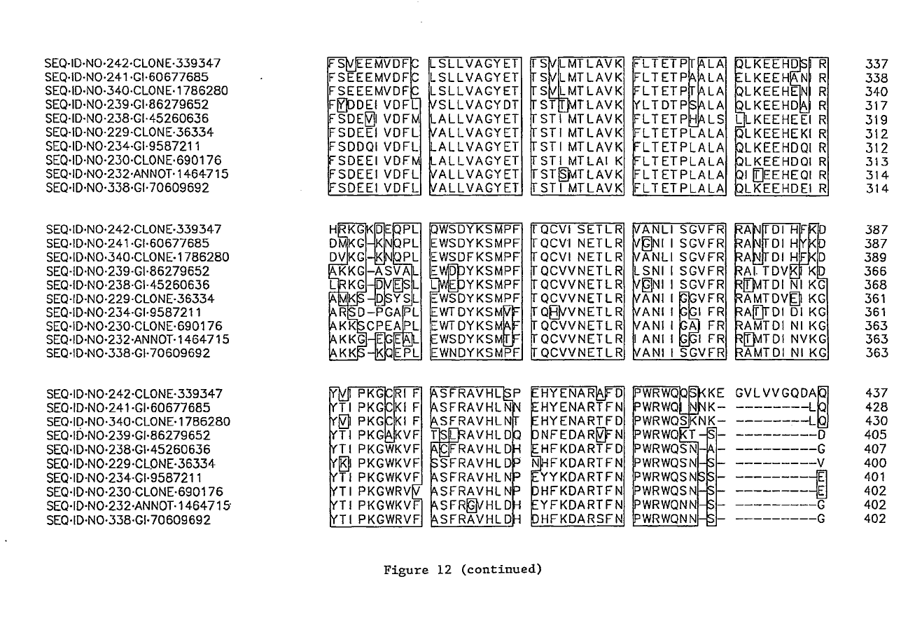

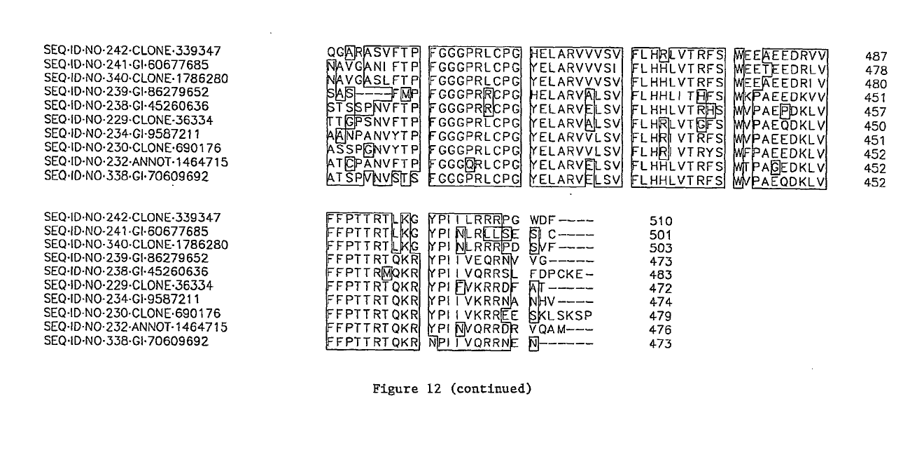

Amino acid sequences of homologs and/or orthologs of the polypeptide having the amino acid sequence set forth in SEQ ID NO:229 are provided in FIG. 12. The alignment in FIG. 12 provides the amino acid sequences of Clone 36334 (SEQ ID NO:229), Clone 690176 (SEQ ID NO:230), Annot 1464715 (SEQ ID NO:232), gi|9587211 (SEQ ID NO:234), gi|45260636 (SEQ ID NO:238), gi|86279652 (SEQ ID NO:239), gi|60677685 (SEQ ID NO:241), Clone 339347 (SEQ ID NO:242), gi|70609692 (SEQ ID NO:338), and Clone 1786280 (SEQ ID NO:340). Other homologs and/or orthologs include Ceres CLONE ID no. 574698 (SEQ ID NO:233), Ceres CLONE ID no. 718939 (SEQ ID NO:235), Ceres ANNOT ID no. 1511511 (SEQ ID NO:237), Public GI no. 71834072 (SEQ ID NO:240), and Public GI no. 77548615 (SEQ ID NO:243).

In some cases, an oil-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:230, SEQ ID NO:232, SEQ ID NO:233, SEQ ID NO:234, SEQ ID NO:235, SEQ ID NO:237, SEQ ID NO:238, SEQ ID NO:239, SEQ ID NO:240, SEQ ID NO:241, SEQ ID NO:242, SEQ ID NO:243, SEQ ID NO:338, or SEQ ID NO:340.

An oil-modulating polypeptide can contain a Methyltransferase_{—}7 domain characteristic of a SAM dependent carboxyl methyltransferase polypeptide. The SAM dependent carboxyl methyltransferase family of plant methyltransferase polypeptides contains enzymes that act on a variety of substrates including salicylic acid, jasmonic acid and 7-methylxanthine.

SEQ ID NO:245 sets forth the amino acid sequence of an Arabidopsis clone, identified herein as Ceres CLONE ID no. 37493 (SEQ ID NO:244), that is predicted to encode a methyltransferase polypeptide. An oil-modulating polypeptide can comprise the amino acid sequence set forth in SEQ ID NO:245. Alternatively, an oil-modulating polypeptide can be a homolog, ortholog, or variant of the polypeptide having the amino acid sequence set forth in SEQ ID NO:245. For example, an oil-modulating polypeptide can have an amino acid sequence with at least 60% sequence identity, e.g., 60%, 65%, 70%, 75%, 80%, 85%, 90%, 95%, 97%, 98%, or 99% sequence identity, to the amino acid sequence set forth in SEQ ID NO:245.

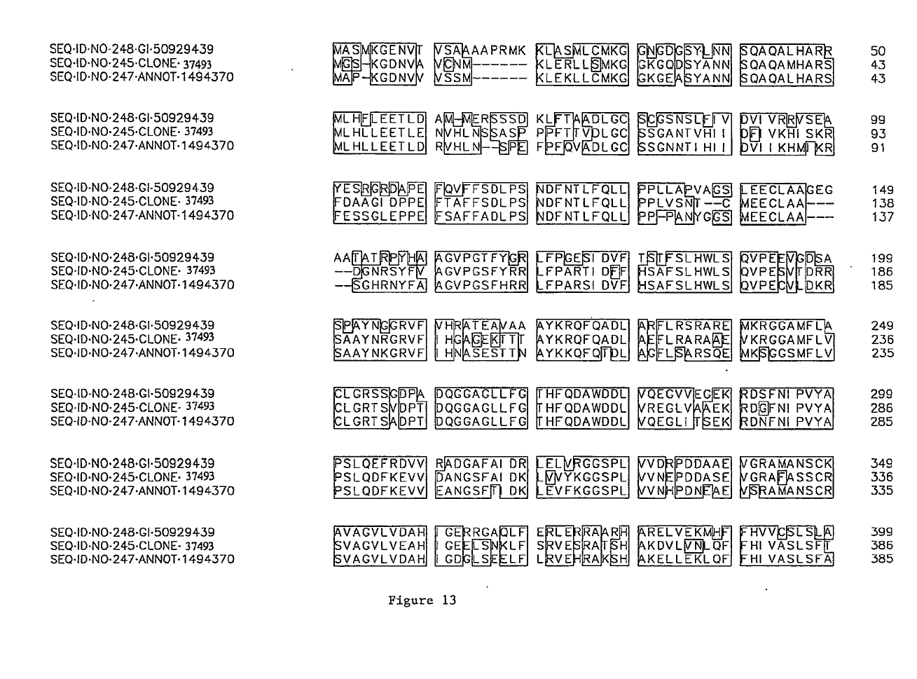

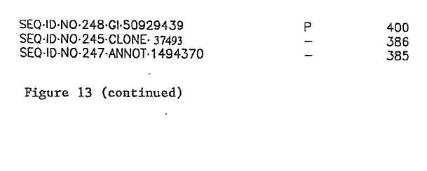

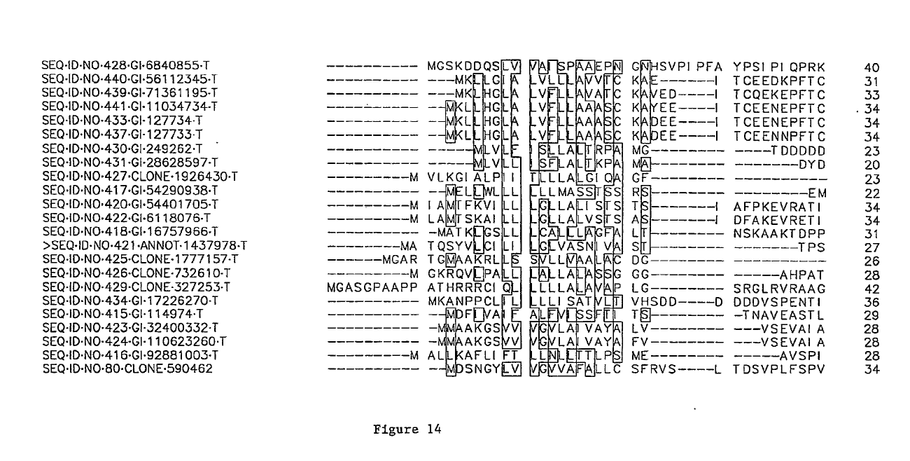

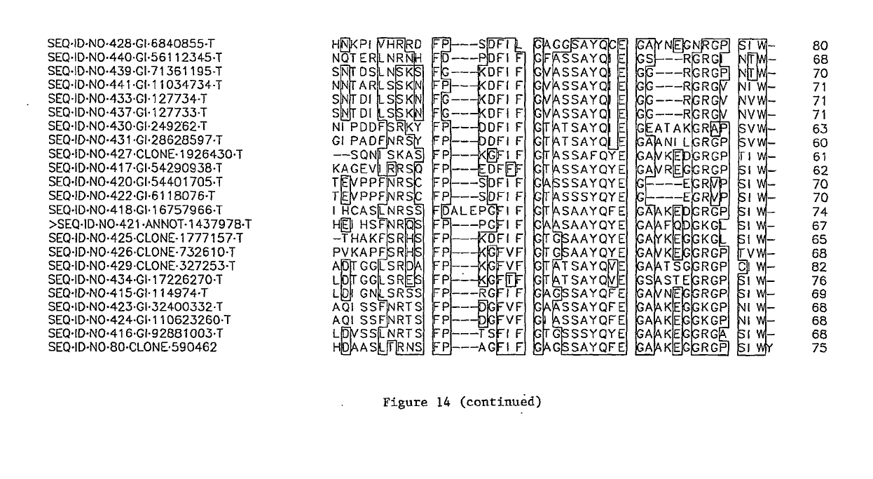

Amino acid sequences of homologs and/or orthologs of the polypeptide having the amino acid sequence set forth in SEQ ID NO:245 are provided in FIG. 13. The alignment in FIG. 13 provides the amino acid sequences of Clone 37493 (SEQ ID NO:245), Annot 1494370 (SEQ ID NO:247), and gi|50929439 (SEQ ID NO:248).

In some cases, an oil-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:247 or SEQ ID NO:248.

An oil-modulating polypeptide can contain a CP12 domain. The CP12 domain contains three conserved cysteines and a histidine, which suggests that the CP12 domain may be a zinc binding domain. SEQ ID NO:203 sets forth the amino acid sequence of an Arabidopsis clone, identified herein as Ceres CLONE ID no. 2721 (SEQ ID NO:202), that is predicted to encode a polypeptide containing a CP12 domain. An oil-modulating polypeptide can comprise the amino acid sequence set forth in SEQ ID NO:203. Alternatively, an oil-modulating polypeptide can be a homolog, ortholog, or variant of the polypeptide having the amino acid sequence set forth in SEQ ID NO:203. For example, an oil-modulating polypeptide can have an amino acid sequence with at least 50% sequence identity, e.g., 55%, 60%, 65%, 70%, 75%, 80%, 85%, 90%, 95%, 97%, 98%, or 99% sequence identity, to the amino acid sequence set forth in SEQ ID NO:203.

Amino acid sequences of homologs and/or orthologs of the polypeptide having the amino acid sequence set forth in SEQ ID NO:203 are provided in FIG. 10. The alignment in FIG. 10 provides the amino acid sequences of Clone 2721 (SEQ ID NO:203), Clone 871180 (SEQ ID NO:204), Clone 1767185 (SEQ ID NO:206), gi|617213 (SEQ ID NO:207), Clone 772741 (SEQ ID NO:208), gi|1617206 (SEQ ID NO:318), Clone 1808894 (SEQ ID NO:320), and gi|1617197 (SEQ ID NO:321). Other homologs and/or orthologs include Ceres CLONE ID no. 1115650 (SEQ ID NO:205), Ceres CLONE ID no. 1760834 (SEQ ID NO:209), Ceres CLONE ID no. 1762311 (SEQ ID NO:210), Ceres CLONE ID no. 1080241 (SEQ ID NO:211), Ceres CLONE ID no. 960043 (SEQ ID NO:212), Ceres CLONE ID no. 1782555 (SEQ ID NO:213), and Ceres CLONE ID no. 1036232 (SEQ ID NO:214).

In some cases, an oil-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:318, SEQ ID NO:320, or SEQ ID NO:321.

SEQ ID NO:80, SEQ ID NO:151, SEQ ID NO:367, and SEQ ID NO:175 set forth the amino acid sequences of DNA clones, identified herein as Ceres CLONE ID no. 590462 (SEQ ID NO:79), Ceres CDNA ID no. 12703936 (SEQ ID NO:150), Ceres SEED LINE ME11833 (SEQ ID NO:366), and Ceres ANNOT ID no. 542494 (SEQ ID NO:361), respectively, each of which is predicted to encode a polypeptide that does not have homology to an existing protein family based on Pfam analysis. An oil-modulating polypeptide can comprise the amino acid sequence set forth in SEQ ID NO:80, SEQ ID NO:151, SEQ ID NO:367, or SEQ ID NO:175. Alternatively, an oil-modulating polypeptide can be a homolog, ortholog, or variant of the polypeptide having the amino acid sequence set forth in SEQ ID NO:80, SEQ ID NO:151, SEQ ID NO:367, or SEQ ID NO:175. For example, an oil-modulating polypeptide can have an amino acid sequence with at least 40% sequence identity, e.g., 41%, 45%, 50%, 55%, 60%, 65%, 70%, 75%, 80%, 85%, 90%, 95%, 97%, 98%, or 99% sequence identity, to the amino acid sequence set forth in SEQ ID NO:80, SEQ ID NO:151, SEQ ID NO:367, or SEQ ID NO:175.

For example, the alignment in FIG. 14 provides the amino acid sequences of Clone 590462 (SEQ ID NO:80), gi|114974_T (SEQ ID NO:415), gi|92881003_T (SEQ ID NO:416), gi|54290938_T (SEQ ID NO:417), gi|16757966_T (SEQ ID NO:418), gi|54401705_T (SEQ ID NO:420), Annot 1437978_T (SEQ ID NO:421), gi|6118076_T (SEQ ID NO:422), gi|32400332_T (SEQ ID NO:423), gi|110623260_T (SEQ ID NO:424), Clone 1777157_T (SEQ ID NO:425), Clone 732610_T (SEQ ID NO:426), Clone 1926430_T (SEQ ID NO:427), gi|6840855_T (SEQ ID NO:428), Clone 327253_T (SEQ ID NO:429), gi|249262_T (SEQ ID NO:430), gi|28628597_T (SEQ ID NO:431), gi|127734_T (SEQ ID NO:433), gi|17226270_T (SEQ ID NO:434), gi|127733_T (SEQ ID NO:437), gi|71361195_T (SEQ ID NO:439), gi|56112345_T (SEQ ID NO:440), and gi|11034734_T (SEQ ID NO:441). Other homologs and/or orthologs include Ceres ANNOT ID no. 1490788_T (SEQ ID NO:419), Public GI ID no. 11034736_T (SEQ ID NO:432), Public GI ID no. 62131643_T (SEQ ID NO:435), Public GI ID no. 56130951_T (SEQ ID NO:436), and Public GI ID no. 12621052_T (SEQ ID NO:438).

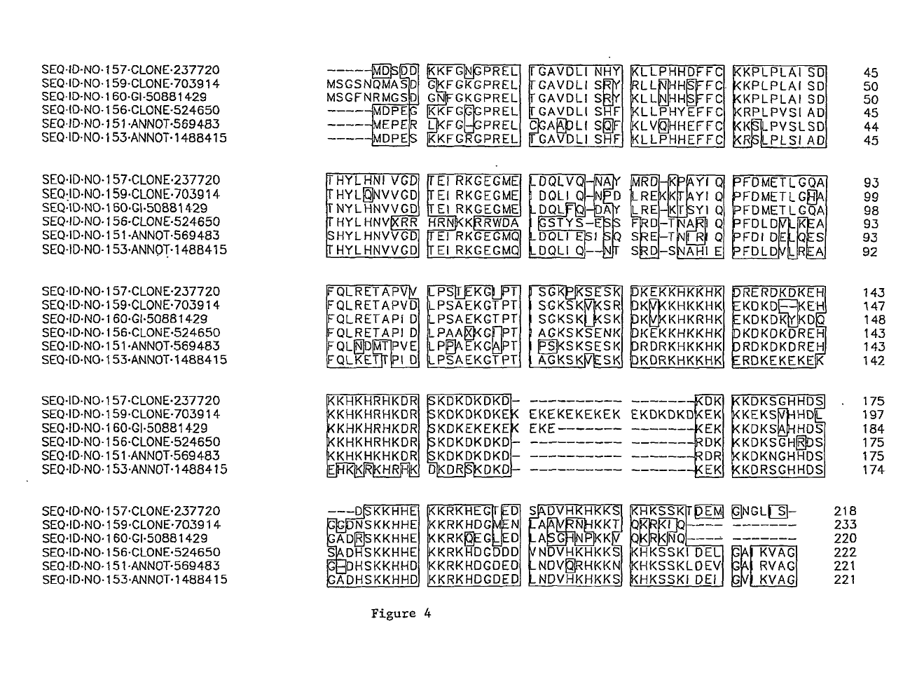

For example, the alignment in FIG. 4 provides the amino acid sequences of Annot 569483 (SEQ ID NO:151), Annot 1488415 (SEQ ID NO:153), Clone 524650 (SEQ ID NO:156), Clone 237720 (SEQ ID NO:157), Clone 703914 (SEQ ID NO:159), and gi|150881429 (SEQ ID NO:160). Other homologs and/or orthologs include Ceres ANNOT ID no. 1460393 (SEQ ID NO:155), Ceres CLONE ID no. 465517 (SEQ ID NO:158), Ceres CLONE ID no. 1817099 (SEQ ID NO:348), Ceres CLONE ID no. 1808214 (SEQ ID NO:350), Ceres CLONE ID no. 1870041 (SEQ ID NO:352), and Public GI ID no. 108862961 (SEQ ID NO:353).

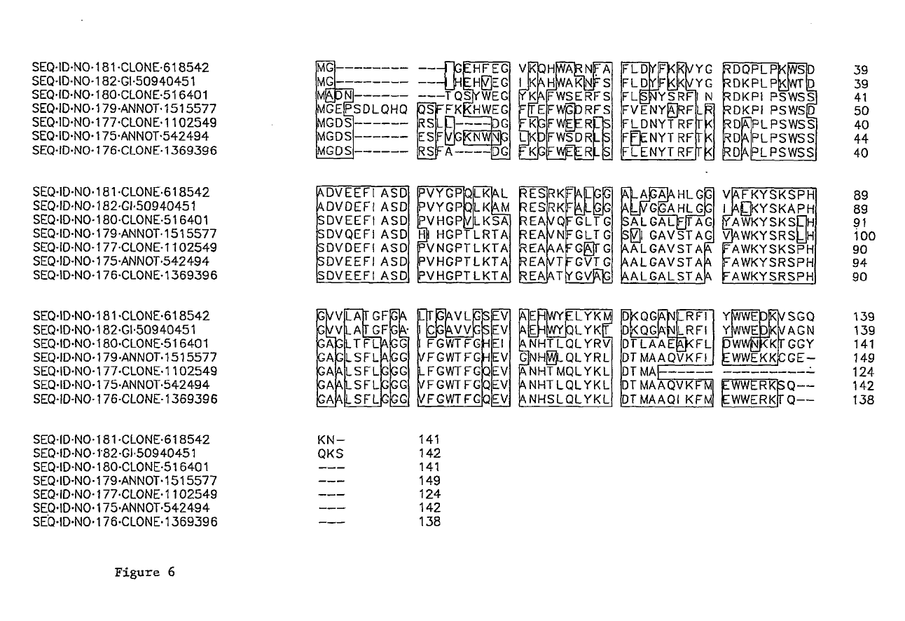

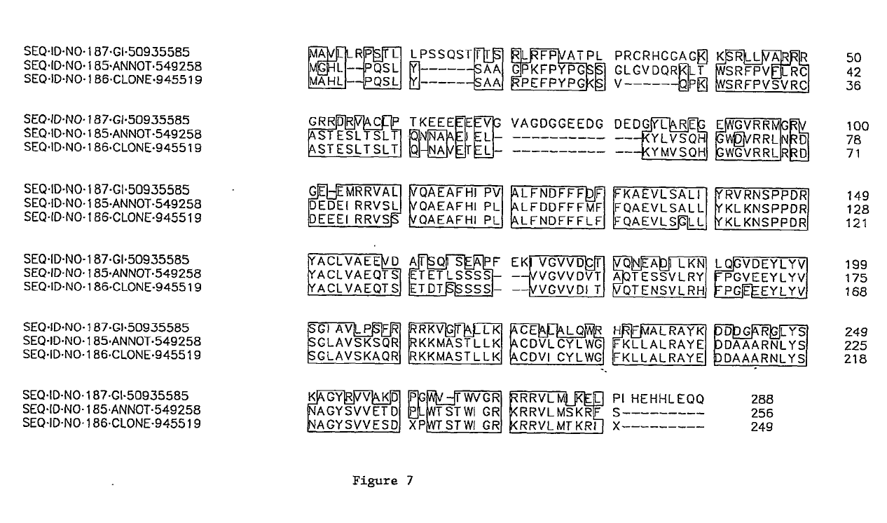

For example, the alignment in FIG. 6 provides the amino acid sequences of Annot 542494 (SEQ ID NO:175), Clone 1369396 (SEQ ID NO:176), Clone 1102549 (SEQ ID NO:177), Annot 1515577 (SEQ ID NO:179), Clone 516401 (SEQ ID NO:180), Clone 618542 (SEQ ID NO:181), and gi|50940451 (SEQ ID NO:182). Other homologs and/or orthologs include Ceres CLONE ID no. 305154 (SEQ ID NO:183) and Ceres CLONE ID no. 1779106 (SEQ ID NO:309).

In some cases, an oil-modulating polypeptide includes a polypeptide having at least 80% sequence identity, e.g., 80%, 85%, 90%, 95%, 97%, 98%, or 99% sequence identity, to an amino acid sequence corresponding to any of SEQ ID NO:153, SEQ ID NO:155, SEQ ID NO:156, SEQ ID NO:157, SEQ ID NO:158, SEQ ID NO:159, SEQ ID NO:160, SEQ ID NO:176, SEQ ID NO:177, SEQ ID NO:179, SEQ ID NO:180, SEQ ID NO:181, SEQ ID NO:182, SEQ ID NO:183, SEQ ID NO:309, SEQ ID NO:348, SEQ ID NO:350, SEQ ID NO:352, SEQ ID NO:353, or SEQ ID NOs:415-441.

An oil-modulating polypeptide encoded by a recombinant nucleic acid can be a native oil-modulating polypeptide, i.e., one or more additional copies of the coding sequence for an oil-modulating polypeptide that is naturally present in the cell. Alternatively, an oil-modulating polypeptide can be heterologous to the cell, e.g., a transgenic Lycopersicon plant can contain the coding sequence for a transcription factor polypeptide from a Glycine plant.

An oil-modulating polypeptide can include additional amino acids that are not involved in oil modulation, and thus can be longer than would otherwise be the case. For example, an oil-modulating polypeptide can include an amino acid sequence that functions as a reporter. Such an oil-modulating polypeptide can be a fusion protein in which a green fluorescent protein (GFP) polypeptide is fused to, e.g., SEQ ID NO: 87, or in which a yellow fluorescent protein (YFP) polypeptide is fused to, e.g., SEQ ID NO:175. In some embodiments, an oil-modulating polypeptide includes a purification tag, a chloroplast transit peptide, a mitochondrial transit peptide, or a leader sequence added to the amino or carboxy terminus.

Oil-modulating polypeptide candidates suitable for use in the invention can be identified by analysis of nucleotide and polypeptide sequence alignments. For example, performing a query on a database of nucleotide or polypeptide sequences can identify homologs and/or orthologs of oil-modulating polypeptides. Sequence analysis can involve BLAST, Reciprocal BLAST, or PSI-BLAST analysis of nonredundant databases using known oil-modulating polypeptide amino acid sequences. Those polypeptides in the database that have greater than 40% sequence identity can be identified as candidates for further evaluation for suitability as an oil-modulating polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains suspected of being present in oil-modulating polypeptides, e.g., conserved functional domains.

The identification of conserved regions in a template or subject polypeptide can facilitate production of variants of wild type oil-modulating polypeptides. Conserved regions can be identified by locating a region within the primary amino acid sequence of a template polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains at sanger.ac.uk/Pfam and genome.wustl.edu/Pfam. A description of the information included at the Pfam database is described in Sonnhammer et al., Nucl. Acids Res., 26:320-322 (1998); Sonnhammer et al., Proteins, 28:405-420 (1997); and Bateman et al., Nucl. Acids Res., 27:260-262 (1999). Amino acid residues corresponding to Pfam domains included in oil-modulating polypeptides provided herein are set forth in the sequence listing. For example, amino acid residues 141 to 205 of the amino acid sequence set forth in SEQ ID NO:82 correspond to an AP2 domain, as indicated in fields <222> and <223> for SEQ ID NO:82 in the sequence listing.

Conserved regions also can be determined by aligning sequences of the same or related polypeptides from closely related species. Closely related species preferably are from the same family. In some embodiments, alignment of sequences from two different species is adequate. For example, sequences from Arabidopsis and Zea mays can be used to identify one or more conserved regions.

Typically, polypeptides that exhibit at least about 40% amino acid sequence identity are useful to identify conserved regions. Conserved regions of related polypeptides can exhibit at least 45% amino acid sequence identity (e.g., at least 50%, at least 60%, at least 70%, at least 80%, or at least 90% amino acid sequence identity). In some embodiments, a conserved region of target and template polypeptides exhibit at least 92%, 94%, 96%, 98%, or 99% amino acid sequence identity. Amino acid sequence identity can be deduced from amino acid or nucleotide sequences. In certain cases, highly conserved domains have been identified within oil-modulating polypeptides. These conserved regions can be useful in identifying functionally similar (orthologous) oil-modulating polypeptides.

In some instances, suitable oil-modulating polypeptides can be synthesized on the basis of consensus functional domains and/or conserved regions in polypeptides that are homologous oil-modulating polypeptides. Domains are groups of substantially contiguous amino acids in a polypeptide that can be used to characterize protein families and/or parts of proteins. Such domains have a “fingerprint” or “signature” that can comprise conserved (1) primary sequence, (2) secondary structure, and/or (3) three-dimensional conformation. Generally, domains are correlated with specific in vitro and/or in vivo activities. A domain can have a length of from 10 amino acids to 400 amino acids, e.g., 10 to 50 amino acids, or 25 to 100 amino acids, or 35 to 65 amino acids, or 35 to 55 amino acids, or 45 to 60 amino acids, or 200 to 300 amino acids, or 300 to 400 amino acids.

Representative homologs and/or orthologs of oil-modulating polypeptides are shown in FIGS. 1-14. Each Figure represents an alignment of the amino acid sequence of an oil-modulating polypeptide with the amino acid sequences of corresponding homologs and/or orthologs. Amino acid sequences of oil-modulating polypeptides and their corresponding homologs and/or orthologs have been aligned to identify conserved amino acids, as shown in FIGS. 1-14. A dash in an aligned sequence represents a gap, i.e., a lack of an amino acid at that position. Identical amino acids or conserved amino acid substitutions among aligned sequences are identified by boxes. Each conserved region contains a sequence of contiguous amino acid residues.

Useful polypeptides can be constructed based on the conserved regions in FIG. 1, FIG. 2, FIG. 3, FIG. 4, FIG. 5, FIG. 6, FIG. 7, FIG. 8, FIG. 9, FIG. 10, FIG. 11, FIG. 12, FIG. 13, or FIG. 14. Such a polypeptide includes the conserved regions, arranged in the order depicted in the Figure from amino-terminal end to carboxy-terminal end. Such a polypeptide may also include zero, one, or more than one amino acid in positions marked by dashes. When no amino acids are present at positions marked by dashes, the length of such a polypeptide is the sum of the amino acid residues in all conserved regions. When amino acids are present at all positions marked by dashes, such a polypeptide has a length that is the sum of the amino acid residues in all conserved regions and all dashes.

Conserved regions can be identified by homologous polypeptide sequence analysis as described above. The suitability of polypeptides for use as oil-modulating polypeptides can be evaluated by functional complementation studies.

Useful polypeptides can also be identified based on the polypeptides set forth in any of FIGS. 1-14 using algorithms designated as Hidden Markov Models. A “Hidden Markov Model (HMM)” is a statistical model of a consensus sequence for a group of homologous and/or orthologous polypeptides. See, Durbin et al., Biological Sequence Analysis Probabilistic Models of Proteins and Nucleic Acids, Cambridge University Press, Cambridge, UK.(1998). An HMM is generated by the program HMMER 2.3.2 using the multiple sequence alignment of the group of homologous and/or orthologous sequences as input and the default program parameters. The multiple sequence alignment is generated by ProbCons (Do et al., Genome Res., 15(2):330-40 (2005)) version 1.11 using a set of default parameters: -c, --consistency REPS of 2; -ir, --iterative-refinement REPS of 100; -pre, --pre-training REPS of 0. ProbCons is a public domain software program provided by Stanford University.

The default parameters for building an HMM (hmmbuild) are as follows: the default “architecture prior” (archpri) used by MAP architecture construction is 0.85, and the default cutoff threshold (idlevel) used to determine the effective sequence number is 0.62. The HMMER 2.3.2 package was released Oct. 3, 2003 under a GNU general public license, and is available from various sources on the World Wide Web such as hmmer.janelia.org, hmmer.wustl.edu, and fr.com/hmmer232/. Hmmbuild outputs the model as a text file.

The HMM for a group of homologous and/or orthologous polypeptides can be used to determine the likelihood that a subject polypeptide sequence is a better fit to that particular HMM than to a null HMM generated using a group of sequences that are not homologous and/or orthologous. The likelihood that a subject polypeptide sequence is a better fit to an HMM than to a null HMM is indicated by the HMM bit score, a number generated when the subject sequence is fitted to the HMM profile using the HMMER hmmsearch program. The following default parameters are used when running hmmsearch: the default E-value cutoff (E) is 10.0, the default bit score cutoff (T) is negative infinity, the default number of sequences in a database (Z) is the real number of sequences in the database, the default E-value cutoff for the per-domain ranked hit list (domE) is infinity, and the default bit score cutoff for the per-domain ranked hit list (domT) is negative infinity. A high HMM bit score indicates a greater likelihood that the subject sequence carries out one or more of the biochemical or physiological function(s) of the polypeptides used to generate the HMM. A high HMM bit score is at least 20, and often is higher.

An oil-modulating polypeptide can fit an HMM provided herein with an HMM bit score greater than 20 (e.g., greater than 30, 40, 50, 60, 70, 80, 90, 100, 200, 300, 400, or 500). In some cases, an oil-modulating polypeptide can fit an HMM provided herein with an HMM bit score that is about 50%, 60%, 70%, 80%, 90%, or 95% of the HMM bit score of any homologous and/or orthologous polypeptide provided in any of Tables 27-40. In some cases, an oil-modulating polypeptide can fit an HMM described herein with an HMM bit score greater than 20, and can have a conserved domain, e.g., a PFAM domain, or a conserved region having 70% or greater sequence identity (e.g., 75%, 80%, 85%, 90%, 95%, or 100% sequence identity) to a conserved domain or region present in an oil-modulating polypeptide disclosed herein.

For example, an oil-modulating polypeptide can fit an HMM generated using the amino acid sequences set forth in FIG. 1 with an HMM bit score that is greater than about 200 (e.g., greater than about 250, 300, 350, 400, 450, 500, 550, 600, 650, or 700). In some cases, an oil-modulating polypeptide can fit an HMM generated using the amino acid sequences set forth in FIG. 2 with an HMM bit score that is greater than about 250 (e.g., greater than about 300, 350, 400, 450, 500, 550, 600, 650, 700, 750, or 800). In some cases, an oil modulating polypeptide can fit an HMM generated using the amino acid sequences set forth in FIG. 3 with an HMM bit score that is greater than about 200 (e.g., greater than about 250, 275, 300, 325, 350, 375, 400, 425, or 450). In some cases, an oil-modulating polypeptide can fit an HMM generated using the amino acid sequences set forth in FIG. 4 with an HMM bit score that is greater than about 150 (e.g., greater than about 175, 200, 225, 250, 275, 300, 325, 350, 375, 400, 425, or 450). In some cases, an oil-modulating polypeptide can fit an HMM generated using the amino acid sequences set forth in FIG. 5 with an HMM bit score that is greater than about 150 (e.g., greater than about 175, 200, 225, 250, 275, 300, 325, 350, 375, or 400). In some cases, an oil-modulating polypeptide can fit an HMM generated using the amino acid sequences set forth in FIG. 6 with an HMM bit score that is greater than about 145 (e.g., greater than about 150, 175, 200, 225, 250, 275, 300, 325, or 350). In some cases, an oil-modulating polypeptide can fit an HMM generated using the amino acid sequences set forth in FIG. 7 with an HMM bit score that is greater than about 350 (e.g., greater than about 400, 450, 500, 550, 600, 650, 700, or 750). In some cases, an oil-modulating polypeptide can fit an HMM generated using the amino acid sequences set forth in FIG. 8 with an HMM bit score that is greater than about 300 (e.g., greater than about 350, 400, 450, 500, 550, 600, or 650). In some cases, an oil-modulating polypeptide can fit an HMM generated using the amino acid sequences set forth in FIG. 9 with an HMM bit score that is greater than about 250 (e.g., greater than about 275, 300, 325, 350, 375, 400, 425, 450, 475, 500, 525, 550, 575, or 600). In some cases, an oil-modulating polypeptide can fit an HMM generated using the amino acid sequences set forth in FIG. 10 with an HMM bit score that is greater than about 50 (e.g., greater than about 75, 80, 90, 100, 125, 150, 175, 200, 225, 250, 275, or 300). In some cases, an oil-modulating polypeptide can fit an HMM generated using the amino acid sequences set forth in FIG. 11 with an HMM bit score that is greater than about 50 (e.g., greater than about 55, 60, 65, 70, 75, 80, 85, 90, 95, 100, 110, 120, 130, 140, 150, 160, or 170). In some cases, an oil-modulating polypeptide can fit an HMM generated using the amino acid sequences set forth in FIG. 12 with an HMM bit score that is greater than about 450 (e.g., greater than about 475, 500, 550, 600, 650, 700, 750, 800, 850, 900, 950, 1000, 1050, or 1100). In some cases, an oil-modulating polypeptide can fit an HMM generated using the amino acid sequences set forth in FIG. 13 with an HMM bit score that is greater than about 500 (e.g., greater than about 550, 600, 650, 700, 750, 800, 850, 900, 950, or 1000). In some cases, an oil-modulating polypeptide can fit an HMM generated using the amino acid sequences set forth in FIG. 14 with an HMM bit score that is greater than about 50 (e.g., greater than about 60, 65, 70, 75, 80, 85, 90, 95, 100, 105, 110, 115, 120, 125, 130, 135, 140, or 145).

### Nucleic Acids

The terms “nucleic acid” and “polynucleotide” are used interchangeably herein, and refer to both RNA and DNA, including cDNA, genomic DNA, synthetic DNA, and DNA (or RNA) containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA), transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

Nucleic acids described herein include oil-modulating nucleic acids. Oil-modulating nucleic acids can be effective to modulate oil levels when transcribed in a plant or plant cell. An oil-modulating nucleic acid can comprise the nucleotide sequence set forth in SEQ ID NO:79. Alternatively, an oil-modulating nucleic acid can be a variant of the nucleic acid having the nucleotide sequence set forth in SEQ ID NO:79. For example, an oil-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:79.

An “isolated” nucleic acid can be, for example, a naturally-occurring DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule, independent of other sequences (e.g., a chemically synthesized nucleic acid, or a cDNA or genomic DNA fragment produced by the polymerase chain reaction (PCR) or restriction endonuclease treatment). An isolated nucleic acid also refers to a DNA molecule that is incorporated into a vector, an autonomously replicating plasmid, a virus, or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

Isolated nucleic acid molecules can be produced by standard techniques. For example, polymerase chain reaction (PCR) techniques can be used to obtain an isolated nucleic acid containing a nucleotide sequence described herein. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid. Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids of the invention also can be obtained by mutagenesis of, e.g., a naturally occurring DNA.

As used herein, the term “percent sequence identity” refers to the degree of identity between any given query sequence and a subject sequence. A subject sequence typically has a length that is more than 80 percent, e.g., more than 82, 85, 87, 89, 90, 93, 95, 97, 99, 100, 105, 110, 115, or 120 percent, of the length of the query sequence. A query nucleic acid or amino acid sequence is aligned to one or more subject nucleic acid or amino acid sequences using the computer program ClustalW (version 1.83, default parameters), which allows alignments of nucleic acid or protein sequences to be carried out across their entire length (global alignment). Chema et al., Nucleic Acids Res., 31(13):3497-500 (2003).

ClustalW calculates the best match between a query and one or more subject sequences, and aligns them so that identities, similarities and differences can be determined. Gaps of one or more residues can be inserted into a query sequence, a subject sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The output is a sequence alignment that reflects the relationship between sequences. ClustalW can be run, for example, at the Baylor College of Medicine Search Launcher site (searchlauncher.bcm.tmc.edu/multi-align/multi-align.html) and at the European Bioinformatics Institute site on the World Wide Web (ebi.ac.uk/clustalw).

To determine a percent identity between a query sequence and a subject sequence, ClustalW divides the number of identities in the best alignment by the number of residues compared (gap positions are excluded), and multiplies the result by 100. The output is the percent identity of the subject sequence with respect to the query sequence. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

The term “exogenous” with respect to a nucleic acid indicates that the nucleic acid is part of a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. It will be appreciated that an exogenous nucleic acid may have been introduced into a progenitor and not into the cell under consideration. For example, a transgenic plant containing an exogenous nucleic acid can be the progeny of a cross between a stably transformed plant and a non-transgenic plant. Such progeny are considered to contain the exogenous nucleic acid.

Recombinant constructs are also provided herein and can be used to transform plants or plant cells in order to modulate oil levels. A recombinant nucleic acid construct comprises a nucleic acid encoding an oil-modulating polypeptide as described herein, operably linked to a regulatory region suitable for expressing the oil-modulating polypeptide in the plant or cell. Thus, a nucleic acid can comprise a coding sequence that encodes any of the oil-modulating polypeptides as set forth in SEQ ID NO:80, SEQ ID NOs:82-85, SEQ ID NOs:87-127, SEQ ID NOs:129-130, SEQ ID NOs:132-133, SEQ ID NOs:135-138, SEQ ID NOs:140-146, SEQ ID NOs:148-149, SEQ ID NO:151, SEQ ID NO:153, SEQ ID NOs:155-160, SEQ ID NOs:162-164, SEQ ID NO:166, SEQ ID NOs:168-171, SEQ ID NO:173, SEQ ID NOs:175-177, SEQ ID NOs:179-183, SEQ ID NOs:185-188, SEQ ID NOs:190-193, SEQ ID NOs:195-196, SEQ ID NOs:198-199, SEQ ID NO:201, SEQ ID NOs:203-214, SEQ ID NO:216, SEQ ID NO:218, SEQ ID NOs:220-227, SEQ ID NOs:229-230, SEQ ID NOs:232-235, SEQ ID NOs:237-243, SEQ ID NO:245, SEQ ID NOs:247-249, SEQ ID NO:309, SEQ ID NO:311, SEQ ID NO:313, SEQ ID NO:315, SEQ ID NOs:317-318, SEQ ID NOs:320-321, SEQ ID NO:323, SEQ ID NO:325, SEQ ID NO:327, SEQ ID NO:329, SEQ ID NOs:331-332, SEQ ID NOs:334-335, SEQ ID NOs:337-338, SEQ ID NOs:340-341, SEQ ID NOs:343-344, SEQ ID NO:346, SEQ ID NO:348, SEQ ID NO:350, SEQ ID NOs:352-353, SEQ ID NO:355, SEQ ID NO:357, SEQ ID NOs:359-360, SEQ ID NO:367, or SEQ ID NOs:415-441.

Examples of nucleic acids encoding oil-modulating polypeptides are set forth in SEQ ID NO:79, SEQ ID NO:81, SEQ ID NO:86, SEQ ID NO:128, SEQ ID NO:131, SEQ ID NO:134, SEQ ID NO:139, SEQ ID NO:147, SEQ ID NO:150, SEQ ID NO:152, SEQ ID NO:154, SEQ ID NO:161, SEQ ID NO:165, SEQ ID NO:167, SEQ ID NO:172, SEQ ID NO:174, SEQ ID NO:178, SEQ ID NO:184, SEQ ID NO:189, SEQ ID NO:194, SEQ ID NO:197, SEQ ID NO:200, SEQ ID NO:202, SEQ ID NO:215, SEQ ID NO:217, SEQ ID NO:219, SEQ ID NO:228, SEQ ID NO:231, SEQ ID NO:236, SEQ ID NO:244, SEQ ID NO:246, SEQ ID NOs:265-308, SEQ ID NO:310, SEQ ID NO:312, SEQ ID NO:314, SEQ ID NO:316, SEQ ID NO:319, SEQ ID NO:322, SEQ ID NO:324, SEQ ID NO:326, SEQ ID NO:328, SEQ ID NO:330, SEQ ID NO:333, SEQ ID NO:336, SEQ ID NO:339, SEQ ID NO:342, SEQ ID NO:345, SEQ ID NO:347, SEQ ID NO:349, SEQ ID NO:351, SEQ ID NO:354, SEQ ID NO:356, SEQ ID NO:358, and SEQ ID NOs:361-366.

In some cases, a recombinant nucleic acid construct can include a nucleic acid comprising less than the full-length coding sequence of an oil-modulating polypeptide. In some cases, a recombinant nucleic acid construct can include a nucleic acid comprising a coding sequence, a gene, or a fragment of a coding sequence or gene in an antisense orientation so that the antisense strand of RNA is transcribed.

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid. For example, codons in the coding sequence for a given oil-modulating polypeptide can be modified such that optimal expression in a particular plant species is obtained, using appropriate codon bias tables for that species.

Vectors containing nucleic acids such as those described herein also are provided. A “vector” is a replicon, such as a plasmid, phage, or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes a regulatory region. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

The vectors provided herein also can include, for example, origins of replication, scaffold attachment regions (SARs), and/or markers. A marker gene can confer a selectable phenotype on a plant cell. For example, a marker can confer biocide resistance, such as resistance to an antibiotic (e.g., kanamycin, G418, bleomycin, or hygromycin), or an herbicide (e.g., chlorosulfuron or phosphinothricin). In addition, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

### Regulatory Regions

The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns.

As used herein, the term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site, or about 2,000 nucleotides upstream of the transcription start site. A promoter typically comprises at least a core (basal) promoter. A promoter also may include at least one control element, such as an enhancer sequence, an upstream element or an upstream activation region (UAR). For example, a suitable enhancer is a cis-regulatory element (−212 to −154) from the upstream region of the octopine synthase (ocs) gene. Fromm et al., The Plant Cell, 1:977-984 (1989). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Some suitable promoters initiate transcription only, or predominantly, in certain cell types. For example, a promoter that is active predominantly in a reproductive tissue (e.g., fruit, ovule, pollen, pistils, female gametophyte, egg cell, central cell, nucellus, suspensor, synergid cell, flowers, embryonic tissue, embryo sac, embryo, zygote, endospern, integument, or seed coat) can be used. Thus, as used herein a cell type- or tissue-preferential promoter is one that drives expression preferentially in the target tissue, but may also lead to some expression in other cell types or tissues as well. Methods for identifying and characterizing promoter regions in plant genomic DNA include, for example, those described in the following references: Jordano et al., Plant Cell, 1:855-866 (1989); Bustos et al., Plant Cell, 1:839-854 (1989); Green et al., EMBO J., 7:4035-4044 (1988); Meier et al., Plant Cell, 3:309-316 (1991); and Zhang et al., Plant Physiology, 110:1069-1079 (1996).

Examples of various classes of promoters are described below. Some of the promoters indicated below as well as additional promoters are described in more detail in U.S. patent application Ser. Nos. 60/505,689; 60/518,075; 60/544,771; 60/558,869; 60/583,691; 60/619,181; 60/637,140; 60/757,544; 60/776,307; 10/957,569; 11/058,689; 11/172,703; 11/208,308; 11/274,890; 60/583,609; 60/612,891; 11/097,589; 11/233,726; 10/950,321; PCT/US05/011105; PCT/US05/034308; and PCT/US05/23639. Nucleotide sequences of promoters are set forth in SEQ ID NOs:1-78 and SEQ ID NOs:250-264. It will be appreciated that a promoter may meet criteria for one classification based on its activity in one plant species, and yet meet criteria for a different classification based on its activity in another plant species.

Broadly Expressing Promoters

A promoter can be said to be “broadly expressing” when it promotes transcription in many, but not necessarily all, plant tissues. For example, a broadly expressing promoter can promote transcription of an operably linked sequence in one or more of the shoot, shoot tip (apex), and leaves, but weakly or not at all in tissues such as roots or stems. As another example, a broadly expressing promoter can promote transcription of an operably linked sequence in one or more of the stem, shoot, shoot tip (apex), and leaves, but can promote transcription weakly or not at all in tissues such as reproductive tissues of flowers and developing seeds. Non-limiting examples of broadly expressing promoters that can be included in the nucleic acid constructs provided herein include the p326 (SEQ ID NO:76), YP0144 (SEQ ID NO:55), YP0190 (SEQ ID NO:59), p13879 (SEQ ID NO:75), YP0050 (SEQ ID NO:35), p32449 (SEQ ID NO:77), 21876 (SEQ ID NO:1), YP0158 (SEQ ID NO:57), YP0214 (SEQ ID NO:61), YP0380 (SEQ ID NO:70), PT0848 (SEQ ID NO:26), and PT0633 (SEQ ID NO:7) promoters. Additional examples include the cauliflower mosaic virus (CaMV) 35S promoter, the mannopine synthase (MAS) promoter, the 1′ or 2′ promoters derived from T-DNA of Agrobacterium tumefaciens, the figwort mosaic virus 34S promoter, actin promoters such as the rice actin promoter, and ubiquitin promoters such as the maize ubiquitin-1 promoter. In some cases, the CaMV 35S promoter is excluded from the category of broadly expressing promoters.

Root Promoters

Root-active promoters confer transcription in root tissue, e.g., root endodermis, root epidermis, or root vascular tissues. In some embodiments, root-active promoters are root-preferential promoters, i.e., confer transcription only or predominantly in root tissue. Root-preferential promoters include the YP0128 (SEQ ID NO:52), YP0275 (SEQ ID NO:63), PT0625 (SEQ ID NO:6), PT0660 (SEQ ID NO:9), PT0683 (SEQ ID NO:14), and PT0758 (SEQ ID NO:22) promoters. Other root-preferential promoters include the PT0613 (SEQ ID NO:5), PT0672 (SEQ ID NO:11), PT0688 (SEQ ID NO:15), and PT0837 (SEQ ID NO:24) promoters, which drive transcription primarily in root tissue and to a lesser extent in ovules and/or seeds. Other examples of root-preferential promoters include the root-specific subdomains of the CaMV 35S promoter (Lam et al., Proc. Natl. Acad. Sci. USA, 86:7890-7894 (1989)), root cell specific promoters reported by Conkling et al., Plant Physiol., 93:1203-1211 (1990), and the tobacco RD2 promoter.

Maturing Endosperm Promoters

In some embodiments, promoters that drive transcription in maturing endosperm can be useful. Transcription from a maturing endosperm promoter typically begins after fertilization and occurs primarily in endosperm tissue during seed development and is typically highest during the cellularization phase. Most suitable are promoters that are active predominantly in maturing endosperm, although promoters that are also active in other tissues can sometimes be used. Non-limiting examples of maturing endosperm promoters that can be included in the nucleic acid constructs provided herein include the napin promoter, the Arcelin-5 promoter, the phaseolin promoter (Bustos et al., Plant Cell, 1(9):839-853 (1989)), the soybean trypsin inhibitor promoter (Riggs et al., Plant Cell, 1(6):609-621 (1989)), the ACP promoter (Baerson et al., Plant Mol. Biol., 22(2):255-267 (1993)), the stearoyl-ACP desaturase promoter (Slocombe et al., Plant Physiol., 104(4):167-176 (1994)), the soybean α′ subunit of β-conglycinin promoter (Chen et al., Proc. Natl. Acad. Sci. USA, 83:8560-8564 (1986)), the oleosin promoter (Hong et al., Plant Mol. Biol., 34(3):549-555 (1997)), and zein promoters, such as the 15 kD zein promoter, the 16 kD zein promoter, 19 kD zein promoter, 22 kD zein promoter and 27 kD zein promoter. Also suitable are the Osgt-1 promoter from the rice glutelin-1 gene (Zheng et al., Mol. Cell. Biol., 13:5829-5842 (1993)), the beta-amylase promoter, and the barley hordein promoter. Other maturing endosperm promoters include the YP0092 (SEQ ID NO:38), PT0676 (SEQ ID NO:12), and PT0708 (SEQ ID NO:17) promoters.

Ovary Tissue Promoters

Promoters that are active in ovary tissues such as the ovule wall and mesocarp can also be useful, e.g., a polygalacturonidase promoter, the banana TRX promoter, and the melon actin promoter. Examples of promoters that are active primarily in ovules include YP0007 (SEQ ID NO:30), YP0111(SEQ ID NO:46), YP0092 (SEQ ID NO:38), YP0103 (SEQ ID NO:43), YP0028 (SEQ ID NO:33), YP6121 (SEQ ID NO:51), YP0008 (SEQ ID NO:31), YP0039 (SEQ ID NO:34), YP0115 (SEQ ID NO:47), YP0119 (SEQ ID NO:49), YP0120 (SEQ ID NO:50), and YP0374 (SEQ ID NO:68).

Embryo Sac/Early Endosperm Promoters

To achieve expression in embryo sac/early endosperm, regulatory regions can be used that are active in polar nuclei and/or the central cell, or in precursors to polar nuclei, but not in egg cells or precursors to egg cells. Most suitable are promoters that drive expression only or predominantly in polar nuclei or precursors thereto and/or the central cell. A pattern of transcription that extends from polar nuclei into early endosperm development can also be found with embryo sac/early endosperm-preferential promoters, although transcription typically decreases significantly in later endosperm development during and after the cellularization phase. Expression in the zygote or developing embryo typically is not present with embryo sac/early endosperm promoters.

Promoters that may be suitable include those derived from the following genes: Arabidopsis viviparous-1 (see, GenBank No. U93215); Arabidopsis atmycl (see, Urao (1996) Plant Mol. Biol., 32:571-57; Conceicao (1994) Plant, 5:493-505); Arabidopsis FIE (GenBank No. AF129516); Arabidopsis MEA; Arabidopsis FIS2 (GenBank No. AF096096); and FIE 1.1 (U.S. Pat. No. 6,906,244). Other promoters that may be suitable include those derived from the following genes: maize MAC1 (see, Sheridan (1996) Genetics, 142:1009-1020); maize Cat3 (see, GenBank No. L05934; Abler (1993) Plant Mol. Biol., 22:10131-1038). Other promoters include the following Arabidopsis promoters: YP0039 (SEQ ID NO:34), YP0101 (SEQ ID NO:41), YP0102 (SEQ ID NO:42), YP0110 (SEQ ID NO:45), YP0117 (SEQ ID NO:48), YP0119 (SEQ ID NO:49), YP0137 (SEQ ID NO:53), DME, YP0285 (SEQ ID NO:64), and YP0212 (SEQ ID NO:60). Other promoters that may be useful include the following rice promoters: p530c10 (SEQ ID NO:250), pOsFIE2-2 (SEQ ID NO:251), pOsMEA (SEQ ID NO:252), pOsYp102 (SEQ ID NO:253), and pOsYp285 (SEQ ID NO:254).

Embryo Promoters

Regulatory regions that preferentially drive transcription in zygotic cells following fertilization can provide embryo-preferential expression. Most suitable are promoters that preferentially drive transcription in early stage embryos prior to the heart stage, but expression in late stage and maturing embryos is also suitable. Embryo-preferential promoters include the barley lipid transfer protein (Ltp1) promoter (Plant Cell Rep (2001) 20:647-654), YP0097 (SEQ ID NO:40), YP0107 (SEQ ID NO:44), YP0088 (SEQ ID NO:37), YP0143 (SEQ ID NO:54), YP0156 (SEQ ID NO:56), PT0650 (SEQ ID NO:8), PT0695 (SEQ ID NO:16), PT0723 (SEQ ID NO:19), PT0838 (SEQ ID NO:25), PT0879 (SEQ ID NO:28), and PT0740 (SEQ ID NO:20).

Photosynthetic Tissue Promoters

Promoters active in photosynthetic tissue confer transcription in green tissues such as leaves and stems. Most suitable are promoters that drive expression only or predominantly in such tissues. Examples of such promoters include the ribulose-1,5-bisphosphate carboxylase (RbcS) promoters such as the RbcS promoter from eastern larch (Larix laricina), the pine cab6 promoter (Yamamoto et al., Plant Cell Physiol., 35:773-778 (1994)), the Cab-1 promoter from wheat (Fejes et al., Plant Mol. Biol., 15:921-932 (1990)), the CAB-1 promoter from spinach (Lubberstedt et al., Plant Physiol., 104:997-1006 (1994)), the cab1R promoter from rice (Luan et al., Plant Cell, 4:971-981 (1992)), the pyruvate orthophosphate dikinase (PPDK) promoter from corn (Matsuoka et al., Proc. Natl. Acad. Sci. USA, 90:9586-9596 (1993)), the tobacco Lhcb1*2 promoter (Cerdan et al., Plant Mol. Biol., 33:245-255 (1997)), the Arabidopsis thaliana SUC2 sucrose-H+ symporter promoter (Truemit et al., Planta, 196:564-570 (1995)), and thylakoid membrane protein promoters from spinach (psaD, psaF, psaE, PC, FNR, atpC, atpD, cab, rbcS). Other photosynthetic tissue promoters include PT0535 (SEQ ID NO:3), PT0668 (SEQ ID NO:2), PT0886 (SEQ ID NO:29), YP0144 (SEQ ID NO:55), YP0380 (SEQ ID NO:70), and PT0585 (SEQ ID NO:4).

Vascular Tissue Promoters

Examples of promoters that have high or preferential activity in vascular bundles include YP0087 (SEQ ID NO:257), YP0093 (SEQ ID NO:258), YP0108 (SEQ ID NO:259), YP0022 (SEQ ID NO:260), and YP0080 (SEQ ID NO:261). Other vascular tissue-preferential promoters include the glycine-rich cell wall protein GRP 1.8 promoter (Keller and Baumgartner, Plant Cell, 3(10):1051-1061 (1991)), the Commelina yellow mottle virus (CoYMV) promoter (Medberry et al., Plant Cell, 4(2):185-192 (1992)), and the rice tungro bacilliform virus (RTBV) promoter (Dai et al., Proc. Natl. Acad. Sci. USA, 101(2):687-692 (2004)).

Inducible Promoters

Inducible promoters confer transcription in response to external stimuli such as chemical agents or environmental stimuli. For example, inducible promoters can confer transcription in response to hormones such as giberellic acid or ethylene, or in response to light or drought. Examples of drought-inducible promoters include YP0380 (SEQ ID NO:70), PT0848 (SEQ ID NO:26), YP0381 (SEQ ID NO:71), YP0337 (SEQ ID NO:66), PT0633 (SEQ ID NO:7), YP0374 (SEQ ID NO:68), PT0710 (SEQ ID NO:18), YP0356 (SEQ ID NO:67), YP0385 (SEQ ID NO:73), YP0396 (SEQ ID NO:74), YP0388 (SEQ ID NO:262), YP0384 (SEQ ID NO:72), PT0688 (SEQ ID NO:15), YP0286 (SEQ ID NO:65), YP0377 (SEQ ID NO:69), PD1367 (SEQ ID NO:78), PD0901 (SEQ ID NO:263), and PD0898. Nitrogen-inducible promoters include PT0863 (SEQ ID NO:27), PT0829 (SEQ ID NO:23), PT0665 (SEQ ID NO:10), and PT0886 (SEQ ID NO:29).

Basal Promoters

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

Other Promoters

Other classes of promoters include, but are not limited to, leaf-preferential, stem/shoot-preferential, callus-preferential, guard cell-preferential, such as PT0678 (SEQ ID NO:13), and senescence-preferential promoters. Promoters designated YP0086 (SEQ ID NO:36), YP0188 (SEQ ID NO:58), YP0263 (SEQ ID NO:62), PT0758 (SEQ ID NO:22), PT0743 (SEQ ID NO:21), PT0829 (SEQ ID NO:23), YP0119 (SEQ ID NO:49), and YP0096 (SEQ ID NO:39), as described in the above-referenced patent applications, may also be useful.

Other Regulatory Regions

A 5′ untranslated region (UTR) can be included in nucleic acid constructs described herein. A 5′ UTR is transcribed, but is not translated, and lies between the start site of the transcript and the translation initiation codon and may include the +1 nucleotide. A 3′ UTR can be positioned between the translation termination codon and the end of the transcript. UTRs can have particular functions such as increasing mRNA stability or attenuating translation. Examples of 3′ UTRs include, but are not limited to, polyadenylation signals and transcription termination sequences, e.g., a nopaline synthase termination sequence.

It will be understood that more than one regulatory region may be present in a recombinant polynucleotide, e.g., introns, enhancers, upstream activation regions, transcription terminators, and inducible elements. Thus, more than one regulatory region can be operably linked to the sequence of a polynucleotide encoding an oil-modulating polypeptide.

Regulatory regions, such as promoters for endogenous genes, can be obtained by chemical synthesis or by subcloning from a genomic DNA that includes such a regulatory region. A nucleic acid comprising such a regulatory region can also include flanking sequences that contain restriction enzyme sites that facilitate subsequent manipulation.

Transgenic Plants and Plant Cells

The invention also features transgenic plant cells and plants comprising at least one recombinant nucleic acid construct described herein. A plant or plant cell can be transformed by having a construct integrated into its genome, i.e., can be stably transformed. Stably transformed cells typically retain the introduced nucleic acid with each cell division. A plant or plant cell can also be transiently transformed such that the construct is not integrated into its genome. Transiently transformed cells typically lose all or some portion of the introduced nucleic acid construct with each cell division such that the introduced nucleic acid cannot be detected in daughter cells after a sufficient number of cell divisions. Both transiently transformed and stably transformed transgenic plants and plant cells can be useful in the methods described herein.

Transgenic plant cells used in methods described herein can constitute part or all of a whole plant. Such plants can be grown in a manner suitable for the species under consideration, either in a growth chamber, a greenhouse, or in a field. Transgenic plants can be bred as desired for a particular purpose, e.g., to introduce a recombinant nucleic acid into other lines, to transfer a recombinant nucleic acid to other species, or for further selection of other desirable traits. Alternatively, transgenic plants can be propagated vegetatively for those species amenable to such techniques. As used herein, a transgenic plant also refers to progeny of an initial transgenic plant. Progeny includes descendants of a particular plant or plant line. Progeny of an instant plant include seeds formed on F₁, F₂, F₃, F₄, F₅, F₆ and subsequent generation plants, or seeds formed on BC₁, BC₂, BC₃, and subsequent generation plants, or seeds formed on F₁BC₁, F₁BC₂, F₁BC₃, and subsequent generation plants. The designation F₁ refers to the progeny of a cross between two parents that are genetically distinct. The designations F₂, F₃, F₄, F₅ and F₆ refer to subsequent generations of self- or sib-pollinated progeny of an F₁ plant. Seeds produced by a transgenic plant can be grown and then selfed (or outcrossed and selfed) to obtain seeds homozygous for the nucleic acid construct.

Transgenic plants can be grown in suspension culture, or tissue or organ culture. For the purposes of this invention, solid and/or liquid tissue culture techniques can be used. When using solid medium, transgenic plant cells can be placed directly onto the medium or can be placed onto a filter that is then placed in contact with the medium. When using liquid medium, transgenic plant cells can be placed onto a flotation device, e.g., a porous membrane that contacts the liquid medium. Solid medium typically is made from liquid medium by adding agar. For example, a solid medium can be Murashige and Skoog (MS) medium containing agar and a suitable concentration of an auxin, e.g., 2,4-dichlorophenoxyacetic acid (2,4-D), and a suitable concentration of a cytokinin, e.g., kinetin.

When transiently transformed plant cells are used, a reporter sequence encoding a reporter polypeptide having a reporter activity can be included in the transformation procedure and an assay for reporter activity or expression can be performed at a suitable time after transformation. A suitable time for conducting the assay typically is about 1-21 days after transformation, e.g., about 1-14 days, about 1-7 days, or about 1-3 days. The use of transient assays is particularly convenient for rapid analysis in different species, or to confirm expression of a heterologous oil-modulating polypeptide whose expression has not previously been confirmed in particular recipient cells.

Techniques for introducing nucleic acids into monocotyledonous and dicotyledonous plants are known in the art, and include, without limitation, Agrobacterium-mediated transformation, viral vector-mediated transformation, electroporation and particle gun transformation, e.g., U.S. Pat. Nos. 5,538,880; 5,204,253; 6,329,571 and 6,013,863. If a cell or cultured tissue is used as the recipient tissue for transformation, plants can be regenerated from transformed cultures if desired, by techniques known to those skilled in the art.

Plant Species

The polynucleotides and vectors described herein can be used to transform a number of monocotyledonous and dicotyledonous plants and plant cell systems, including dicots such as alfalfa, almond, amaranth, apple, apricot, avocado, beans (including kidney beans, lima beans, dry beans, green beans), brazil nut, broccoli, cabbage, canola, carrot, cashew, castor bean, cherry, chick peas, chicory, chocolate, clover, cocoa, coffee, cotton, cottonseed, crambe, eucalyptus, flax, grape, grapefruit, hazelnut, hemp, jatropha, jojoba, lemon, lentils, lettuce, linseed, macadamia nut, mango, melon (e.g., watermelon, cantaloupe), mustard, neem, olive, orange, peach, peanut, peach, pear, peas, pecan, pepper, pistachio, plum, poppy, potato, pumpkin, oilseed rape, quinoa, rapeseed (high erucic acid and canola), safflower, sesame, soybean, spinach, strawberry, sugar beet, sunflower, sweet potatoes, tea, tomato, walnut, and yarns, as well as monocots such as banana, barley, bluegrass, coconut, corn, date palm, fescue, field corn, garlic, millet, oat, oil palm, onion, palm kernel oil, pineapple, popcorn, rice, rye, ryegrass, sorghum, sudangrass, sugarcane, sweet corn, switchgrass, turf grasses, timothy, and wheat. Brown seaweeds, green seaweeds, red seaweeds, and microalgae can also be used.

Thus, the methods and compositions described herein can be used with dicotyledonous plants belonging, for example, to the orders Apiales, Arecales, Aristochiales, Asterales, Batales, Campanutales, Capparales, Caryophyllales, Casuarinales, Celastrales, Cornales, Cucurbitales, Diapensales, Dilleniales, Dipsacales, Ebenales, Ericales, Eucomiales, Euphorbiales, Fabales, Fagales, Gentianales, Geraniales, Haloragales, Hamamelidales, illiciales, Juglandales, Lamiales, Laurales, Lecythidales, Leitneriales, Linales, Magniolales, Malvales, Myricales, Myrtales, Nymphaeales, Papaverales, Piperales, Plantaginales, Plumbaginales, Podostemales, Polemoniales, Polygalales, Polygonales, Populus, Primulales, Proteales, Rafflesiales, Ranunculales, Rhamnales, Rosales, Rubiales, Salicales, Santales, Sapindales, Sarraceniaceae, Scrophulariales, Solanales, Trochodendrales, Theales, Umbellales, Urticales, and Violales. The methods and compositions described herein also can be utilized with monocotyledonous plants such as those belonging to the orders Alismatales, Arales, Arecales, Asparagales, Bromeliales, Commelinales, Cyclanthales, Cyperales, Eriocaulales, Hydrocharitales, Juncales, Liliales, Najadales, Orchidales, Pandanales, Poales, Restionales, Triuridales, Typhales, Zingiberales, and with plants belonging to Gymnospermae, e.g., Cycadales, Ginkgoales, Gnetales, and Pinales.

The methods and compositions can be used over a broad range of plant species, including species from the dicot genera Amaranthus, Anacardium, Arachis, Azadirachta, Brassica, Calendula, Camellia, Canarium, Cannabis, Capsicum, Carthamus, Cicer, Cichorium, Cinnamomum, Citrus, Citrullus, Coffea, Corylus, Crambe, Cucumis, Cucurbita, Daucus, Dioscorea, Fragaria, Glycine, Gossypium, Helianthus, Jatropha, Juglans, Lactuca, Lens, Linum, Lycopersicon, Malus, Mangifera, Medicago, Mentha, Nicotiana, Ocimum, Olea, Papaver, Persea, Phaseolus, Pistacia, Pisum, Prunus, Pyrus, Ricinus, Rosmarinus, Salvia, Sesamum, Simmondsia, Solanum, Spinacia, Theobroma, Thymus, Trifolium, Vaccinium, Vigna, and Vitis; and the monocot genera Allium, Ananas, Asparagus, Avena, Cocos, Curcuma, Elaeis, Festuca, Festulolium, Hordeum, Lemna, Lolium, Miscanthus, Musa, Oryza, Panicum, Pennisetum, Phleum, Poa, Saccharum, Secale, Sorghum, Triticosecale, Triticum, and Zea; and the gymnosperm genera Abies, Cunninghamia, Picea, Pinus, and Pseudotsuga.

The methods and compositions described herein also can be used with brown seaweeds, e.g., Ascophyllum nodosum, Fucus vesiculosus, Fucus serratus, Himanthalia elongata, and Undaria pinnatifida; red seaweeds, e.g., Chondrus crispus, Cracilaria verrucosa, Porphyra umbilicalis, and Palmaria palmata; green seaweeds, e.g., Enteromorpha spp. and Ulva spp.; and microalgae, e.g., Spirulina spp. (S. platensis and S. maxima) and Odontella aurita. In addition, the methods and compositions can be used with Crypthecodinium cohnii, Schizochytrium spp., and Haematococcus pluvialis.

In some embodiments, a plant is a member of the species Arachis hypogea, Brassica spp., Carthamus tinctorius, Elaeis oleifera, Glycine max, Gossypium spp., Helianthus annuus, Jatropha curcas, Linum usitatissimum, Miscanthus hybrid (Miscanthus×giganteus), Miscanthus sinensis, Miscanthus sacchariflorus, Panicum virgatum, Populus balsamifera, Saccharum spp., Sorghum bicolor, Triticum aestivum, or Zea mays.

### Methods of Inhibiting Expression of Oil-Modulating Polypeptides

The polynucleotides and recombinant vectors described herein can be used to express or inhibit expression of an oil-modulating polypeptide in a plant species of interest. The term “expression” refers to the process of converting genetic information of a polynucleotide into RNA through transcription, which is catalyzed by an enzyme, RNA polymerase, and into protein, through translation of mRNA on ribosomes. “Up-regulation” or “activation” refers to regulation that increases the production of expression products (mRNA, polypeptide, or both) relative to basal or native states, while “down-regulation” or “repression” refers to regulation that decreases production of expression products (mRNA, polypeptide, or both) relative to basal or native states.

A number of nucleic-acid based methods, including antisense RNA, co-suppression, ribozyme directed RNA cleavage, and RNA interference (RNAi) can be used to inhibit protein expression in plants. Antisense technology is one well-known method. In this method, a nucleic acid segment from a gene to be repressed is cloned and operably linked to a promoter so that the antisense strand of RNA is transcribed. The recombinant vector is then transformed into plants, as described above, and the antisense strand of RNA is produced. The nucleic acid segment need not be the entire sequence of the gene to be repressed, but typically will be substantially complementary to at least a portion of the sense strand of the gene to be repressed. Generally, higher homology can be used to compensate for the use of a shorter sequence. Typically, a sequence of at least 30 nucleotides is used, e.g., at least 40, 50, 80, 100, 200, 500 nucleotides or more.

Thus, for example, an isolated nucleic acid provided herein can be an antisense nucleic acid to any of the aforementioned nucleic acids encoding an oil-modulating polypeptide as set forth in SEQ ID NO:80, SEQ ID NOs:82-85, SEQ ID NOs:87-127, SEQ ID NOs:129-130, SEQ ID NOs:132-133, SEQ ID NOs:135-138, SEQ ID NOs:140-146, SEQ ID NOs:148-149, SEQ ID NO:151, SEQ ID NO:153, SEQ ID NOs:155-160, SEQ ID NOs:162-164, SEQ ID NO:166, SEQ ID NOs:168-171, SEQ ID NO:173, SEQ ID NOs:175-177, SEQ ID NOs:179-183, SEQ ID NOs:185-188, SEQ ID NOs:190-193, SEQ ID NOs:195-196, SEQ ID NOs:198-199, SEQ ID NO:201, SEQ ID NOs:203-214, SEQ ID NO:216, SEQ ID NO:218, SEQ ID NOs:220-227, SEQ ID NOs:229-230, SEQ ID NOs:232-235, SEQ ID NOs:237-243, SEQ ID NO:245, SEQ ID NOs:247-249, SEQ ID NO:309, SEQ ID NO:311, SEQ ID NO:313, SEQ ID NO:315, SEQ ID NOs:317-318, SEQ ID NOs:320-321, SEQ ID NO:323, SEQ ID NO:325, SEQ ID NO:327, SEQ ID NO:329, SEQ ID NOs:331-332, SEQ ID NOs:334-335, SEQ ID NOs:337-338, SEQ ID NOs:340-341, SEQ ID NOs:343-344, SEQ ID NO:346, SEQ ID NO:348, SEQ ID NO:350, SEQ ID NOs:352-353, SEQ ID NO:355, SEQ ID NO:357, SEQ ID NOs:359-360, SEQ ID NO:367, or SEQ ID NOs:415-441. A nucleic acid that decreases the level of a transcription or translation product of a gene encoding an oil-modulating polypeptide is transcribed into an antisense nucleic acid that anneals to the sense coding sequence of the oil-modulating polypeptide.

Constructs containing operably linked nucleic acid molecules in the sense orientation can also be used to inhibit the expression of a gene. The transcription product can be similar or identical to the sense coding sequence of an oil-modulating polypeptide. The transcription product can also be unpolyadenylated, lack a 5′ cap structure, or contain an unsplicable intron. Methods of co-suppression using a full-length cDNA as well as a partial cDNA sequence are known in the art. See, e.g., U.S. Pat. No. 5,231,020.

In another method, a nucleic acid can be transcribed into a ribozyme, or catalytic RNA, that affects expression of an mRNA. (See, U.S. Pat. No. 6,423,885). Ribozymes can be designed to specifically pair with virtually any target mRNA and cleave the phosphodiester backbone at a specific location, thereby functionally inactivating the target RNA. Heterologous nucleic acids can encode ribozymes designed to cleave particular mRNA transcripts, thus preventing expression of a polypeptide. Hammerhead ribozymes are useful for destroying particular mRNAs, although various ribozymes that cleave mRNA at site-specific recognition sequences can be used. Hammerhead ribozymes cleave mRNAs at locations dictated by flanking regions that form complementary base pairs with the target mRNA. The sole requirement is that the target RNA contain a 5′-UG-3′ nucleotide sequence. The construction and production of hammerhead ribozymes is known in the art. See, for example, U.S. Pat. No. 5,254,678 and WO 02/46449 and references cited therein. Hammerhead ribozyme sequences can be embedded in a stable RNA such as a transfer RNA (tRNA) to increase cleavage efficiency in vivo. Perriman et al., Proc. Natl. Acad. Sci. USA, 92(13):6175-6179 (1995); de Feyter and Gaudron, Methods in Molecular Biology, Vol. 74, Chapter 43, “Expressing Ribozyrnes in Plants,” Edited by Turner, P. C., Humana Press Inc., Totowa, N. J. RNA endoribonucleases which have been described, such as the one that occurs naturally in Tetrahymena thermophila, can be useful. See, for example, U.S. Pat. Nos. 4,987,071 and 6,423,885.

RNAi can also be used to inhibit the expression of a gene. For example, a construct can be prepared that includes a sequence that is transcribed into an interfering RNA. Such an RNA can be one that can anneal to itself, e.g., a double stranded RNA having a stem-loop structure. One strand of the stem portion of a double stranded RNA comprises a sequence that is similar or identical to the sense coding sequence of the polypeptide of interest, and that is from about 10 nucleotides to about 2,500 nucleotides in length. The length of the sequence that is similar or identical to the sense coding sequence can be from 10 nucleotides to 500 nucleotides, from 15 nucleotides to 300 nucleotides, from 20 nucleotides to 100 nucleotides, or from 25 nucleotides to 100 nucleotides. The other strand of the stem portion of a double stranded RNA comprises a sequence that is similar or identical to the antisense strand of the coding sequence of the polypeptide of interest, and can have a length that is shorter, the same as, or longer than the corresponding length of the sense sequence. The loop portion of a double stranded RNA can be from 10 nucleotides to 5,000 nucleotides, e.g., from 15 nucleotides to 1,000 nucleotides, from 20 nucleotides to 500 nucleotides, or from 25 nucleotides to 200 nucleotides. The loop portion of the RNA can include an intron. A construct including a sequence that is transcribed into an interfering RNA is transformed into plants as described above. Methods for using RNAi to inhibit the expression of a gene are known to those of skill in the art. See, e.g., U.S. Pat. Nos. 5,034,323; 6,326,527; 6,452,067; 6,573,099; 6,753,139; and 6,777,588. See also WO 97/01952; WO 98/53083; WO 99/32619; WO 98/36083; and U.S. Patent Publications 20030175965, 20030175783, 20040214330, and 20030180945.

In some nucleic-acid based methods for inhibition of gene expression in plants, a suitable nucleic acid can be a nucleic acid analog. Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine and 5-bromo-2′-deoxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six-membered morpholino ring, or peptide nucleic acids, in which the deoxyphosphate backbone is replaced by a pseudopeptide backbone and the four bases are retained. See, for example, Surnmerton and Weller, 1997, Antisense Nucleic Acid Drug Dev., 7:187-195; Hyrup et al., Bioorgan. Med. Chem., 4:5-23 (1996). In addition, the deoxyphosphate backbone can be replaced with, for example, a phosphorothioate or phosphorodithioate backbone, a phosphoroamidite, or an alkyl phosphotriester backbone.

### Transgenic Plant Phenotypes

A transformed cell, callus, tissue, or plant can be identified and isolated by selecting or screening the engineered plant material for particular traits or activities, e.g., expression of a selectable marker gene or modulation of oil content. Such screening and selection methodologies are well known to those having ordinary skill in the art. In addition, physical and biochemical methods can be used to identify transformants. These include Southern analysis or PCR amplification for detection of a polynucleotide; Northern blots, S1 RNase protection, primer-extension, or RT-PCR amplification for detecting RNA transcripts; enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are well known.

A population of transgenic plants can be screened and/or selected for those members of the population that have a desired trait or phenotype conferred by expression of the transgene. Selection and/or screening can be carried out over one or more generations, which can be useful to identify those plants that have a desired trait, such as a modulated level of oil. Selection and/or screening can also be carried out in more than one geographic location. In some cases, transgenic plants can be grown and selected under conditions which induce a desired phenotype or are otherwise necessary to produce a desired phenotype in a transgenic plant. In addition, selection and/or screening can be carried out during a particular developmental stage in which the phenotype is exhibited by the plant.

The phenotype of a transgenic plant can be evaluated relative to a control plant that does not express the exogenous polynucleotide of interest, such as a corresponding wild type plant, a corresponding plant that is not transgenic for the exogenous polynucleotide of interest but otherwise is of the same genetic background as the transgenic plant of interest, or a corresponding plant of the same genetic background in which expression of the polypeptide is suppressed, inhibited, or not induced (e.g., where expression is under the control of an inducible promoter). A plant can be said “not to express” a polypeptide when the plant exhibits less than 10%, e.g., less than 9%, 8%, 7%, 6%, 5%, 4%, 3%, 2%, 1%, 0.5%, 0.1%, 0.01%, or 0.001%, of the amount of polypeptide or mRNA encoding the polypeptide exhibited by the plant of interest. Expression can be evaluated using methods including, for example, RT-PCR, Northern blots, S1 RNase protection, primer extensions, Western blots, protein gel electrophoresis, immunoprecipitation, enzyme-linked immunoassays, chip assays, and mass spectrometry. It should be noted that if a polypeptide is expressed under the control of a tissue-preferential or broadly expressing promoter, expression can be evaluated in the entire plant or in a selected tissue. Similarly, if a polypeptide is expressed at a particular time, e.g., at a particular time in development or upon induction, expression can be evaluated selectively at a desired time period.

In some embodiments, a plant in which expression of an oil-modulating polypeptide is modulated can have increased levels of seed oil. For example, an oil-modulating polypeptide described herein can be expressed in a transgenic plant, resulting in increased levels of seed oil. The seed oil level can be increased by at least 2 percent, e.g., 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or more than 75 percent, as compared to the seed oil level in a corresponding control plant that does not express the transgene. In some embodiments, a plant in which expression of an oil-modulating polypeptide is modulated can have decreased levels of seed oil. The seed oil level can be decreased by at least 2 percent, e.g., 2, 3, 4, 5, 10, 15, 20, 25, 30, 35, or more than 35 percent, as compared to the seed oil level in a corresponding control plant that does not express the transgene.

Plants for which modulation of levels of seed oil can be useful include, without limitation, almond, cashew, castor bean, coconut, corn, cotton, flax, hazelnut, hemp, jatropha, linseed, mustard, neem, oil palm, peanut, poppy, pumpkin, rapeseed, rice, safflower, sesame seed, soybean, sunflower, and walnut. Increases in seed oil in such plants can provide increased yields of oil extracted from the seed and increased caloric content in foodstuffs and animal feed produced from the seed. Decreases in seed oil in such plants can be useful in situations where caloric intake should be restricted.

In some embodiments, a plant in which expression of an oil-modulating polypeptide is modulated can have increased or decreased levels of oil in one or more non-seed tissues, e.g., leaf tissues, stem tissues, root or corn tissues, or fruit tissues other than seed: For example, the oil level can be increased by at least 2 percent, e.g., 2, 3, 4, 5, 6, 7, 8, 9, 10, 11, 12, 13, 14, 15, 16, 17, 18, 19, 20, 25, 30, 35, 40, 45, 50, 55, 60, 65, 70, 75 or more than 75 percent, as compared to the oil level in a corresponding control plant that does not express the transgene. In some embodiments, a plant in which expression of an oil-modulating polypeptide is modulated can have decreased levels of oil in one or more non-seed tissues. The oil level can be decreased by at least 2 percent, e.g., 2, 3, 4, 5, 10, 15, 20, 25, 30, 35, or more than 35 percent, as compared to the oil level in a corresponding control plant that does not express the transgene.

Plants for which modulation of levels of oil in non-seed tissues can be useful include, without limitation, alfalfa, apple, avocado, beans, carrot, cherry, coconut, coffee, grapefruit, lemon, lettuce, oat, olive, onion, orange, palm, peach, peanut, pear, pineapple, potato, ryegrass, sudangrass, switchgrass, and tomato. Increases in non-seed oil in such plants can provide increased oil and caloric content in edible plants, including animal forage.

In some embodiments, a plant in which expression of an oil-modulating polypeptide having an amino acid sequence corresponding to SEQ ID NO:367, SEQ ID NO:151, SEQ ID NO:162, or SEQ ID NO:148 is modulated can have increased levels of seed protein accompanying increased levels of seed oil. The protein level can be increased by at least 2 percent, e.g., 3, 4, 5, 6, 8, 10, 15, 20, 25, 30, 35, or 40 percent, as compared to the protein level in a corresponding control plant that does not express the transgene.

In some embodiments, a plant in which expression of an oil-modulating polypeptide having an amino acid sequence corresponding to SEQ ID NO:148 is modulated can have increased levels of seed protein accompanying decreased levels of seed oil. The protein level can be increased by at least 2 percent, e.g., 3, 4, 5, 6, 8, 10, 15, 20, 25, 30, 35, or 40 percent, as compared to the protein level in a corresponding control plant that does not express the transgene.

In some embodiments, a plant in which expression of an oil-modulating polypeptide having an amino acid sequence corresponding to SEQ ID NO:82 or SEQ ID NO:87 is modulated can have decreased levels of seed protein accompanying increased levels of seed oil. The protein level can be decreased by at least 2 percent, e.g., 2, 3, 4, 5, 10, 15, 20, 25, 30, 35, or more than 35 percent, as compared to the protein level in a corresponding control plant that does not express the transgene.

In some embodiments, a plant in which expression of an oil-modulating polypeptide having an amino acid sequence corresponding to SEQ ID NO:148 is modulated can have increased levels of seed oleic acid accompanying increased levels of seed oil and protein. The oleic acid level can be increased by at least 2 percent, e.g., 2, 3, 4, 5, 10, 15, 20, 25, 30, or more than 30 percent, as compared to the oleic acid level in a corresponding control plant that does not express the transgene.

In some embodiments, a plant in which expression of an oil-modulating polypeptide having an amino acid sequence corresponding to SEQ ID NO:148 is modulated can have decreased levels of seed oleic acid accompanying increased levels of seed oil and protein. The oleic acid level can be decreased by at least 2 percent, e.g., 2, 3, 4, 5, 10, 15, 20, 25, 30, or more than 30 percent, as compared to the oleic acid level in a corresponding control plant that does not express the transgene.

In some embodiments, a plant in which expression of an oil-modulating polypeptide having an amino acid sequence corresponding to SEQ ID NO:148 is modulated can have decreased level of seed oleic acid accompanying decreased levels of seed oil and increased levels of seed protein. The oleic acid level can be decreased by at least 2 percent, e.g., 2, 3, 4, 5, 10, 15, 20, 25, 30, or more than 30 percent, as compared to the oleic acid level in a corresponding control plant that does not express the transgene.

Typically, a difference (e.g., an increase) in the amount of oil or protein in a transgenic plant or cell relative to a control plant or cell is considered statistically significant at p≦0.05 with an appropriate parametric or non-parametric statistic, e.g., Chi-square test, Student's t-test, Mann-Whitney test, or F-test. In some embodiments, a difference in the amount of oil or protein is statistically significant at p<0.01, p<0.005, or p<0.001. A statistically significant difference in, for example, the amount of oil in a transgenic plant compared to the amount in cells of a control plant indicates that (1) the recombinant nucleic acid present in the transgenic plant results in altered oil levels and/or (2) the recombinant nucleic acid warrants further study as a candidate for altering the amount of oil in a plant.

Information that the polypeptides disclosed herein can modulate oil content can be useful in breeding of crop plants. Based on the effect of disclosed polypeptides on oil content, one can search for and identify polymorphisms linked to genetic loci for such polypeptides. Polymorphisms that can be identified include simple sequence repeats (SSRs), rapid amplification of polymorphic DNA (RAPDs), amplified fragment length polymorphisms (AFLPs) and restriction fragment length polymorphisms (RFLPs).

If a polymorphism is identified, its presence and frequency in populations is analyzed to determine if it is statistically significantly correlated to an alteration in oil content. Those polymorphisms that are correlated with an alteration in oil content can be incorporated into a marker assisted breeding program to facilitate the development of lines that have a desired alteration in oil content. Typically, a polymorphism identified in such a manner is used with polymorphisms at other loci that are also correlated with a desired alteration in oil content.

### Articles of Manufacture

Transgenic plants provided herein have particular uses in the agricultural and nutritional industries. For example, transgenic plants described herein can be used to make food products and animal feed. Suitable plants with which to make such products include almond, avocado, cashew, coconut, corn, flax, olive, peanut, soybean, sunflower, and walnut. Such products are useful to provide increased or decreased oil and caloric content in the diet.

Transgenic plants provided herein can also be used to make vegetable oil. Vegetable oils can be chemically extracted from transgenic plants using a solvent, such as hexane. In some cases, olive, coconut and palm oils can be produced by mechanical extraction, such as expeller-pressed extraction. Oil presses, such as the screw press and the ram press, can also be used. Suitable plants from which to make oil include almond, apricot, avocado, canola, cashew, castor bean, coconut, corn, cotton, flax, grape, hazelnut, hemp, mustard, neem, olive, palm, peanut, poppy, pumpkin, rapeseed, rice, safflower, sesame, soybean, sunflower, and walnut. Such oils can be used for frying, baking, and spray coating applications. Vegetable oils also can be used to make margarine, processed foods, oleochemicals, and essential oils. Vegetable oils are used in the electrical industry as insulators. Vegetable oils are also used as lubricants. Vegetable oil derivatives can be used in the manufacture of polymers.

Vegetable oil from transgenic plants provided herein can also be used as fuel. For example, vegetable oil can be used as fuel in a vehicle that heats the oil before it enters the fuel system. Heating vegetable oil to 150° F. reduces the viscosity of the oil sufficiently for use in diesel engines, such as Mercedes-Benz® diesel engines. The viscosity of the oil can also be reduced before it enters the tank so that neither the engine nor the vehicle needs modification. Methods of reducing oil viscosity include: transesterification, pyrolysis, micro emulsion, blending and thermal depolymerization. The transesterification refining process creates esters from vegetable oil by using an alcohol in the presence of a catalyst. This reaction takes a triglyceride molecule, or a complex fatty acid, neutralizes the free fatty acids and removes the glycerin, thereby creating an alcohol ester. One method of transesterification mixes methanol with sodium hydroxide and then aggressively mixes the resulting methoxide with vegetable oil, which results in a methyl ester. Ester-based oxygenated fuel made from vegetable oil is known as biodiesel. Biodiesel can be used as a pure fuel or blended with petroleum in any percentage. B5 biodiesel, for example, is a blend of 5% biodiesel and 95% petroleum diesel. B20 biodiesel, including BioWillie® diesel fuel, is produced by blending 20% biodiesel and 80% petroleum diesel.

Use of biodiesel is beneficial for the environment because it is associated with reduced emissions compared to the use of petroleum diesel. In addition, biodiesel is a biodegradable, nontoxic fuel that is made from renewable materials. Plants that can be used as sources of oil for biodiesel production include canola, cotton, flax, jatropha, oil palm, safflower, soybean, and sunflower.

Seeds of transgenic plants described herein can be conditioned and bagged in packaging material by means known in the art to form an article of manufacture. Packaging material such as paper and cloth are well known in the art. A package of seed can have a label e.g., a tag or label secured to the packaging material, a label printed on the packaging material, or a label inserted within the package.

The invention will be further described in the following examples, which do not limit the scope of the invention described in the claims.

### EXAMPLES

### Example 1

### Transgenic Plants

The following symbols are used in the Examples: T₁: first generation transformant; T₂: second generation, progeny of self-pollinated T₁ plants; T₃: third generation, progeny of self-pollinated T₂ plants; T₄: fourth generation, progeny of self-pollinated T₃ plants. Independent transformations are referred to as events.

The following is a list of nucleic acids that were isolated from Arabidopsis thaliana plants. SEQ ID NO:366 is a DNA clone that is predicted to encode a 294 amino acid polypeptide (SEQ ID NO:367). Ceres CDNA ID no. 12703936 (SEQ ID NO:150) is a genomic DNA clone that is predicted to encode a 221 amino acid polypeptide (genomic locus At5g12230; SEQ ID NO:151). Ceres CDNA ID no. 23649975 (SEQ ID NO:147) is a genomic DNA clone that is predicted to encode a 190 amino acid ankyrin repeat family polypeptide (genomic locus At2g26210; SEQ ID NO:148). Ceres CDNA ID no. 12706677 (SEQ ID NO:161) is a genomic DNA clone that is predicted to encode a 176 amino acid glycosyltransferase polypeptide (genomic locus At4g16710; SEQ ID NO:162). Ceres CLONE ID no. 5344 (SEQ ID NO:86) is a cDNA clone that is predicted to encode a 332 amino acid polygalacturonase inhibiting protein-1 polypeptide (genomic locus At5g06860; SEQ ID NO:87). Ceres CLONE ID no. 2721 (SEQ ID NO:202) is a DNA clone that is predicted to encode a 131 amino acid polypeptide (SEQ ID NO:203). Ceres CLONE ID no. 37493 (SEQ ID NO:244) is a DNA clone that is predicted to encode a 386 amino acid methyltransferase polypeptide (SEQ ID NO:245). Ceres CLONE ID no. 36334 (SEQ ID NO:228) is a DNA clone that is predicted to encode a 472 amino acid cytochrome P450 polypeptide (SEQ ID NO:229). Ceres CLONE ID no. 30018 (SEQ ID NO:215) is a DNA clone that is predicted to encode a 72 amino acid ubiquinol-cytochrome C reductase, UQCRX/QCR9 like polypeptide (SEQ ID NO:216). Ceres ANNOT ID no. 542494 (SEQ ID NO:361) is a DNA clone that is predicted to encode a 142 amino acid polypeptide (SEQ ID NO:175). Ceres ANNOT ID no. 841273 (SEQ ID NO:363) is a DNA clone that is predicted to encode a 262 amino acid endonuclease/exonuclease/phosphatase family polypeptide (SEQ ID NO:201). Ceres ANNOT ID no. 564261 (SEQ ID NO:364) is a DNA clone that is predicted to encode a 249 amino acid polypeptide containing a DnaJ domain (SEQ ID NO:190). Ceres ANNOT ID no. 565548 (SEQ ID NO:365) is a DNA clone that is predicted to encode a 245 amino acid Rho termination factor polypeptide (SEQ ID NO:198). Ceres ANNOT ID no. 549258 (SEQ ID NO:362) is a DNA clone that is predicted to encode a 256 amino acid acetyltransferase polypeptide (SEQ ID NO:185).

The following is a list of nucleic acids that were isolated from Glycine max plants. Ceres CLONE ID no. 590462 (SEQ ID NO:79) is a cDNA clone that is predicted to encode a 75 amino acid polypeptide (SEQ ID NO:80). Ceres CLONE ID no. 625035 (SEQ ID NO:81) is a cDNA clone that is predicted to encode a 240 amino acid AP2/EREBP transcription factor polypeptide (SEQ ID NO:82).

Each isolated nucleic acid described above was cloned into a Ti plasmid vector, CRS 338, containing a phosphinothricin acetyltransferase gene which confers Finale™ resistance to transformed plants. Constructs were made using CRS 338 that contained SEQ ID NO:366, Ceres CDNA ID no. 12703936, Ceres CDNA ID no. 23649975, Ceres CDNA ID no. 12706677, Ceres CLONE ID no. 5344, Ceres CLONE ID no. 2721, Ceres CLONE ID no. 37493, Ceres CLONE ID no. 36334, Ceres CLONE ID no. 30018, Ceres ANNOT ID no. 542494, Ceres ANNOT ID no. 841273, Ceres ANNOT ID no. 564261, Ceres ANNOT ID no. 565548, Ceres ANNOT ID no. 549258, Ceres CLONE ID no. 590462, or Ceres CLONE ID no. 625035, each operably linked to a CaMV 35S promoter. Wild-type Arabidopsis thaliana ecotype Wassilewskija (Ws) plants were transformed separately with each construct. The transformations were performed essentially as described in Bechtold et al., C.R. Acad. Sci. Paris, 316:1194-1199 (1993).

Transgenic Arabidopsis lines containing SEQ ID NO:366, Ceres CDNA ID no. 12703936, Ceres CDNA ID no. 23649975, Ceres CDNA ID no. 12706677, Ceres CLONE ID no. 5344, Ceres CLONE ID no. 2721, Ceres CLONE ID no. 37493, Ceres CLONE ID no. 36334, Ceres CLONE ID no. 30018, Ceres ANNOT ID no. 542494, Ceres ANNOT ID no. 841273, Ceres ANNOT ID no. 564261, Ceres ANNOT ID no. 565548, Ceres ANNOT ID no. 549258, Ceres CLONE ID no. 590462, or Ceres CLONE ID no. 625035 were designated ME11833, ME11278, ME11273, ME11822, ME05421, ME03392, ME03531, ME06302, ME06741, ME09515, ME11271, ME11827, ME11836, ME11837, ME03169, or ME03180, respectively. The presence of each vector containing a DNA clone described above in the respective transgenic Arabidopsis line transformed with the vector was confirmed by Finale™ resistance, polymerase chain reaction (PCR) amplification from green leaf tissue extract, and/or sequencing of PCR products. As controls, wild-type Arabidopsis ecotype Ws plants were transformed with the empty vector CRS 338.

### Example 2

### Analysis of Oil Content in Transgenic Arabidopsis Seeds

An analytical method based on Fourier transform near-infrared (FT-NIR) spectroscopy was developed, validated, and used to perform a high-throughput screen of transgenic seed lines for alterations in seed oil content. To calibrate the FT-NIR spectroscopy method, a sub-population of transgenic seed lines was randomly selected and analyzed for oil content using a direct primary method. Fatty acid methyl ester (FAME) analysis by gas chromatography-mass spectroscopy (GC-MS) was used as the direct primary method to determine the total fatty acid content for each seed line and produce the FT-NIR spectroscopy calibration curves for oil.

To analyze seed oil content using GC-MS, seed tissue was homogenized in liquid nitrogen using a mortar and pestle to create a powder. The tissue was weighed, and 5.0±0.25 mg were transferred into a 2 mL Eppendorf tube. The exact weight of each sample was recorded. One mL of 2.5% H₂SO₄ (v/v in methanol) and 20 μL of undecanoic acid internal standard (1 mg/mL in hexane) were added to the weighed seed tissue. The tubes were incubated for two hours at 90° C. in a pre-equilibrated heating block. The samples were removed from the heating block and allowed to cool to room temperature. The contents of each Eppendorf tube were poured into a 15 mL polypropylene conical tube, and 1.5 mL of a 0.9% NaCl solution and 0.75 mL of hexane were added to each tube. The tubes were vortexed for 30 seconds and incubated at room temperature for 15 minutes. The samples were then centrifuged at 4,000 rpm for 5 minutes using a bench top centrifuge. If emulsions remained, then the centrifugation step was repeated until they were dissipated. One hundred μL of the hexane (top) layer was pipetted into a 1.5 mL autosampler vial with minimum volume insert. The samples were stored no longer than 1 week at −80° C. until they were analyzed.

Samples were analyzed using a Shimadzu QP-2010 GC-MS (Shimadzu Scientific Instruments, Columbia, Md.). The first and last sample of each batch consisted of a blank (hexane). Every fifth sample in the batch also consisted of a blank. Prior to sample analysis, a 7-point calibration curve was generated using the Supelco 37 component FAME mix (0.00004 mg/mL to 0.2 mg/mL). The injection volume was 1 μL.

The GC parameters were as follows: column oven temperature: 70° C., inject temperature: 230° C., inject mode: split, flow control mode: linear velocity, column flow: 1.0 mL/min, pressure: 53.5 mL/min, total flow: 29.0 mL/min, purge flow: 3.0 mL/min, split ratio: 25.0. The temperature gradient was as follows: 70° C. for 5 minutes, increasing to 350° C. at a rate of 5 degrees per minute, and then held at 350° C. for 1 minute. The MS parameters were as follows: ion source temperature: 200° C., interface temperature: 240° C., solvent cut time: 2 minutes, detector gain mode: relative, detector gain: 0.6 kV, threshold: 1000, group: 1, start time: 3 minutes, end time: 62 minutes, ACQ mode: scan, interval: 0.5 second, scan speed: 666, start M/z: 40, end M/z: 350. The instrument was tuned each time the column was cut or a new column was used.

The data were analyzed using the Shimadzu GC-MS Solutions software. Peak areas were integrated and exported to an Excel spreadsheet. Fatty acid peak areas were normalized to the internal standard, the amount of tissue weighed, and the slope of the corresponding calibration curve generated using the FAME mixture. Peak areas were also multiplied by the volume of hexane (0.75 mL) used to extract the fatty acids.

The same seed lines that were analyzed using GC-MS were also analyzed by FT-NIR spectroscopy, and the oil values determined by the GC-MS primary method were entered into the FT-NIR chemometrics software (Bruker Optics, Billerica, Mass.) to create a calibration curve for oil content. The actual oil content of each seed line analyzed using GC-MS was plotted on the x-axis of the calibration curve. The y-axis of the calibration curve represented the predicted values based on the best-fit line. Data points were continually added to the calibration curve data set.

T₂ seed from each transgenic plant line was analyzed by FT-NIR spectroscopy. Sarstedt tubes containing seeds were placed directly on the lamp, and spectra were acquired through the bottom of the tube. The spectra were analyzed to determine seed oil content using the FT-NIR chemometrics software (Bruker Optics) and the oil calibration curve. Results for experimental samples were compared to population means and standard deviations calculated for transgenic seed lines that were planted within 30 days of the lines being analyzed and grown under the same conditions. Typically, results from three to four events of each of 400 to 1600 different transgenic lines were used to calculate a population mean. Each data point was assigned a z-score (z=(x−mean)/std), and a p-value was calculated for the z-score.

Transgenic seed lines with oil levels in T₂ seed that differed by more than two standard deviations from the population mean were selected for evaluation of oil levels in the T₃ generation. All events of selected lines were planted in individual pots. The pots were arranged randomly in flats along with pots containing matched control plants in order to minimize microenvironment effects. Matched control plants contained an empty version of the vector used to generate the transgenic seed lines. T₃ seed from up to five plants from each event was collected and analyzed individually using FT-NIR spectroscopy. Data from replicate samples were averaged and compared to controls using the Student's t-test.

### Example 3

### Analysis of Protein Content in Transgenic Arabidopsis Seeds

An analytical method based on Fourier transform near-infrared (FT-NIR) spectroscopy was developed, validated, and used to perform a high-throughput screen of transgenic seed lines for alterations in seed protein content. To calibrate the FT-NIR spectroscopy method, total nitrogen elemental analysis was used as a primary method to analyze a sub-population of randomly selected transgenic seed lines. The overall percentage of nitrogen in each sample was determined. Percent nitrogen values were multiplied by a conversion factor to obtain percent total protein values. A conversion factor of 5.30 was selected based on data for cotton, sunflower, safflower, and sesame seed (Rhee, K. C., Determination of Total Nitrogen In Handbook of Food Analytical Chemistry—Water, Proteins, Enzymes, Lipids, and Carbohydrates (R. Wrolstad et al., ed.), John Wiley and Sons, Inc., p. 105, (2005)). The same seed lines were then analyzed by FT-NIR spectroscopy, and the protein values calculated via the primary method were entered into the FT-NIR chemometrics software (Bruker Optics, Billerica, Mass.) to create a calibration curve for analysis of seed protein content by FT-NIR spectroscopy.

Elemental analysis was performed using a FlashEA 112 NC Analyzer (Thermo Finnigan, San Jose, Calif.). To analyze total nitrogen content, 2.00±0.15 mg of dried transgenic Arabidopsis seed was weighed into a tared tin cup. The tin cup with the seed was weighed, crushed, folded in half, and placed into an autosampler slot on the FlashEA 1112 NC Analyzer (Thermo Finnigan). Matched controls were prepared in a manner identical to the experimental samples and spaced evenly throughout the batch. The first three samples in every batch were a blank (empty tin cup), a bypass, (approximately 5 mg of aspartic acid), and a standard (5.00±0.15 mg aspartic acid), respectively. Blanks were entered between every 15 experimental samples. Each sample was analyzed in triplicate.

The FlashEA 1112 NC Analyzer (Thermo Finnigan) instrument parameters were as follows: left furnace 900° C., right furnace 840° C., oven 50° C., gas flow carrier 130 mL/min., and gas flow reference 100 mL/min. The data parameter LLOD was 0.25 mg for the standard and different for other materials. The data parameter LLOQ was 3.0 mg for the standard, 1.0 mg for seed tissue, and different for other materials.

Quantification was performed using the Eager 300 software (Thermo Finnigan). Replicate percent nitrogen measurements were averaged and multiplied by a conversion factor of 5.30 to obtain percent total protein values. For results to be considered valid, the standard deviation between replicate samples was required to be less than 10%. The percent nitrogen of the aspartic acid standard was required to be within ±1.0% of the theoretical value. For a run to be declared valid, the weight of the aspartic acid (standard) was required to be between 4.85 and 5.15 mg, and the blank(s) were required to have no recorded nitrogen content.

The same seed lines that were analyzed for elemental nitrogen content were also analyzed by FT-NIR spectroscopy, and the percent total protein values determined by elemental analysis were entered into the FT-NIR chemometrics software (Bruker Optics, Billerica, Mass.) to create a calibration curve for protein content. The protein content of each seed line based on total nitrogen elemental analysis was plotted on the x-axis of the calibration curve. The y-axis of the calibration curve represented the predicted values based on the best-fit line. Data points were continually added to the calibration curve data set.

T₂ seed from each transgenic plant line was analyzed by FT-NIR spectroscopy. Sarstedt tubes containing seeds were placed directly on the lamp, and spectra were acquired through the bottom of the tube. The spectra were analyzed to determine seed protein content using the FT-NIR chemometrics software (Bruker Optics) and the protein calibration curve. Results for experimental samples were compared to population means and standard deviations calculated for transgenic seed lines that were planted within 30 days of the lines being analyzed and grown under the same conditions. Typically, results from three to four events of each of 400 to 1600 different transgenic lines were used to calculate a population mean. Each data point was assigned a z-score (z=(x−mean)/std), and a p-value was calculated for the z-score.

Transgenic seed lines with oil levels in T₂ seed that differed by more than two standard deviations from the population mean were also analyzed to determine protein levels in the T₃ generation. Events of selected lines were planted in individual pots. The pots were arranged randomly in flats along with pots containing matched control plants in order to minimize microenvironment effects. Matched control plants contained an empty version of the vector used to generate the transgenic seed lines. T₃ seed from up to five plants from each event was collected and analyzed individually using FT-NIR spectroscopy. Data from replicate samples were averaged and compared to controls using the Student's t-test.

### Example 4

### Results for ME11833 Events

T₂ and T₃ seed from five events of ME1833 containing SEQ ID NO:366 was analyzed for oil content using FT-NIR spectroscopy as described in Example 2.

The oil content in T₂ seed from three events of ME11833 was significantly increased compared to the mean oil content in seed from transgenic Arabidopsis lines planted within 30 days of ME11833. As presented in Table 1, the oil content was increased to 124% in seed from event −01 and to 127% in seed from events −03 and −05 compared to the population mean.

TABLE 1
Oil content (% control) in T₂ and T₃ seed from ME11833
events containing SEQ ID NO: 366

| | Event - | Event - | Event - | Event - | Event - | |
|---|---|---|---|---|---|---|
| | 01 | 02 | 03 | 04 | 05 | Control |
| Oil content | 124 | 121 | 127 | 114 | 127 | 100 ± 0* |
| (% control) | | | | | | |
| in T₂ seed | | | | | | |
| p-value | 0.03 | 0.06 | 0.01 | 0.17 | 0.01 | N/A |
| Oil content | 106 ± 1 | 103 ± 1 | 102 | 103 ± 2 | 104 ± 1 | 100 ± 0 |
| (% control) | | | | | | |
| in T₃ seed | | | | | | |
| p-value | 0.03 | 0.01 | 0.66 | 0.29 | <0.01 | N/A |
| No. of | 3 | 5 | 1 | 4 | 3 | 15 |
| T₂ plants | | | | | | |

*Population mean of the oil content of seed from transgenic lines planted within 30 days of ME11833. Variation is presented as the standard error of the mean.

The oil content in T₃ seed from three events of ME11833 was significantly increased compared to the oil content of corresponding control seed. As presented in Table 1, the oil content was increased to 106%, 103%, and 104% in seed from events −01, −02, and −05, respectively, compared to the oil content in control seed.

T₂ and T₃ seed from five events of ME11833 containing SEQ ID NO:366 was also analyzed for protein content using FT-NIR spectroscopy as described in Example 3.

The protein content in T₂ seed from ME11833 events was not observed to differ significantly from the mean protein content in seed from transgenic Arabidopsis lines planted within 30 days of ME11833 (Table 2).

TABLE 2
Protein content (% control) in T₂ and T₃ seed from ME11833
events containing SEQ ID NO: 366

| | Event - | Event - | Event - | Event - | Event - | |
|---|---|---|---|---|---|---|
| | 01 | 02 | 03 | 04 | 05 | Control |
| Protein | 81 | 91 | 84 | 85 | 87 | 100 ± 0* |
| content | | | | | | |
| (% control) | | | | | | |
| in T₂ seed | | | | | | |
| p-value | 0.08 | 0.25 | 0.12 | 0.14 | 0.18 | N/A |
| Protein | 109 ± 2 | 111 ± 2 | 105 | 110 ± 1 | 114 ± 1 | 100 ± 1 |
| content | | | | | | |
| (% control) | | | | | | |
| in T₃ seed | | | | | | |
| p-value | 0.01 | <0.01 | 0.24 | <0.01 | <0.01 | N/A |
| No. of | 3 | 5 | 1 | 4 | 3 | 15 |
| T₂ plants | | | | | | |

*Population mean of the protein content in seed from transgenic lines planted within 30 days of ME11833. Variation is presented as the standard error of the mean.

The protein content in T₃ seed from four events of ME11833 was significantly increased compared to the protein content in corresponding control seed. As presented in Table 2, the protein content was increased to 109%, 111%, 110%, and 114% in seed from events −01, −02, −04, and −05, respectively, compared to the protein content in control seed.

The physical appearances of T₁ ME11833 plants were similar to those of corresponding control plants. There were no observable or statistically significant differences between T₂ ME11833 and control plants in germination, onset of flowering, rosette area, fertility, and general morphology/architecture.

### Example 5

### Results for ME11278 Events

T₂ and T₃ seed from five events of ME11278 containing Ceres CDNA ID no. 12703936 was analyzed for oil content using FT-NIR spectroscopy as described in Example 2.

The oil content in T₂ seed from five events of ME11278 was significantly increased compared to the mean oil content in seed from transgenic Arabidopsis lines planted within 30 days of ME11278. As presented in Table 3, the oil content was increased to 122%, 128%, 120%, 121%, and 123% in seed from events −01, −02, −03, −04, and −05, respectively, compared to the population mean.

TABLE 3
Oil content (% control) in T₂ and T₃ seed from ME11278
events containing Ceres CDNA ID no. 12703936

| | Event - | Event - | Event - | Event - | Event - | |
|---|---|---|---|---|---|---|
| | 01 | 02 | 03 | 04 | 05 | Control |
| Oil content | 122 | 128 | 120 | 121 | 123 | 100 ± 0* |
| (% control) | | | | | | |
| in T₂ seed | | | | | | |
| p-value | 0.03 | <0.01 | 0.05 | 0.03 | 0.02 | N/A |
| Oil content | 103 ± 0 | 105 ± 0 | 102 ± 0 | 101 ± 2 | 105 ± 1 | 100 ± 0 |
| (% control) | | | | | | |
| in T₃ seed | | | | | | |
| p-value | <0.01 | <0.01 | 0.01 | 0.76 | 0.04 | N/A |
| No. of | 5 | 5 | 5 | 2 | 3 | 15 |
| T₂ plants | | | | | | |

*Population mean of the oil content in seed from transgenic lines planted within 30 days of ME11278. Variation is presented as the standard error of the mean.

The oil content in T₃ seed from four events of ME11278 was significantly increased compared to the oil content in corresponding control seed. As presented in Table 3, the oil content was increased to 103%, 105%, 102%, and 105% in seed from events −01, −02, −03, and −05, respectively, compared to the oil content in control seed.

T₂ and T₃ seed from five events of ME11278 containing Ceres CDNA ID no. 12703936 was also analyzed for total protein content using FT-NIR spectroscopy as described in Example 3.

The protein content in T₂ seed from ME11278 events was not observed to differ significantly from the mean protein content in seed from transgenic Arabidopsis lines planted within 30 days of ME11278 (Table 4).

TABLE 4
Protein content (% control) in T₂ and T₃ seed from ME11278
events containing Ceres CDNA ID no. 12703936

| | Event - | Event - | Event - | Event - | Event - | |
|---|---|---|---|---|---|---|
| | 01 | 02 | 03 | 04 | 05 | Control |
| Protein | 98 | 99 | 97 | 102 | 107 | 100 ± 0* |
| content | | | | | | |
| (% control) | | | | | | |
| in T₂ seed | | | | | | |
| p-value | 0.25 | 0.25 | 0.24 | 0.25 | 0.23 | N/A |
| Protein | 109 ± 1 | 115 ± 1 | 113 ± 2 | 117 ± 2 | 115 ± 5 | 100 ± 1 |
| content | | | | | | |
| (% control) | | | | | | |
| in T₃ seed | | | | | | |
| p-value | <0.01 | <0.01 | <0.01 | 0.06 | 0.11 | N/A |
| No. of | 5 | 5 | 5 | 2 | 3 | 15 |
| T₂ plants | | | | | | |

*Population mean of the protein content in seed from transgenic lines planted within 30 days of ME11278. Variation is presented as the standard error of the mean.

The protein content in T₃ seed from three events of ME11278 was significantly increased compared to the protein content in corresponding control seed. As presented in Table 4, the protein content was increased to 109%, 115%, and 113% in seed from events −01, −02, and −03, respectively, compared to the protein content in control seed.

The physical appearances of T₁ ME11278 plants were similar to those of corresponding control plants. There were no observable or statistically significant differences between T₂ ME11278 and control plants in germination, onset of flowering, rosette area, fertility, and general morphology/architecture.

### Example 6

### Results for ME11822 Events

T₂ and T₃ seed from five events of ME11822 containing Ceres CDNA ID no. 12706677 was analyzed for oil content using FT-NIR spectroscopy as described in Example 2.

The oil content in T₂ seed from four events of ME11822 was significantly increased compared to the mean oil content in seed from transgenic Arabidopsis lines planted within 30 days of ME11822. As presented in Table 5, the oil content was increased to 123% in seed from event −01 and to 122% in seed from events −02, −03, and −05 compared to the population mean.

TABLE 5
Oil content (% control) in T₂ and T₃ seed from ME11822 events
containing Ceres CDNA ID no. 12706677

| | Event - | Event - | Event - | Event - | Event - | |
|---|---|---|---|---|---|---|
| | 01 | 02 | 03 | 04 | 05 | Control |
| Oil content | 123 | 122 | 122 | 120 | 122 | 100 ± 0* |
| (% control) | | | | | | |
| in T₂ seed | | | | | | |
| p-value | 0.04 | 0.05 | 0.05 | 0.07 | 0.05 | N/A |
| Oil content | 101 ± 0 | 104 ± 1 | 105 ± 0 | 100 ± 1 | 100 ± 2 | 100 ± 0 |
| (% control) | | | | | | |
| in T₃ seed | | | | | | |
| p-value | 0.07 | <0.01 | <0.01 | 0.85 | 0.97 | N/A |
| No. of | 5 | 4 | 5 | 3 | 3 | 15 |
| T₂ plants | | | | | | |

*Population mean of the oil content in seed from transgenic lines planted within 30 days of ME11822. Variation is presented as the standard error of the mean.

The oil content in T₃ seed from two events of ME11822 was significantly increased compared to the oil content in corresponding control seed. As presented in Table 5, the oil content was increased to 104% and 105% in seed from events −02 and −03, respectively, compared to the oil content in control seed.

T₂ and T₃ seed from five events of ME11822 containing Ceres CDNA ID no. 12706677 was also analyzed for total protein content using FT-NIR spectroscopy as described in Example 3.

The protein content in T₂ seed from ME11822 events was not observed to differ significantly from the mean protein content in seed from transgenic Arabidopsis lines planted within 30 days of ME11822 (Table 6).

TABLE 6
Protein content (% control) in T₂ and T₃ seed from ME11822 events
containing Ceres CDNA ID no. 12706677

| | Event - | Event - | Event - | Event - | Event - | |
|---|---|---|---|---|---|---|
| | 01 | 02 | 03 | 04 | 05 | Control |
| Protein | 87 | 92 | 84 | 98 | 93 | 100 ± 0* |
| content | | | | | | |
| (% control) | | | | | | |
| in T₂ seed | | | | | | |
| p-value | 0.17 | 0.27 | 0.12 | 0.34 | 0.28 | N/A |
| Protein | 108 ± 1 | 110 ± 3 | 108 ± 2 | 109 ± 2 | 109 ± 2 | 100 ± 1 |
| content | | | | | | |
| (% control) | | | | | | |
| in T₃ seed | | | | | | |
| p-value | <0.01 | 0.03 | 0.01 | 0.03 | 0.01 | N/A |
| No. of | 5 | 4 | 5 | 3 | 3 | 15 |
| T₂ plants | | | | | | |

*Population mean of the protein content in seed from transgenic lines planted within 30 days of ME11822. Variation is presented as the standard error of the mean.

The protein content in T₃ seed from five events of ME11822 was significantly increased compared to the protein content in corresponding control seed. As presented in Table 6, the protein content was increased to 108% in seed from events −01 and −03, to 110% in seed from event −02, and to 109% in seed from events −04 and −05 compared to the protein content in control seed.

The physical appearances of T₁ ME11822 plants were similar to those of corresponding control plants. There were no observable or statistically significant differences between T₂ ME11822 and control plants in germination, onset of flowering, rosette area, fertility, and general morphology/architecture.

### Example 7

### Results for ME11273 Events

T₂ and T₃ seed from five events of ME11273 containing Ceres CDNA ID no. 23649975 was analyzed for oil content using FT-NIR spectroscopy as described in Example 2.

The oil content in T₂ seed from three events of ME11273 was significantly increased compared to the mean oil content in seed from transgenic Arabidopsis lines planted within 30 days of ME11273. As presented in Table 7, the oil content was increased to 129%, 120%, and 123% in seed from events −01, −02, and −05, respectively, compared to the population mean.

TABLE 7
Oil content (% control) in T₂ and T₃ seed from ME11273 events
containing Ceres CDNA ID no. 23649975

| | Event - | Event - | Event - | Event - | Event - | |
|---|---|---|---|---|---|---|
| | 01 | 02 | 03 | 04 | 05 | Control |
| Oil content | 129 | 120 | 102 | 115 | 123 | 100 ± 0* |
| (% control) | | | | | | |
| in T₂ seed | | | | | | |
| p-value | <0.01 | 0.04 | 0.47 | 0.13 | 0.02 | N/A |
| Oil content | 105 ± 0 | 105 ± 1 | 97 ± 1 | 104 ± 0 | 102 ± 1 | 100 ± 0 |
| (% control) | | | | | | |
| in T₃ seed | | | | | | |
| p-value | <0.01 | <0.01 | 0.03 | <0.01 | 0.19 | N/A |
| No. of | 3 | 5 | 5 | 3 | 3 | 15 |
| T₂ plants | | | | | | |

*Population mean of the oil content in seed from transgenic lines planted within 30 days of ME11273. Variation is presented as the standard error of the mean.

The oil content in T₃ seed from three events of ME11273 was significantly increased compared to the oil content in corresponding control seed. As presented in Table 7, the oil content was increased to 105% in seed from events −01 and −02 and to 104% in seed from event −04 compared to the oil content in control seed. The oil content in T₃ seed from one event of ME11273 was significantly decreased compared to the oil content in corresponding control seed. As presented in Table 7, the oil content was decreased to 97% in seed from event −03 compared to the oil content in control seed.

T₂ and T₃ seed from four events of ME11273 containing Ceres CDNA ID no. 23649975 was also analyzed for oleic acid content using GC-MS as described in Example 2. For each event, the area under the peak in the chromatogram corresponding to oleic acid was normalized to the internal standard, and the normalized peak areas were compared to those from empty vector transgenic controls processed and analyzed in a similar manner.

The oleic acid content in T₂ seed from two events of ME11273 was significantly increased compared to the mean oleic acid content in seed from empty vector transgenic Arabidopsis controls. As presented in Table 8, the oleic acid content was increased to 131% and 122% in seed from events −01 and −02, respectively, compared to controls. The oleic acid content in T₂ seed from two events of ME11273 was significantly decreased compared to the mean oleic acid content in seed from empty vector transgenic Arabidopsis controls. As presented in Table 8, the oleic acid content was decreased to 60% and 68% in seed from events −03 and −04, respectively, compared to controls.

TABLE 8
Oleic acid content (% control) in T₂ and T₃ seed from ME11273 events
containing Ceres CDNA ID no. 23649975

| | Event -01 | Event -02 | Event -03 | Event -04 | Control |
|---|---|---|---|---|---|
| Oleic acid | 131 ± 2 | 122 ± 1 | 60 ± 1 | 68 ± 1 | 100 ± 10 |
| content | | | | | |
| (% control) | | | | | |
| in T₂ seed | | | | | |
| p-value | 0.01 | <0.01 | <0.01 | <0.01 | N/A |
| Oleic acid | 130 ± 3 | 123 ± 4 | 101 ± 2 | 109 ± 3 | 100 ± 9 |
| content | | | | | |
| (% control) | | | | | |
| in T₃ seed | | | | | |
| p-value | <0.01 | 0.01 | 0.84 | 0.13 | N/A |
| No. of | 5 | 5 | 5 | 5 | 15 |
| T₂ plants | | | | | |

Variation is presented as the standard error of the mean.

The oleic acid content in T₃ seed from two events of ME11273 was significantly increased compared to the oleic acid content in corresponding control seed. As presented in Table 8, the oleic acid content was increased to 130% and 123% in seed from events −01 and −02, respectively, compared to the oleic acid content in corresponding control seed.

T₂ and T₃ seed from five events of ME11273 containing Ceres CDNA ID no. 23649975 was also analyzed for total protein content using FT-NIR spectroscopy as described in Example 3.

The protein content in T₂ seed from ME11273 events was not observed to differ significantly from the mean protein content in seed from transgenic Arabidopsis lines planted within 30 days of ME11273 (Table 9).

TABLE 9
Protein content (% control) in T₂ and T₃ seed from ME11273 events
containing Ceres CDNA ID no. 23649975

| | Event - | Event - | Event - | Event - | Event - | |
|---|---|---|---|---|---|---|
| | 01 | 02 | 03 | 04 | 05 | Control |
| Protein | 100 | 96 | 95 | 83 | 92 | 100 ± 0* |
| content | | | | | | |
| (% control) | | | | | | |
| in T₂ seed | | | | | | |
| p-value | 0.25 | 0.24 | 0.24 | 0.17 | 0.23 | N/A |
| Protein | 113 ± 0 | 104 ± 1 | 108 ± 1 | 106 ± 1 | 107 ± 2 | 100 ± 1 |
| content | | | | | | |
| (% control) | | | | | | |
| in T₃ seed | | | | | | |
| p-value | <0.01 | 0.01 | <0.01 | <0.01 | 0.04 | N/A |
| No. of | 3 | 5 | 5 | 3 | 3 | 15 |
| T₂ plants | | | | | | |

*Population mean of the protein content in seed from transgenic lines planted within 30 days of ME11273. Variation is presented as the standard error of the mean.

The protein content in T₃ seed from five events of ME11273 was significantly increased compared to the protein content in corresponding control seed. As presented in Table 9, the protein content was increased to 113%, 104%, 108%, 106%, and 107% in seed from events −01, −02, −03, −04, and −05, respectively, compared to the protein content in control seed.

The physical appearances of T₁ ME11273 plants were similar to those of corresponding control plants. There were no observable or statistically significant differences between T₂ ME11273 and control plants in germination, onset of flowering, rosette area, fertility, and general morphology/architecture.

### Example 8

### Results for ME03169 Events

T₂ and T₃ seed from four events and five events, respectively, of ME03169 containing Ceres CLONE ID no. 590462 was analyzed for oil content using FT-NIR spectroscopy as described in Example 2.

The oil content in T₂ seed from three events of ME03169 was significantly increased compared to the mean oil content in seed from transgenic Arabidopsis lines planted within 30 days of ME03169. As presented in Table 10, the oil content was increased to 125%, 118%, and 115% in seed from events −06, −07, and −09, respectively, compared to the population mean.

TABLE 10
Oil content (% control) in T₂ and T₃ seed from ME03169 events
containing Ceres CLONE ID no. 590462

| | Event - | Event - | Event - | Event - | Event - | |
|---|---|---|---|---|---|---|
| | 05 | 06 | 07 | 08 | 09 | Control |
| Oil content | 108 | 125 | 118 | No data | 115 | 100 ± 0* |
| (% control) | | | | | | |
| in T₂ seed | | | | | | |
| p-value | 0.25 | <0.01 | <0.01 | No data | 0.02 | N/A |
| Oil content | 103 ± 1 | 105 ± 0 | 103 ± 1 | 102 ± 2 | 105 ± 0 | 100 ± 1 |
| (% control) | | | | | | |
| in T₃ seed | | | | | | |
| p-value | 0.09 | <0.01 | 0.02 | 0.33 | <0.01 | N/A |
| No. of | 3 | 5 | 3 | 3 | 5 | 29 |
| T₂ plants | | | | | | |

*Population mean of the oil content in seed from transgenic lines planted within 30 days of ME03169. Variation is presented as the standard error of the mean.

The oil content in T₃ seed from three events of ME03169 was significantly increased compared to the oil content in corresponding control seed. As presented in Table 10, the oil content was increased to 105% in seed from events −06 and −09 and to 103% in seed from event −07 compared to the oil content in control seed.

T₂ and T₃ seed from four events and five events, respectively, of ME03169 containing Ceres CLONE ID no. 590462 was also analyzed for total protein content using FT-NIR spectroscopy as described in Example 3. The protein content in T₂ and T₃ seed from ME03169 events was not observed to differ significantly from the protein content in corresponding control seed (Table 11).

TABLE 11
Protein content (% control) in T₂ and T₃ seed from ME03169 events
containing Ceres CLONE ID no. 590462

| | Event - | Event - | Event - | Event - | Event - | |
|---|---|---|---|---|---|---|
| | 05 | 06 | 07 | 08 | 09 | Control |
| Protein | 94 | 105 | 109 | No data | 101 | 100 ± 0* |
| content | | | | | | |
| (% control) | | | | | | |
| in T₂ seed | | | | | | |
| p-value | 0.33 | 0.31 | 0.37 | No data | 0.21 | N/A |
| Protein | 99 ± 1 | 101 ± 2 | 100 ± 2 | 100 ± 2 | 101 ± 3 | 100 ± 1 |
| content | | | | | | |
| (% control) | | | | | | |
| in T₃ seed | | | | | | |
| p-value | 0.58 | 0.67 | 0.99 | 0.90 | 0.81 | N/A |
| No. of | 3 | 5 | 3 | 3 | 5 | 29 |
| T₂ plants | | | | | | |

*Population mean of the protein content in seed from transgenic lines planted within 30 days of ME03169. Variation is presented as the standard error of the mean.

The physical appearances of T₁ ME03169 plants were similar to those of corresponding control plants. There were no observable or statistically significant differences between T₂ ME03169 and control plants in germination, onset of flowering, rosette area, fertility, and general morphology/architecture. T₃ plants in one out of two pots from each of events −06 and −07 were observed to have curled rosette leaves and a smaller stature.

### Example 9

### Results for ME03180 Events

T₂ and T₃ seed from four events of ME03180 containing Ceres CLONE ID no. 625035 was analyzed for oil content using FT-NIR spectroscopy as described in Example 2.

The oil content in T₂ seed from two events of ME03180 was significantly increased compared to the mean oil content in seed from transgenic Arabidopsis lines planted within 30 days of ME03180. As presented in Table 12, the oil content was increased to 120% and 115% in seed from events −01 and −05, respectively, compared to the population mean.

TABLE 12
Oil content (% control) in T₂ and T₃ seed
from ME03180 events containing Ceres CLONE ID no. 625035

| | Event-01 | Event-02 | Event-03 | Event-05 | Control |
|---|---|---|---|---|---|
| Oil content | 120 | 106 | 99 | 115 | 100 ± 0* |
| (% control) | | | | | |
| in T₂ seed | | | | | |
| p-value | <0.01 | 0.38 | 0.63 | 0.02 | N/A |
| Oil content | 105 ± 1 | 100 ± 1 | 102 ± 1 | 103 ± 1 | 100 ± 1 |
| (% control) | | | | | |
| in T₃ seed | | | | | |
| p-value | <0.01 | 0.87 | 0.06 | 0.01 | N/A |
| No. of T₂ | 5 | 4 | 5 | 4 | 29 |
| plants | | | | | |

*Population mean of the oil content in seed from transgenic lines planted within 30 days of ME03180. Variation is presented as the standard error of the mean.

The oil content in T₃ seed from two events of ME03180 was significantly increased compared to the oil content in corresponding control seed. As presented in Table 12, the oil content was increased to 105% and 103% in seed from events −01 and −05, respectively, compared to the oil content in control seed.

T₂ and T₃ seed from four events of ME03180 containing Ceres CLONE ID no. 625035 was also analyzed for total protein content using FT-NIR spectroscopy as described in Example 3.

The protein content in T₂ seed from ME03180 events was not observed to differ significantly from the mean protein content in seed from transgenic Arabidopsis lines planted within 30 days of ME03180 (Table 13).

TABLE 13
Protein content (% control) in T₂ and T₃ seed
from ME03180 events containing Ceres CLONE ID no. 625035

| | Event-01 | Event-02 | Event-03 | Event-05 | Control |
|---|---|---|---|---|---|
| Protein | 111 | 97 | 99 | 106 | 100 ± 0* |
| content | | | | | |
| (% control) | | | | | |
| in T₂ seed | | | | | |
| p-value | 0.17 | 0.34 | 0.36 | 0.29 | N/A |
| Protein | 91 ± 1 | 94 ± 1 | 94 ± 1 | 99 ± 2 | 100 ± 1 |
| content | | | | | |
| (% control) | | | | | |
| in T₃ seed | | | | | |
| p-value | <0.01 | 0.03 | <0.01 | 0.59 | N/A |
| No. of | 5 | 4 | 5 | 4 | 29 |
| T₂ plants | | | | | |

*Population mean of the protein content in seed from transgenic lines planted within 30 days of ME03180. Variation is presented as the standard error of the mean.

The protein content in T₃ seed from three events of ME03180 was significantly decreased compared to the protein content in corresponding control seed. As presented in Table 13, the protein content was decreased to 91% in seed from event −01 and o 94% in seed from events −02 and −03 compared to the protein content in control seed.

The physical appearances of T₁ ME03180 plants were similar to those of corresponding control plants. There were no observable or statistically significant differences between T₂ ME03180 and control plants in germination, onset of flowering, rosette area, fertility, and general morphology/architecture.

### Example 10

### Results for ME05421 Events

T₂ and T₃ seed from four events and five events, respectively, of ME05421 containing Ceres CLONE ID no. 5344 was analyzed for oil content using FT-NIR spectroscopy as described in Example 2.

The oil content in T₂ seed from four events of ME05421 was significantly increased compared to the mean oil content in seed from transgenic Arabidopsis lines planted within 30 days of ME05421. As presented in Table 14, the oil content was increased to 114% in seed from events −01 and −05 and to 124% and 115% in seed from events −02 and −03, respectively, compared to the population mean.

TABLE 14
Oil content (% control) in T₂ and T₃ seed
from ME05421 events containing Ceres CLONE ID no. 5344

| | Event- | Event- | Event- | Event- | Event- | |
|---|---|---|---|---|---|---|
| | 01 | 02 | 03 | 04 | 05 | Control |
| Oil content | 114 | 124 | 115 | No data | 114 | 100 ± 0* |
| (% control) | | | | | | |
| in T_{2 seed} | | | | | | |
| p-value | 0.05 | <0.01 | 0.04 | No data | 0.05 | N/A |
| Oil content | 102 ± 2 | 103 ± 1 | 101 ± 2 | 102 ± 3 | 104 ± 0 | 100 ± 1 |
| (% control) | | | | | | |
| in T₃ seed | | | | | | |
| p-value | 0.32 | <0.01 | 0.61 | 0.18 | <0.01 | N/A |
| No. of | 5 | 5 | 5 | 3 | 4 | 29 |
| T₂ plants | | | | | | |

*Population mean of the oil content in seed from transgenic lines planted within 30 days of ME05421. Variation is presented as the standard error of the mean.

The oil content in T₃ seed from two events of ME05421 was significantly increased compared to the oil content in corresponding control seed. As presented in Table 14, the oil content was increased to 103% and 104% in seed from events −02 and −05, respectively, compared to the oil content in control seed.

T₂ and T₃ seed from four events and five events, respectively, of ME05421 containing Ceres CLONE ID no. 5344 was also analyzed for total protein content using FT-NIR spectroscopy as described in Example 3.

The protein content in T₂ seed from ME05421 events was not observed to differ significantly from the mean protein content in seed from transgenic Arabidopsis lines planted within 30 days of ME05421 (Table 15).

TABLE 15
Protein content (% control) in T₂ and T₃ seed
from ME05421 events containing Ceres CLONE ID no. 5344

| | Event- | Event- | Event- | Event- | Event- | |
|---|---|---|---|---|---|---|
| | 01 | 02 | 03 | 04 | 05 | Control |
| Protein | 89 | 92 | 102 | No data | 92 | 100 ± 0* |
| content | | | | | | |
| (% control) | | | | | | |
| in T₂ seed | | | | | | |
| p-value | 0.21 | 0.26 | 0.31 | No data | 0.25 | N/A |
| Protein | 86 ± 1 | 90 ± 2 | 89 ± 2 | 83 ± 1 | 83 ± 1 | 100 ± 1 |
| content | | | | | | |
| (% control) | | | | | | |
| in T₃ seed | | | | | | |
| p-value | <0.01 | <0.01 | <0.01 | <0.01 | <0.01 | N/A |
| No. of | 5 | 5 | 5 | 3 | 4 | 29 |
| T₂ plants | | | | | | |

*Population mean of the protein content in seed from transgenic lines planted within 30 days of ME05421. Variation is presented as the standard error of the mean.

The protein content in T₃ seed from five events of ME05421 was significantly decreased compared to the protein content in corresponding control seed. As presented in Table 15, the protein content was decreased to 86%, 90%, and 89% in seed from events −01, 02, and −03, respectively, and to 83% in seed from events −04 and −05 compared to the protein content in control seed.

The physical appearances of T₁ ME05421 plants were similar to those of corresponding control plants. There were no observable or statistically significant differences between T₂ ME05421 and control plants in germination, onset of flowering, rosette area, and general morphology/architecture. T₂ plants from event −05 of ME05421 had a decreased yield of seed relative to corresponding control plants.

### Example 11

### Results for ME03392 Events

T₂ and T₃ seed from five events and two events, respectively, of ME03392 containing Ceres CLONE ID no. 2721 was analyzed for oil content using FT-NIR spectroscopy as described in Example 2.

The oil content in T₂ seed from three events of ME03392 was significantly increased compared to the mean oil content in seed from transgenic Arabidopsis lines planted within 30 days of ME03392. As presented in Table 16, the oil content was increased to 123%, 115%, and 116% in seed from events −01, −02, and −04, respectively, compared to the population mean.

TABLE 16
Oil content (% control) in T₂ and T₃ seed from
ME03392 events containing Ceres CLONE ID no. 2721

| | Event- | Event- | Event- | Event- | Event- | |
|---|---|---|---|---|---|---|
| | 01 | 02 | 03 | 04 | 05 | Control |
| Oil content | 123 | 115 | 105 | 116 | 105 | 100 ± 0* |
| (% control) | | | | | | |
| in T₂ seed | | | | | | |
| p-value | <0.01 | 0.02 | 0.46 | 0.02 | 0.43 | N/A |
| Oil content | No data | 100 ± 2 | No data | 111 ± 1 | No data | 100 ± 1 |
| (% control) | | | | | | |
| in T₃ seed | | | | | | |
| p-value | No data | 0.95 | No data | <0.01 | No data | N/A |

*Population mean of the oil content in seed from transgenic lines planted within 30 days of ME03392. Variation is presented as the standard error of the mean.

The oil content in T₃ seed from one event of ME03392 was significantly increased compared to the oil content in corresponding control seed. As presented in Table 16, the oil content was increased to 111% in seed from event −04 compared to the oil content in control seed.

### Example 12

### Results for ME03531 Events

T₂ and T₃ seed from four events and two events, respectively, of ME03531 containing Ceres CLONE ID no. 37493 was analyzed for oil content using FT-NIR spectroscopy as described in Example 2.

The oil content in T₂ seed from two events of ME03531 was significantly increased compared to the mean oil content in seed from transgenic Arabidopsis lines planted within 30 days of ME03531. As presented in Table 17, the oil content was increased to 118% and 116% in seed from events −03 and −05, respectively, compared to the population mean.

TABLE 17
Oil content (% control) in T₂ and T₃ seed from
ME03531 events containing Ceres CLONE ID no. 37493

| | Event-02 | Event-03 | Event-05 | Event-08 | Control |
|---|---|---|---|---|---|
| Oil content | 112 | 118 | 116 | 104 | 100 ± 0* |
| (% control) | | | | | |
| in T₂ seed | | | | | |
| p-value | 0.09 | 0.01 | 0.02 | 0.52 | N/A |
| Oil content | No data | 114 | No data | 101 ± 2 | 100 ± 1 |
| (% control) | | | | | |
| in T₃ seed | | | | | |
| p-value | No data | <0.01 | No data | 0.35 | N/A |

*Population mean of the oil content in seed from transgenic lines planted within 30 days of ME03531. Variation is presented as the standard error of the mean.

The oil content in T₃ seed from one event of ME03531 was significantly increased compared to the oil content in corresponding control seed. As presented in Table 17, the oil content was increased to 114% in seed from event −03 compared to the oil content in control seed.

### Example 13

### Results for ME06302 Events

T₂ and T₃ seed from five events and four events, respectively, of ME06302 containing Ceres CLONE D no. 36334 was analyzed for oil content using FT-NIR spectroscopy as described in Example 2.

The oil content in T₂ seed from two events of ME06302 was significantly increased compared to the mean oil content in seed from transgenic Arabidopsis lines planted within 30 days of ME06302. As presented in Table 18, the oil content was increased to 114% and 117% in seed from events −02 and −07, respectively, compared to the population mean.

TABLE 18
Oil content (% control) in T₂ and T₃ seed from ME06302 events containing Ceres
CLONE ID no. 36334

| | Event- | Event- | Event- | Event- | Event- | Event- | |
|---|---|---|---|---|---|---|---|
| | 01 | 02 | 04 | 07 | 09 | 10 | Control |
| Oil content (% control) | No data | 114 | 105 | 117 | 111 | 103 | 100 ± 0* |
| in T₂ seed | | | | | | | |
| p-value | No data | 0.05 | 0.48 | 0.01 | 0.14 | 0.56 | N/A |
| Oil content (% control) | 105 | No data | 101 ± 2 | 107 ± 1 | No data | 99 ± 1 | 100 ± 1 |
| in T₃ seed | | | | | | | |
| p-value | 0.22 | No data | 0.64 | <0.01 | No data | 0.19 | N/A |

* Population mean of the oil content in seed from transgenic lines planted within 30 days of ME06302. Variation is presented as the standard error of the mean.

The oil content in T₃ seed from one event of ME06302 was significantly increased compared to the oil content in corresponding control seed. As presented in Table 18, the oil content was increased to 107% in seed from event −07 compared to the oil content in control seed.

### Example 14

### Results for ME11271 Events

T₂ and T₃ seed from five events of ME11271 containing Ceres ANNOT ID no. 841273 was analyzed for oil content using FT-NIR spectroscopy as described in Example 2.

The oil content in T₂ seed from four events of ME11271 was significantly increased compared to the mean oil content in seed from transgenic Arabidopsis lines planted within 30 days of ME11271. As presented in Table 19, the oil content was increased to 122% in seed from events −02, −04, and −05 and to 121% in seed from event −03 compared to the population mean.

TABLE 19
Oil content (% control) in T₂ and T₃ seed from
ME11271 events containing Ceres ANNOT ID no. 841273

| | Event- | Event- | Event- | Event- | Event- | |
|---|---|---|---|---|---|---|
| | 01 | 02 | 03 | 04 | 05 | Control |
| Oil content | 115 | 122 | 121 | 122 | 122 | 100 ± 0* |
| (% control) | | | | | | |
| in T₂ seed | | | | | | |
| p-value | 0.13 | 0.03 | 0.04 | 0.03 | 0.03 | N/A |
| Oil content | 100 ± 1 | 102 ± 2 | 101 ± 1 | 104 ± 1 | 99 ± 3 | 100 ± 2 |
| (% control) | | | | | | |
| in T₃ seed | | | | | | |
| p-value | 0.83 | 0.11 | 0.23 | <0.01 | 0.65 | N/A |

*Population mean of the oil content in seed from transgenic lines planted within 30 days of ME11271. Variation is presented as the standard error of the mean.

The oil content in T₃ seed from one event of ME11271 was significantly increased compared to the oil content in corresponding control seed. As presented in Table 19, the oil content was increased to 104% in seed from event −04 compared to the oil content in control seed.

### Example 15

### Results for ME11827 Events

T₂ and T₃ seed from five events of ME11827 containing Ceres ANNOT ID no. 564261 was analyzed for oil content using FT-NIR spectroscopy as described in Example 2.

The oil content in T₂ seed from three events of ME11827 was significantly increased compared to the mean oil content in seed from transgenic Arabidopsis lines planted within 30 days of ME11827. As presented in Table 20, the oil content was increased to 122% in seed from event −01 and to 123% in seed from events −04 and −05 compared to the population mean.

TABLE 20
Oil content (% control) in T₂ and T₃ seed from
ME11827 events containing Ceres ANNOT ID no. 564261

| | Event- | Event- | Event- | Event- | Event- | |
|---|---|---|---|---|---|---|
| | 01 | 02 | 03 | 04 | 05 | Control |
| Oil content | 122 | No data | 119 | 123 | 123 | 100 ± 0* |
| (% control) | | | | | | |
| in T₂ seed | | | | | | |
| p-value | 0.05 | No data | 0.08 | 0.04 | 0.03 | N/A |
| Oil content | 102 ± 2 | 101 ± 1 | 106 ± 2 | 104 ± 2 | 100 ± 2 | 100 ± 2 |
| (% control) | | | | | | |
| in T₃ seed | | | | | | |
| p-value | 0.12 | 0.27 | <0.01 | 0.01 | 0.72 | N/A |

*Population mean of the oil content in seed from transgenic lines planted within 30 days of ME11827. Variation is presented as the standard error of the mean.

The oil content in T₃ seed from two events of ME11827 was significantly increased compared to the oil content in corresponding control seed. As presented in Table 20, the oil content was increased to 106% and 104% in seed from events −03 and −04, respectively, compared to the oil content in control seed.

### Example 16

### Results for ME11836 Events

T₂ and T₃ seed from five events of ME11836 containing Ceres ANNOT ID no. 565548 was analyzed for oil content using FT-NIR spectroscopy as described in Example 2.

The oil content in T₂ seed from three events of ME11836 was significantly increased compared to the mean oil content in seed from transgenic Arabidopsis lines planted within 30 days of ME11836. As presented in Table 21, the oil content was increased to 125%, 126%, and 127% in seed from events −02, −03, and −04, respectively, compared to the population mean.

TABLE 21
Oil content (% control) in T₂ and T₃ seed from
ME11836 events containing Ceres ANNOT ID no. 565548

| | Event- | Event- | Event- | Event- | Event- | |
|---|---|---|---|---|---|---|
| | 01 | 02 | 03 | 04 | 05 | Control |
| Oil content | 117 | 125 | 126 | 127 | 116 | 100 ± 0* |
| (% control) | | | | | | |
| in T₂ seed | | | | | | |
| p-value | 0.11 | 0.03 | 0.02 | 0.01 | 0.13 | N/A |
| Oil content | 101 ± 2 | 99 ± 2 | No data | 106 ± 1 | 105 ± 1 | 100 ± 2 |
| (% control) | | | | | | |
| in T₃ seed | | | | | | |
| p-value | 0.68 | 0.24 | No data | <0.01 | <0.01 | N/A |

*Population mean of the oil content in seed from transgenic lines planted within 30 days of ME11836. Variation is presented as the standard error of the mean.

The oil content in T₃ seed from two events of ME11836 was significantly increased compared to the oil content in corresponding control seed. As presented in Table 21, the oil content was increased to 106% and 105% in seed from events −04 and −05, respectively, compared to the oil content in control seed.

### Example 17

### Results for ME11837 Events

T₂ and T₃ seed from five events of ME11837 containing Ceres ANNOT ID no. 549258 was analyzed for oil content using FT-NIR spectroscopy as described in Example 2.

The oil content in T₂ seed from three events of ME11837 was significantly increased compared to the mean oil content in seed from transgenic Arabidopsis lines planted within 30 days of ME11837. As presented in Table 22, the oil content was increased to 128%, 124%, and 127% in seed from events −01, −02, and −03, respectively, compared to the population mean.

TABLE 22
Oil content (% control) in T₂ and T₃ seed from
ME11837 events containing Ceres ANNOT ID no. 549258

| | Event- | Event- | Event- | Event- | Event- | |
|---|---|---|---|---|---|---|
| | 01 | 02 | 03 | 04 | 05 | Control |
| Oil content | 128 | 124 | 127 | 118 | 105 | 100 ± 0* |
| (% control) | | | | | | |
| in T₂ seed | | | | | | |
| p-value | 0.01 | 0.03 | 0.01 | 0.09 | 0.39 | N/A |
| Oil content | 99 ± 1 | 105 ± 2 | 99 ± 1 | No data | 101 ± 1 | 100 ± 2 |
| (% control) | | | | | | |
| in T₃ seed | | | | | | |
| p-value | 0.21 | 0.02 | 0.58 | No data | 0.37 | N/A |

*Population mean of the oil content in seed from transgenic lines planted within 30 days of ME11837. Variation is presented as the standard error of the mean.

The oil content in T₃ seed from one event of ME11837 was significantly increased compared to the oil content in corresponding control seed. As presented in Table 22, the oil content was increased to 105% in seed from event −02 compared to the oil content in control seed.

### Example 18

### Results for ME06741 Events

T₂ and T₃ seed from three events and five events, respectively, of ME06741 containing Ceres CLONE ID no. 30018 was analyzed for oil content using FT-NIR spectroscopy as described in Example 2.

The oil content in T₂ seed from one event of ME06741 was significantly increased compared to the mean oil content in seed from transgenic Arabidopsis lines planted within 30 days of ME06741. As presented in Table 23, the oil content was increased to 115% in seed from event −03 compared to the population mean.

TABLE 23
Oil content (% control) in T₂ and T₃ seed from
ME06741 events containing Ceres CLONE ID no. 30018

| | Event- | Event- | Event- | Event- | Event- | |
|---|---|---|---|---|---|---|
| | 01 | 02 | 03 | 04 | 05 | Control |
| Oil content | No data | No data | 115 | 112 | 113 | 100 ± 0* |
| (% control) | | | | | | |
| in T₂ seed | | | | | | |
| p-value | No data | No data | 0.03 | 0.08 | 0.06 | N/A |
| Oil content | 106 ± 4 | 99 ± 1 | 105 ± 1 | 99 ± 3 | 98 ± 2 | 100 ± 1 |
| (% control) | | | | | | |
| in T₃ seed | | | | | | |
| p-value | 0.02 | 0.40 | <0.01 | 0.64 | 0.04 | N/A |

*Population mean of the oil content in seed from transgenic lines planted within 30 days of ME06741. Variation is presented as the standard error of the mean.

The oil content in T₃ seed from two events of ME06741 was significantly increased compared to the oil content in corresponding control seed. As presented in Table 23, the oil content was increased to 106% and 105% in seed from events −01 and −03, respectively, compared to the oil content in control seed. The oil content in T₃ seed from one event of ME06741 was significantly decreased compared to the oil content in corresponding control seed. As presented in Table 23, the oil content was decreased to 98% in seed from event −05 compared to the oil content in control seed.

### Example 19

### Results for ME09515 Events

T₂ and T₃ seed from three events and five events, respectively, of ME09515 containing Ceres ANNOT ID no. 542494 was analyzed for oil content using FT-NIR spectroscopy as described in Example 2.

The oil content in T₂ seed from three events of ME09515 was significantly increased compared to the mean oil content in seed from transgenic Arabidopsis lines planted within 30 days of ME09515. As presented in Table 24, the oil content was increased to 117% in seed from events −02 and −03 and to 116% in seed from event −04 compared to the population mean.

TABLE 24
Oil content (% control) in T₂ and T₃ seed from
ME09515 events containing Ceres ANNOT ID no. 542494

| | Event- | Event- | Event- | Event- | Event- | |
|---|---|---|---|---|---|---|
| | 02 | 03 | 04 | 05 | 07 | Control |
| Oil content | 117 | 117 | 116 | 112 | 108 | 100 ± 0* |
| (% control) | | | | | | |
| in T₂ seed | | | | | | |
| p-value | 0.04 | 0.04 | 0.05 | 0.14 | 0.33 | N/A |
| Oil content | 102 ± 1 | 100 | 95 ± 1 | 98 ± 2 | No data | 100 ± 2 |
| (% control) | | | | | | |
| in T₃ seed | | | | | | |
| p-value | 0.01 | 2.52 | 0.01 | 0.14 | No data | N/A |

*Population mean of the oil content in seed from transgenic lines planted within 30 days of ME09515. Variation is presented as the standard error of the mean.

The oil content in T3 seed from one event of ME09515 was significantly increased compared to the oil content in corresponding control seed. As presented in Table 24, the oil content was increased to 102% in seed from event −02 compared to the oil content in control seed. The oil content in T₃ seed from one event of ME09515 was significantly decreased compared to the oil content in corresponding control seed. As presented in Table 24, the oil content was decreased to 95% in seed from event −04 compared to the oil content in control seed.

### Example 20

### Results for ME09762, ME00874, ME00819, ME07924, and ME08504

A nucleic acid referred to as Ceres CLONE ID no. 945519 was isolated from Brassica napus. Ceres CLONE ID no. 945519 (SEQ ID NO:283) is predicted to encode a 249 amino acid acetyltransferase polypeptide (SEQ ID NO:186) that is a homolog of the polypeptide set forth in SEQ ID NO:185.

The following is a list of nucleic acids that were isolated from Glycine max plants. Ceres CLONE ID no. 690176 (SEQ ID NO:303) is predicted to encode a 479 amino acid cytochrome p450 polypeptide (SEQ ID NO:230) that is a homolog of the polypeptide set forth in SEQ ID NO:229. Ceres CLONE ID no. 574698 (SEQ ID NO:304) is predicted to encode a 472 amino acid cytochrome p450 polypeptide (SEQ ID NO:233) that is also a homolog of the polypeptide set forth in SEQ ID NO:229. Ceres CLONE ID no. 571162 (SEQ ID NO:307) is predicted to encode a 333 amino acid polypeptide (SEQ ID NO:249) that is a homolog of the polypeptide set forth in SEQ ID NO:87.

Each isolated nucleic acid described above was cloned into a Ti plasmid vector, CRS 338, containing a phosphinothricin acetyltransferase gene which confers Finale™ resistance to transformed plants. Constructs were made using CRS 338 that contained Ceres CLONE ID no. 945519, Ceres CLONE ID no. 574698, or Ceres CLONE ID no. 571162, each operably linked to a CaMV 35S promoter. Constructs also were made using CRS 338 that contained Ceres CLONE ID no. 690176 or Ceres CLONE ID no. 574698, each operably linked to a p32449 promoter. Wild-type Arabidopsis plants were transformed separately with each construct. The transformation were performed essentially as described in Bechtold et al., C.R. Acad. Sci. Paris, 316:1194-1199 (1993).

Transgenic Arabidopsis lines containing Ceres CLONE ID no. 945519, Ceres CLONE ID no. 574698, or Ceres CLONE ID no. 571162 operably linked to a CaMV 35S promoter were designated ME09762, ME07924, or ME08504, respectively. Transgenic Arabidopsis lines containing Ceres CLONE I) no. 690176 or Ceres CLONE ID no. 574698 operably linked to a p32449 promoter were designated ME00874 or ME00819, respectively. The presence of each vector containing a DNA clone described above in the respective transgenic Arabidopsis line transformed with the vector was confirmed by Finale™ resistance, PCR amplification from green leaf tissue extract, and/or sequencing of PCR products. As controls, wild-type Arabidopsis plants were transformed with the empty vector CRS 338.

T₂ seed from four events of ME09762 containing Ceres CLONE ID no. 945519 was analyzed for oil content using FT-NIR spectroscopy as described in Example 2. The oil content in T₂ seed from two events of ME09762 was significantly increased compared to the mean oil content in seed from transgenic Arabidopsis lines planted within 30 days of ME09762. As presented in Table 25, the oil content was increased to 116% in seed from events −03 and −04 compared to the population mean.

TABLE 25
Oil content (% control) in T₂ seed from
ME09762 events containing Ceres CLONE ID no. 945519

| | Event-01 | Event-02 | Event-03 | Event-04 | Control |
|---|---|---|---|---|---|
| Oil content | 74 | 103 | 116 | 116 | 100 ± 0* |
| (% control) | | | | | |
| in T₂ seed | | | | | |
| p-value | <0.01 | 0.51 | 0.03 | 0.04 | N/A |

*Population mean of the oil content of seed from transgenic lines planted within 30 days of ME09762.

The oil content in T₂ seed from one event of ME09762 was significantly decreased compared to the mean oil content in seed from transgenic Arabidopsis lines planted within 30 days of ME09762. As presented in Table 25, the oil content was decreased to 74% in seed from event −01 compared to the population mean.

T₂ seed from four events of ME00874 containing Ceres CLONE ID no. 690176 also was analyzed for oil content using FT-NIR spectroscopy as described in Example 2. The oil content in T₂ seed from one event of ME00874 was significantly (p<0.08) decreased compared to the mean oil content in seed from transgenic Arabidopsis lines planted within 30 days of ME00874. As presented in Table 26, the oil content was decreased to 88% in seed from event −03 compared to the population mean.

TABLE 26
Oil content (% control) in T₂ seed
from ME00874 events containing Ceres CLONE ID no. 690176

| | Event-01 | Event-03 | Event-04 | Event-05 | Control |
|---|---|---|---|---|---|
| Oil content | 100 | 88 | 100 | 95 | 100 ± 0* |
| (% control) | | | | | |
| in T₂ seed | | | | | |
| p-value | 0.51 | 0.08 | 0.52 | 0.29 | N/A |

* Population mean of the oil content of seed from transgenic lines planted within 30 days of ME00874.

T₂ seed from one event of ME00819 containing Ceres CLONE ID no. 574698, two events of ME07924 containing Ceres CLONE ID no. 574698, and three events of ME08504 containing Ceres CLONE ID no. 571162 also was analyzed for oil content using FT-NIR spectroscopy as described in Example 2. The oil content in T₂ seed from event −03 of ME00819, events −01 and 10 of ME07924, and events −01, −03, and −04 of ME08504 was not observed to differ significantly from the mean oil content in seed from transgenic Arabidopsis lines planted within 30 days of ME00819, ME07924, and ME08504, respectively. These results are not conclusive, however, since the in planta sequences of the DNA clones used to generate the transgenic plants have not been sequenced, and expression of the encoded polypeptides has not been confirmed.

### Example 21

### Determination of Functional Homolog and/or Ortholog Sequences

A subject sequence was considered a functional homolog or ortholog of a query sequence if the subject and query sequences encoded proteins having a similar function and/or activity. A process known as Reciprocal BLAST (Rivera et al., Proc. Natl. Acad. Sci. USA, 95:6239-6244 (1998)) was used to identify potential functional homolog and/or ortholog sequences from databases consisting of all available public and proprietary peptide sequences, including NR from NCBI and peptide translations from Ceres clones.

Before starting a Reciprocal BLAST process, a specific query polypeptide was searched against all peptides from its source species using BLAST in order to identify polypeptides having BLAST sequence identity of 80% or greater to the query polypeptide and an alignment length of 85% or greater along the shorter sequence in the alignment. The query polypeptide and any of the aforementioned identified polypeptides were designated as a cluster.

The BLASTP version 2.0 program from Washington University at Saint Louis, Mo., USA was used to determine BLAST sequence identity and E-value. The BLASTP version 2.0 program includes the following parameters: 1) an E-value cutoff of 1.0e−5; 2) a word size of 5; and 3) the −postsw option. The BLAST sequence identity was calculated based on the alignment of the first BLAST HSP (High-scoring Segment Pairs) of the identified potential functional homolog and/or ortholog sequence with a specific query polypeptide. The number of identically matched residues in the BLAST HSP alignment was divided by the HSP length, and then multiplied by 100 to get the BLAST sequence identity. The HSP length typically included gaps in the alignment, but in some cases gaps were excluded.

The main Reciprocal BLAST process consists of two rounds of BLAST searches; forward search and reverse search. In the forward search step, a query polypeptide sequence, “polypeptide A,” from source species SA was BLASTed against all protein sequences from a species of interest. Top hits were determined using an E-value cutoff of 10⁻⁵ and a sequence identity cutoff of 35%. Among the top hits, the sequence having the lowest E-value was designated as the best hit, and considered a potential functional homolog or ortholog. Any other top hit that had a sequence identity of 80% or greater to the best hit or to the original query polypeptide was considered a potential functional homolog or ortholog as well. This process was repeated for all species of interest.

In the reverse search round, the top hits identified in the forward search from all species were BLASTed against all protein sequences from the source species SA. A top hit from the forward search that returned a polypeptide from the aforementioned cluster as its best hit was also considered as a potential functional homolog or ortholog.

Functional homologs and/or orthologs were identified by manual inspection of potential functional homolog and/or ortholog sequences. Representative functional homologs and/or orthologs for SEQ ID NO:82, SEQ ID NO:87, SEQ ID NO:148, SEQ ID NO:151, SEQ ID NO:162, SEQ ID NO:175, SEQ ID NO:185, SEQ ID NO:190, SEQ ID NO:198, SEQ ID NO:203, SEQ ID NO:216, SEQ ID NO:229, and SEQ ID NO:245 are shown in FIGS. 1-13, respectively. The percent identities of functional homologs and/or orthologs to SEQ ID NO:82, SEQ ID NO:87, SEQ ID NO:148, SEQ ID NO:151, SEQ ID NO:162, SEQ ID NO:175, SEQ ID NO:185, SEQ ID NO:190, SEQ ID NO:198, SEQ ID NO:203, SEQ ID NO:216, SEQ ID NO:229, and SEQ ID NO:245 are shown below in Tables 27-39, respectively. The BLAST sequence identities and E-values given in Table 27-39 were taken from the forward search round of the Reciprocal BLAST process.

TABLE 27
Percent identity to Ceres CLONE ID no. 625035 (SEQ ID NO: 82)

| | | SEQ | % | | HMM |
|---|---|---|---|---|---|
| | | ID | Iden- | | bit |
| Designation | Species | NO: | tity | e-value | score |
| Ceres CLONE | Glycine max | 82 | N/A | N/A | 571.3 |
| ID no. 625035 | | | | | |
| Public GI no. | Mesembryanthemum | 83 | 69.6 | 1.10E−53 | 689.1 |
| 32401273 | crystallinum | | | | |
| Public GI no. | Oryza sativa | 84 | 55 | 2.60E−36 | 708.5 |
| 14140141 | | | | | |
| Public GI no. | Oryza sativa subsp. | 85 | 55 | 2.60E−36 | 706.3 |
| 50911399 | japonica | | | | |
| Public GI ID | Mesembryanthemum | 341 | 71.4 | 1.70E−73 | 689.1 |
| no. 7528276 | crystallinum | | | | |
| Ceres CLONE | Gossypium hirsutum | 343 | 69.7 | 2.19E−69 | 654.7 |
| ID no. 1926437 | | | | | |

TABLE 28
Percent identity to Ceres CLONE ID no. 5344 (SEQ ID NO: 87)

| | | SEQ ID | % | | HMM bit |
|---|---|---|---|---|---|
| Designation | Species | NO: | Identity | e-value | score |
| Ceres CLONE ID | Arabidopsis thaliana | 87 | N/A | N/A | 817.1 |
| no. 5344 | | | | | |
| Public GI no. | Brassica napus | 88 | 82.7 | 2.29E−149 | 809.4 |
| 26094811 | | | | | |
| Ceres CLONE ID | Brassica napus | 89 | 82.7 | 2.29E−149 | 809.4 |
| no. 1301219 | | | | | |
| Ceres CLONE ID | Zea mays | 90 | 76.5 | 4.00E−136 | 774.7 |
| no. 1411115 | | | | | |
| Public GI no. | Citrus iyo | 91 | 69.5 | 3.29E−116 | 836.5 |
| 3337095 | | | | | |
| Public GI no. | Citrus unshiu | 92 | 69.5 | 5.40E−116 | 836 |
| 3337091 | | | | | |
| Public GI no. | Citrus sp. cv. | 93 | 69.5 | 2.89E−117 | 848.1 |
| 18148925 | sannumphung | | | | |
| Public GI no. | Citrus sp. cv. | 94 | 69.2 | 1.40E−115 | 833.8 |
| 3242641 | sannumphung | | | | |
| Public GI no. | Citrus sinensis | 95 | 69.2 | 1.80E−115 | 831.6 |
| 1617034 | | | | | |
| Public GI no. | Citrus jambhiri | 96 | 69.2 | 3.00E−115 | 830.4 |
| 3205177 | | | | | |
| Public GI no. | Citrus sp. cv. | 97 | 69.2 | 3.70E−117 | 847.5 |
| 18148376 | sannumphung | | | | |
| Public GI no. | Gossypium barbadense | 98 | 69 | 6.20E−115 | 818.7 |
| 33469566 | | | | | |
| Public GI no. | Citrus iyo | 99 | 68.9 | 3.79E−115 | 823 |
| 3337093 | | | | | |
| Public GI no. | Citrus sp. cv. | 100 | 68.9 | 2.00E−116 | 843.2 |
| 18148923 | sannumphung | | | | |
| Public GI no. | Fortunella margarita | 101 | 68.3 | 9.10E−114 | 825.3 |
| 3978580 | | | | | |
| Public GI no. | Poncirus trifoliata | 102 | 68.3 | 6.20E−115 | 832.2 |
| 3978578 | | | | | |
| Public GI no. | Citrus latipes | 103 | 68 | 3.79E−115 | 829.8 |
| 19110472 | | | | | |
| Public GI no. | Citrus hystrix | 104 | 68 | 2.69E−114 | 831.5 |
| 19110474 | | | | | |
| Public GI no. | Citrus aurantiifolia | 105 | 68 | 1.89E−113 | 827.9 |
| 19110478 | | | | | |
| Public GI no. | Citrus jambhiri | 106 | 67.9 | 6.20E−115 | 843.1 |
| 17221624 | | | | | |
| Public GI no. | Citrus jambhiri | 107 | 67.6 | 8.19E−113 | 823.2 |
| 3192102 | | | | | |
| Public GI no. | Citrus jambhiri | 108 | 67.6 | 2.09E−114 | 837.6 |
| 17221626 | | | | | |
| Public GI no. | Prunus persica | 109 | 67.4 | 6.90E−116 | 861.3 |
| 58379364 | | | | | |
| Public GI no. | Microcitrus sp. | 110 | 67.3 | 7.29E−112 | 821.8 |
| 19110476 | citruspark01 | | | | |
| Public GI no. | Actinidia deliciosa | 111 | 67.1 | 5.60E−114 | 823.2 |
| 1143381 | | | | | |
| Public GI no. | Prunus persica | 112 | 67.1 | 1.09E−115 | 860.8 |
| 34068091 | | | | | |
| Public GI no. | Prunus persica | 113 | 67.1 | 3.79E−115 | 852.4 |
| 58379362 | | | | | |
| Public GI no. | Prunus mume | 114 | 66.8 | 8.80E−116 | 856.7 |
| 54306529 | | | | | |
| Public GI no. | Prunus mume | 115 | 66.8 | 1.09E−115 | 856.3 |
| 58379372 | | | | | |
| Public GI no. | Eucalyptus grandis | 116 | 66.4 | 7.19E−105 | 745.1 |
| 6651282 | | | | | |
| Public GI no. | Prunus mahaleb | 117 | 65.9 | 4.39E−114 | 856 |
| 8778050 | | | | | |
| Public GI no. | Prunus americana | 118 | 65.9 | 1.20E−113 | 860.3 |
| 57868641 | | | | | |
| Public GI no. | Prunus salicina | 119 | 65.9 | 3.10E−113 | 854.1 |
| 76365455 | | | | | |
| Public GI no. | Pyrus pyrifolia | 120 | 65.7 | 1.49E−113 | 859.4 |
| 33087508 | | | | | |
| Public GI no. | Eucalyptus grandis | 121 | 65.7 | 1.00E−112 | 853.2 |
| 38234920 | | | | | |
| Public GI no. | Pyrus communis | 122 | 65.7 | 6.39E−113 | 856.4 |
| 33087506 | | | | | |
| Public GI no. | Prunus salicina | 123 | 65.6 | 6.39E−113 | 849.9 |
| 63099931 | | | | | |
| Public GI no. | Pyrus communis | 124 | 65.4 | 1.69E−112 | 851.3 |
| 33087512 | | | | | |
| Public GI no. | Pyrus hybrid cultivar | 125 | 65.4 | 5.69E−112 | 844.1 |
| 33087510 | | | | | |
| Public GI no. | Rubus idaeus | 126 | 65.2 | 2.99E−106 | 817.2 |
| 40732890 | | | | | |
| Public GI no. | Prunus armeniaca | 127 | 65 | 5.69E−112 | 846.2 |
| 2460188 | | | | | |
| Ceres ANNOT ID | Populus balsamifera | 129 | 64.9 | 5.29E−109 | 799 |
| no. 1534757 | subsp. trichocarpa | | | | |
| Public GI no. | Malus x domestica | 130 | 64.8 | 9.40E−112 | 851.6 |
| 1679733 | | | | | |
| Ceres ANNOT ID | Populus balsamifera | 132 | 64.3 | 7.10E−98 | 697.6 |
| no. 1481274 | subsp. trichocarpa | | | | |
| Public GI no. | Vitis vinifera | 133 | 64.1 | 1.09E−108 | 813.7 |
| 21667647 | | | | | |
| Ceres ANNOT ID | Populus balsamifera | 135 | 64.1 | 1.49E−106 | 755.1 |
| no. 1528311 | subsp. trichocarpa | | | | |
| Public GI no. | Lycopersicon | 136 | 64 | 9.99E−108 | 804.2 |
| 469457 | esculentum | | | | |
| Public GI no. | Vitis vinifera | 137 | 63.8 | 1.39E−108 | 798.9 |
| 13172312 | | | | | |
| Public GI no. | Cucumis melo | 138 | 63 | 2.20E−103 | 783.7 |
| 30984105 | | | | | |
| Ceres ANNOT ID | Populus balsamifera | 140 | 62.8 | 2.20E−94 | 668.4 |
| no. 1474878 | subsp. trichocarpa | | | | |
| Public GI no. | Daucus carota | 141 | 58.4 | 7.29E−89 | 716.4 |
| 20066308 | | | | | |
| Public GI no. | Antirrhinum majus | 142 | 56.7 | 3.29E−93 | 768.3 |
| 444011 | | | | | |
| Ceres CLONE ID | Triticum aestivum | 143 | 52.4 | 1.10E−78 | 678.7 |
| no. 784385 | | | | | |
| Public GI no. | Phaseolus vulgaris | 144 | 48.1 | 6.29E−74 | 649.5 |
| 55859509 | | | | | |
| Public GI no. | Phaseolus vulgaris | 145 | 47.3 | 2.50E−72 | 620 |
| 50871748 | | | | | |
| Public GI no. | Phaseolus vulgaris | 146 | 47.3 | 2.50E−72 | 620.3 |
| 55859507 | | | | | |
| Ceres CLONE ID | Glycine max | 249 | 61.6 | 1.4E−103 | 798.5 |
| no. 571162 | | | | | |
| Public GI ID no. | Gossypium barbadense | 344 | 69 | 7.79E−115 | 818.7 |
| 33469564 | | | | | |
| Ceres CLONE ID | Panicum virgatum | 346 | 47.1 | 5.60E−73 | 588.3 |
| no. 1820701 | | | | | |

TABLE 29
Percent identity to Ceres CDNA ID no. 23649975 (SEQ ID NO: 148)

| | | SEQ | | | HMM |
|---|---|---|---|---|---|
| | | ID | % | | bit |
| Designation | Species | NO: | Identity | e-value | score |
| Ceres CDNA ID | Arabidopsis | 148 | N/A | N/A | 473.9 |
| no. 23649975 | thaliana | | | | |
| Ceres CLONE | Brassica napus | 149 | 83 | 3.30E−61 | 468.6 |
| ID no. 948978 | | | | | |

TABLE 30
Percent identity to Ceres CDNA ID no. 12703936 (SEQ ID NO: 151)

| | | SEQ ID | % | e- | HMM bit |
|---|---|---|---|---|---|
| Designation | Species | NO: | Identity | value | score |
| Ceres CDNA ID no. | Arabidopsis thaliana | 151 | N/A | N/A | 477.3 |
| 12703936 | | | | | |
| Ceres ANNOT ID no. | Populus balsamifera | 153 | 74.5 | 9.80E−85 | 487.7 |
| 1488415 | subsp. trichocarpa | | | | |
| Ceres ANNOT ID no. | Populus balsamifera | 155 | 73.2 | 7.70E−85 | 458.2 |
| 1460393 | subsp. trichocarpa | | | | |
| Ceres CLONE ID no. | Glycine max | 156 | 68.8 | 5.99E−78 | 471.6 |
| 524650 | | | | | |
| Ceres CLONE ID no. | Zea mays | 157 | 68.4 | 5.40E−77 | 487.7 |
| 237720 | | | | | |
| Ceres CLONE ID no. | Zea mays | 158 | 68 | 4.90E−67 | 426.4 |
| 465517 | | | | | |
| Ceres CLONE ID no. | Triticum aestivum | 159 | 63.7 | 8.49E−63 | 428.4 |
| 703914 | | | | | |
| Public GI no. | Oryza sativa subsp. | 160 | 62.1 | 1.99E−63 | 442.2 |
| 50881429 | japonica | | | | |
| Ceres CLONE ID no. | Panicum virgatum | 348 | 72.4 | 3.10E−79 | 477.9 |
| 1817099 | | | | | |
| Ceres CLONE ID no. | Gossypium hirsutum | 350 | 71.9 | 1.70E−78 | 411.5 |
| 1808214 | | | | | |
| Ceres CLONE ID no. | Gossypium hirsutum | 352 | 70.9 | 3.70E−76 | 396.9 |
| 1870041 | | | | | |
| Public GI ID no. | Oryza sativa subsp. | 353 | 69.2 | 1.40E−78 | 483.6 |
| 108862961 | japonica | | | | |

TABLE 31
Percent identity to Ceres CDNA ID no. 12706677 (SEQ ID NO: 162)

| | | SEQ ID | % | e- | HMM bit |
|---|---|---|---|---|---|
| Designation | Species | NO: | Identity | value | score |
| Ceres CDNA ID no. | Arabidopsis thaliana | 162 | N/A | N/A | 431.4 |
| 12706677 | | | | | |
| Ceres CLONE ID no. | Brassica napus | 163 | 87.5 | 3.70E−78 | 440.8 |
| 952316 | | | | | |
| Ceres CLONE ID no. | Glycine max | 164 | 72.4 | 3.30E−61 | 438 |
| 649261 | | | | | |
| Ceres ANNOT ID no. | Populus balsamifera | 166 | 69.6 | 1.19E−58 | 433.3 |
| 1469350 | subsp. trichocarpa | | | | |
| Ceres ANNOT ID no. | Populus balsamifera | 168 | 68.75 | 6.19E−60 | 423.7 |
| 1488942 | subsp. trichocarpa | | | | |
| Ceres CLONE ID no. | Zea mays | 169 | 67.6 | 2.10E−52 | 420.4 |
| 234461 | | | | | |
| Ceres CLONE ID no. | Zea mays | 170 | 66.6 | 5.49E−52 | 415 |
| 217678 | | | | | |
| Ceres CLONE ID no. | Triticum aestivum | 171 | 65.2 | 6.29E−51 | 419.1 |
| 1327188 | | | | | |
| Ceres CLONE ID no. | Gossypium hirsutum | 355 | 75.1 | 3.09E−63 | 416.3 |
| 1831965 | | | | | |
| Ceres CLONE ID no. | Panicum virgatum | 357 | 67 | 6.60E−54 | 406.1 |
| 1770078 | | | | | |
| Ceres CLONE ID no. | Panicum virgatum | 359 | 65.2 | 2.19E−48 | 373.2 |
| 2008759 | | | | | |

TABLE 32
Percent identity to Ceres ANNOT ID no. 542494 (SEQ ID NO: 175)

| | | SEQ ID | % | e- | HMM bit |
|---|---|---|---|---|---|
| Designation | Species | NO: | Identity | value | score |
| Ceres ANNOT ID no. | Arabidopsis thaliana | 175 | N/A | N/A | 371.3 |
| 542494 | | | | | |
| Ceres CLONE ID no. | Zea mays | 176 | 89.1 | 1.19E−63 | 356.6 |
| 1369396 | | | | | |
| Ceres CLONE ID no. | Brassica napus | 177 | 85.4 | 1.70E−53 | 291.9 |
| 1102549 | | | | | |
| Ceres ANNOT ID no. | Populus balsamifera | 179 | 72.7 | 1.70E−53 | 370.3 |
| 1515577 | subsp. trichocarpa | | | | |
| Ceres CLONE ID no. | Glycine max | 180 | 70 | 6.10E−51 | 362.1 |
| 516401 | | | | | |
| Ceres CLONE ID no. | Triticum aestivum | 181 | 45.1 | 8.59E−29 | 347 |
| 618542 | | | | | |
| Public GI no. | Oryza sativa subsp. | 182 | 44.8 | 1.39E−26 | 348.7 |
| 50940451 | japonica | | | | |
| Ceres CLONE ID no. | Zea mays | 183 | 42.8 | 9.79E−28 | 330.2 |
| 305154 | | | | | |
| Ceres CLONE ID no. | Panicum virgatum | 309 | 44.3 | 2.89E−28 | 338.8 |
| 1779106 | | | | | |

TABLE 33
Percent identity to Ceres ANNOT ID no. 549258 (SEQ ID NO: 185)

| | | SEQ | | | HMM |
|---|---|---|---|---|---|
| | | ID | % | | bit |
| Designation | Species | NO: | Identity | e-value | score |
| Ceres ANNOT | Arabidopsis | 185 | N/A | N/A | 731.2 |
| ID no. 549258 | thaliana | | | | |
| Ceres CLONE | Brassica napus | 186 | 87.4 | 8.39E−109 | 703.1 |
| ID no. 945519 | | | | | |
| Public GI no. | Oryza sativa | 187 | 60.8 | 170E−55 | 798.4 |
| 50935585 | subsp. japonica | | | | |
| Public GI no. | Oryza sativa | 188 | 60.8 | 1.70E−55 | 798.4 |
| 51963354 | subsp. japonica | | | | |

TABLE 34
Percent identity to Ceres ANNOT ID no. 564261 (SEQ ID NO: 190)

| | | SEQ ID | % | | HMM bit |
|---|---|---|---|---|---|
| Designation | Species | NO: | Identity | e-value | score |
| Ceres ANNOT ID no. | Arabidopsis thaliana | 190 | N/A | N/A | 667.5 |
| 564261 | | | | | |
| Ceres CLONE ID no. | Brassica napus | 191 | 83.1 | 1.90E−104 | 649.9 |
| 947761 | | | | | |
| Ceres CLONE ID no. | Glycine max | 192 | 64.7 | 3.10E−72 | 607.9 |
| 680759 | | | | | |
| Public GI no. | Oryza sativa subsp. | 193 | 64.3 | 8.49E−61 | 643.4 |
| 77549263 | japonica | | | | |
| Ceres ANNOT ID no. | Populus balsamifera | 195 | 62.7 | 6.00E−76 | 658.1 |
| 1486789 | subsp. trichocarpa | | | | |
| Ceres CLONE ID no. | Zea mays | 196 | 61.3 | 1.90E−63 | 642.9 |
| 230678 | | | | | |
| Ceres CLONE ID no. | Musa acuminata | 311 | 65.8 | 8.69E−68 | 602 |
| 1715450 | | | | | |
| Ceres CLONE ID | Panicum virgatum | 313 | 62.6 | 4.59E−62 | 618.1 |
| no. 1763963 | | | | | |
| Ceres CLONE ID no. | Gossypium hirsutum | 315 | 62 | 6.90E−75 | 654 |
| 1849790 | | | | | |
| Ceres CLONE ID | Panicum virgatum | 317 | 60.6 | 2.49E−63 | 640.8 |
| no. 1795526 | | | | | |

TABLE 35
Percent identity to Ceres ANNOT ID no. 565548 (SEQ ID NO: 198)

| | | SEQ | | | HMM |
|---|---|---|---|---|---|
| | | ID | % | | bit |
| Designation | Species | NO: | Identity | e-value | score |
| Ceres ANNOT ID | Arabidopsis | 198 | N/A | N/A | 622.1 |
| no. 565548 | thaliana | | | | |
| Ceres CLONE ID | Brassica napus | 199 | 66.3 | 4.40E−57 | 559.4 |
| no. 976147 | | | | | |

TABLE 36
Percent identity to Ceres CLONE ID no. 2721 (SEQ ID NO: 203)

| | | SEQ | | | HMM |
|---|---|---|---|---|---|
| | | ID | % | | bit |
| Designation | Species | NO: | Identity | e-value | score |
| Ceres CLONE ID | Arabidopsis | 203 | N/A | N/A | 323.8 |
| no. 2721 | thaliana | | | | |
| Ceres CLONE ID | Brassica napus | 204 | 82.8 | 6.80E−52 | 307 |
| no. 871180 | | | | | |
| Ceres CLONE ID | Glycine max | 205 | 80.1 | 1.49E−40 | 212.4 |
| no. 1115650 | | | | | |
| Ceres CLONE ID | Panicum | 206 | 79.1 | 1.60E−25 | 278.8 |
| no. 1767185 | virgatum | | | | |
| Public GI no. | Spinacia | 207 | 79 | 1.39E−28 | 297.9 |
| 1617213 | oleracea | | | | |
| Ceres CLONE ID | Triticum | 208 | 78.6 | 1.50E−24 | 278.7 |
| no. 772741 | aestivum | | | | |
| Ceres CLONE ID | Panicum | 209 | 77.7 | 3.40E−25 | 268.5 |
| no. 1760834 | virgatum | | | | |
| Ceres CLONE ID | Panicum | 210 | 77.7 | 4.40E−25 | 266.6 |
| no. 1762311 | virgatum | | | | |
| Ceres CLONE ID | Brassica napus | 211 | 77.5 | 9.40E−39 | 174 |
| no. 1080241 | | | | | |
| Ceres CLONE ID | Brassica napus | 212 | 77.2 | 1.29E−41 | 248.7 |
| no. 960043 | | | | | |
| Ceres CLONE ID | Panicum | 213 | 76.3 | 9.10E−25 | 260.6 |
| no. 1782555 | virgatum | | | | |
| Ceres CLONE ID | Brassica napus | 214 | 76.1 | 2.40E−49 | 290.4 |
| no. 1036232 | | | | | |
| Public GI ID | Pisum sativum | 318 | 64.2 | 2.80E−32 | 315.9 |
| no. 1617206 | | | | | |
| Ceres CLONE ID | Gossypium | 320 | 63.1 | 2.40E−33 | 310.2 |
| no. 1808894 | hirsutum | | | | |
| Public GI ID no. | Nicotiana | 321 | 57.3 | 4.60E−30 | 317.6 |
| 1617197 | tabacum | | | | |

TABLE 37
Percent identity to Ceres CLONE ID no. 30018 (SEQ ID NO: 216)

| | | | | | HMM |
|---|---|---|---|---|---|
| | | SEQ ID | % | e- | bit |
| Designation | Species | NO: | Identity | value | score |
| Ceres CLONE ID no. | Arabidopsis thaliana | 216 | N/A | N/A | 176.1 |
| 30018 | | | | | |
| Ceres ANNOT ID no. | Populus balsamifera | 218 | 81.6 | 2.29E−26 | 168.8 |
| 1488347 | subsp. trichocarpa | | | | |
| Ceres ANNOT ID no. | Populus balsamifera | 220 | 76.3 | 9.10E−25 | 168.5 |
| 1513719 | subsp. trichocarpa | | | | |
| Public GI no. 633685 | Solanum tuberosum | 221 | 76.3 | 1.50E−24 | 164 |
| Ceres CLONE ID no. | Glycine max | 222 | 76 | 8.19E−24 | 168.7 |
| 853331 | | | | | |
| Ceres CLONE ID no. | Zea mays | 223 | 74.1 | 8.40E−22 | 168.3 |
| 208991 | | | | | |
| Ceres CLONE ID no. | Zea mays | 224 | 72.5 | 2.19E−21 | 169 |
| 336493 | | | | | |
| Ceres CLONE ID no. | Zea mays | 225 | 72.5 | 5.89E−21 | 160.9 |
| 1064967 | | | | | |
| Ceres CLONE ID no. | Triticum aestivum | 226 | 72.5 | 5.89E−21 | 160.9 |
| 639802 | | | | | |
| Public GI no. 4775284 | Chlorella protothecoides | 227 | 58.3 | 7.70E−12 | 132.2 |
| Ceres CLONE ID no. | Brassica napus | 323 | 92.3 | 2.59E−27 | 170.5 |
| 959117 | | | | | |
| Ceres CLONE ID no. | Brassica napus | 325 | 90.7 | 3.40E−27 | 169.2 |
| 1090391 | | | | | |
| Ceres CLONE ID no. | Brassica napus | 327 | 89.8 | 4.00E−24 | 165.2 |
| 1270157 | | | | | |
| Ceres CLONE ID no. | Gossypium hirsutum | 329 | 81.9 | 5.50E−27 | 173.1 |
| 1797853 | | | | | |
| Ceres CLONE ID no. | Papaver somniferum | 331 | 80.6 | 1.59E−20 | 163.9 |
| 1620853 | | | | | |
| Public GI ID no. | Medicago truncatula | 332 | 80.5 | 3.89E−26 | 173.6 |
| 92867670 | | | | | |
| Ceres CLONE ID no. | Panicum virgatum | 334 | 77.4 | 1.99E−22 | 172.8 |
| 1955598 | | | | | |
| Public GI ID no. | Solanum tuberosum | 335 | 76.3 | 1.89E−24 | 164 |
| 1174870 | | | | | |
| Ceres CLONE ID no. | Musa acuminata | 337 | 70.5 | 7.70E−21 | 159.5 |
| 1739308 | | | | | |

TABLE 38
Percent identity to Ceres CLONE ID no. 36334 (SEQ ID NO: 229)

| | | SEQ ID | % | | HMM bit |
|---|---|---|---|---|---|
| Designation | Species | NO: | Identity | e-value | score |
| Ceres CLONE ID no. | Arabidopsis thaliana | 229 | N/A | N/A | 1125 |
| 36334 | | | | | |
| Ceres CLONE ID no. | Glycine max | 230 | 79.1 | 0 | 1127 |
| 690176 | | | | | |
| Ceres ANNOT ID no. | Populus balsamifera | 232 | 79 | 2.09E−198 | 1132 |
| 1464715 | subsp. trichocarpa | | | | |
| Ceres CLONE ID no. | Glycine max | 233 | 78.1 | 0 | 1085 |
| 574698 | | | | | |
| Public GI no. 9587211 | Vigna radiata | 234 | 77.9 | 0 | 1124 |
| Ceres CLONE ID no. | Glycine max | 235 | 77.8 | 0 | 1093 |
| 718939 | | | | | |
| Ceres ANNOT ID no. | Populus balsamifera | 237 | 77.7 | 1.49E−197 | 1107 |
| 1511511 | subsp. trichocarpa | | | | |
| Public GI no. | Nicotiana tabacum | 238 | 75.4 | 0 | 1101 |
| 45260636 | | | | | |
| Public GI no. | Artemisia annua | 239 | 72 | 5.49E−176 | 1078 |
| 86279652 | | | | | |
| Public GI no. | Zinnia elegans | 240 | 70.7 | 1.10E−168 | 997.6 |
| 71834072 | | | | | |
| Public GI no. | Oryza sativa subsp. | 241 | 64 | 0 | 1069 |
| 60677685 | japonica | | | | |
| Ceres CLONE ID no. | Zea mays | 242 | 63.1 | 0 | 1046 |
| 339347 | | | | | |
| Public GI no. | Oryza sativa subsp. | 243 | 62.6 | 3.40E−144 | 1059 |
| 77548615 | japonica | | | | |
| Public GI ID no. | Citrus sinensis | 338 | 80.3 | 4.89E−200 | 1129.2 |
| 70609692 | | | | | |
| Ceres CLONE ID no. | Panicum virgatum | 340 | 62.1 | 7.19E−144 | 1064.1 |
| 1786280 | | | | | |

TABLE 39
Percent identity to Ceres CLONE ID no. 37493 (SEQ ID NO: 245)

| | | SEQ | | | HMM |
|---|---|---|---|---|---|
| | | ID | % | | bit |
| Designation | Species | NO: | Identity | e-value | score |
| Ceres CLONE | Arabidopsis | 245 | N/A | N/A | 1008 |
| ID no. 37493 | thaliana | | | | |
| Ceres ANNOT | Populus | 247 | 76.5 | 3.00E−156 | 1012 |
| ID no. 1494370 | balsamifera | | | | |
| | subsp. | | | | |
| | trichocarpa | | | | |
| Public GI no. | Oryza sativa | 248 | 65.5 | 8.49E−127 | 1035 |
| 50929439 | subsp. japonica | | | | |

### Example 22

### Generation of Hidden Markov Models

Hidden Markov Models (HMMs) were generated by the program HMMER 2.3.2 using groups of sequences as input that are homologous and/or orthologous to each of SEQ ID NO:80, SEQ ID NO:414, SEQ ID NO:82, SEQ ID NO:87, SEQ ID NO:148, SEQ ID NO:151, SEQ ID NO:162, SEQ ID NO:175, SEQ ID NO:185, SEQ ID NO:190, SEQ ID NO:198, SEQ ID NO:203, SEQ ID NO:216, SEQ ID NO:229, and SEQ ID NO:245. To generate each HMM, the default HMMER 2.3.2 program parameters configured for glocal alignments were used.

An HMM was generated using the following sequences as input: SEQ ID NO:80, SEQ ID NOs:415-418, SEQ ID NOs:420-431, SEQ ID NOs:433-434, SEQ ID NO:437, and SEQ ID NOs:439-441. The sequences are aligned in FIG. 14. When fitted to the HMM, the sequences had the HMM bit scores listed in Table 40. Other homologous and/or orthologous sequences include SEQ ID NO:419, SEQ ID NO:432, SEQ ID NOs:435-436, and SEQ ID NO:438. These sequences also were fitted to the HMM, and the HMM bit scores are listed in Table 40.

TABLE 40
HMM bit scores of sequences related to SEQ ID NO: 80

| | | SEQ ID | HMM bit |
|---|---|---|---|
| Designation | Species | NO: | score |
| Ceres CLONE ID no. 590462 | Artificial | 80 | 147.6 |
| | sequence | | |
| Public GI ID no. 114974_T | Artificial | 415 | 124.1 |
| | sequence | | |
| Public GI ID no. 92881003_T | Artificial | 416 | 131.4 |
| | sequence | | |
| Public GI ID no. 54290938_T | Artificial | 417 | 121.7 |
| | sequence | | |
| Public GI ID no. 16757966_T | Artificial | 418 | 129.9 |
| | sequence | | |
| Ceres ANNOT ID no. | Artificial | 419 | 126.8 |
| 1490788_T | sequence | | |
| Public GI ID no. 54401705_T | Artificial | 420 | 132.3 |
| | sequence | | |
| Ceres ANNOT ID no. | Artificial | 421 | 126.8 |
| 1437978_T | sequence | | |
| Public GI ID no. 6118076_T | Artificial | 422 | 141.3 |
| | sequence | | |
| Public GI ID no. 32400332_T | Artificial | 423 | 132.7 |
| | sequence | | |
| Public GI ID no. 110623260_T | Artificial | 424 | 129.8 |
| | sequence | | |
| Ceres CLONE ID no. | Artificial | 425 | 129.7 |
| 1777157_T | sequence | | |
| Ceres CLONE ID no. 732610_T | Artificial | 426 | 132.1 |
| | sequence | | |
| Ceres CLONE ID no. | Artificial | 427 | 114 |
| 1926430_T | sequence | | |
| Public GI ID no. 6840855_T | Artificial | 428 | 104.4 |
| | sequence | | |
| Ceres CLONE ID no. 327253_T | Artificial | 429 | 125.5 |
| | sequence | | |
| Public GI ID no. 249262_T | Artificial | 430 | 110.3 |
| | sequence | | |
| Public GI ID no. 28628597_T | Artificial | 431 | 112.4 |
| | sequence | | |
| Public GI ID no. 11034736_T | Artificial | 432 | 129.5 |
| | sequence | | |
| Public GI ID no. 127734_T | Artificial | 433 | 124.5 |
| | sequence | | |
| Public GI ID no. 17226270_T | Artificial | 434 | 133.8 |
| | sequence | | |
| Public GI ID no. 62131643_T | Artificial | 435 | 125.4 |
| | sequence | | |
| Public GI ID no. 56130951_T | Artificial | 436 | 125.6 |
| | sequence | | |
| Public GI ID no. 127733_T | Artificial | 437 | 123.5 |
| | sequence | | |
| Public GI ID no. 12621052_T | Artificial | 438 | 121.9 |
| | sequence | | |
| Public GI ID no. 71361195_T | Artificial | 439 | 128.6 |
| | sequence | | |
| Public GI ID no. 56112345_T | Artificial | 440 | 125.5 |
| | sequence | | |
| Public GI ID no. 11034734_T | Artificial | 441 | 133 |
| | sequence | | |

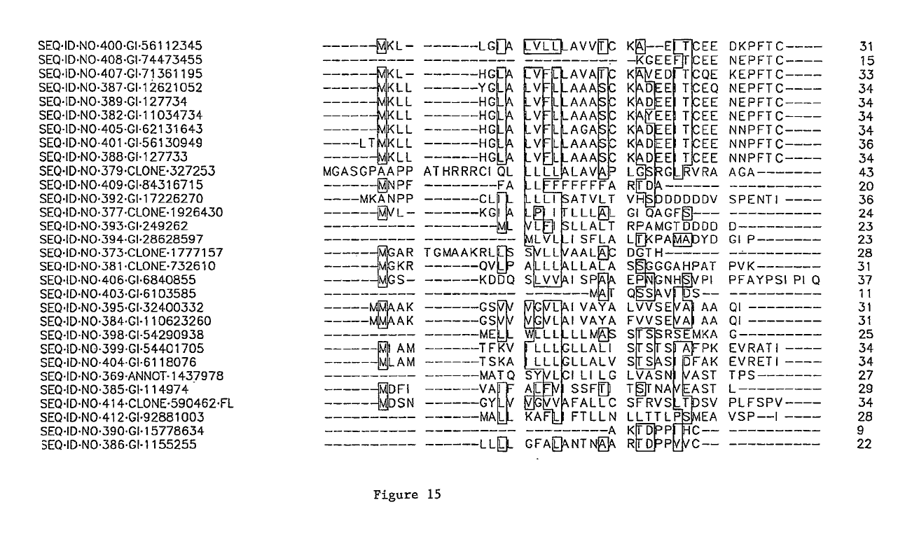

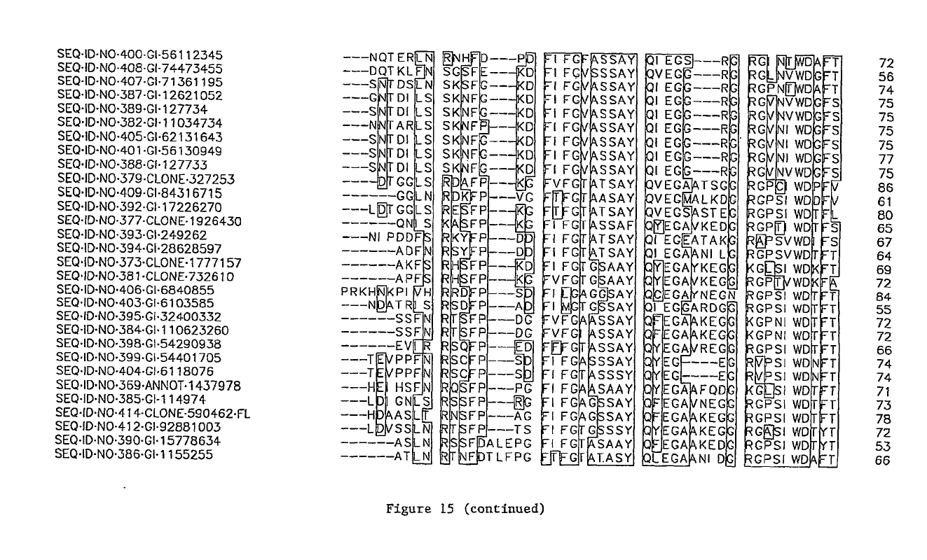

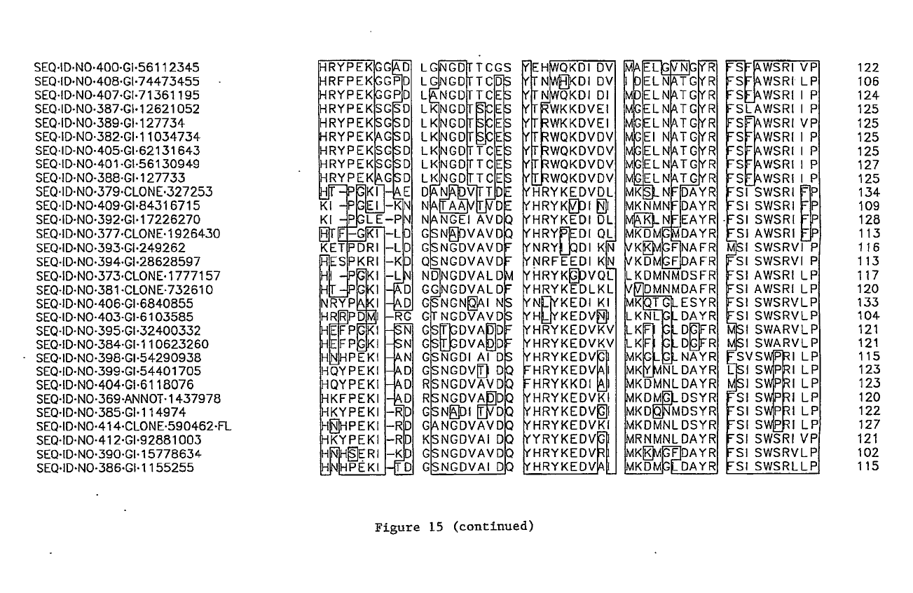

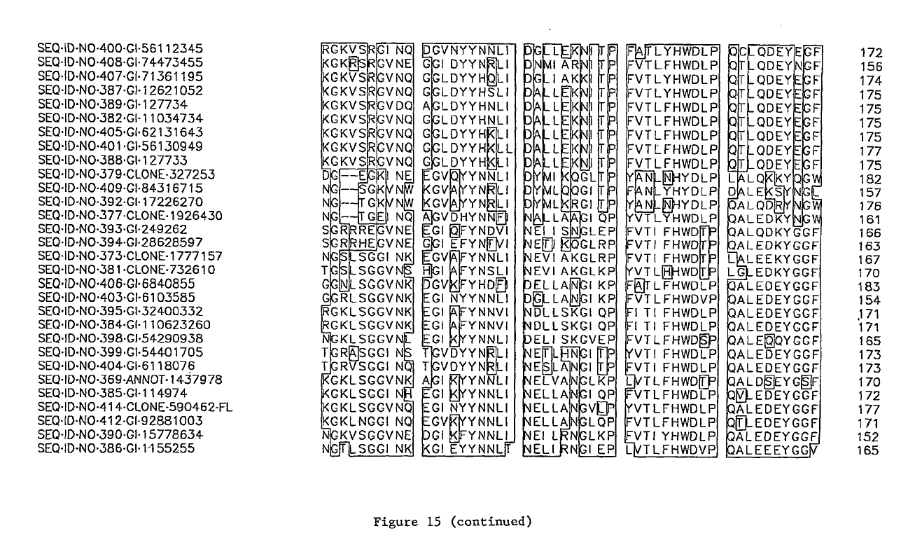

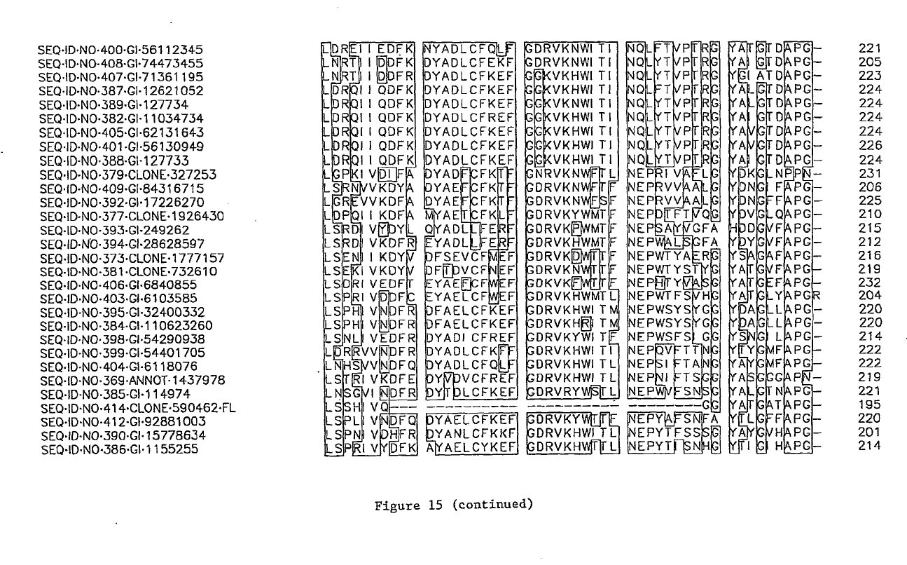

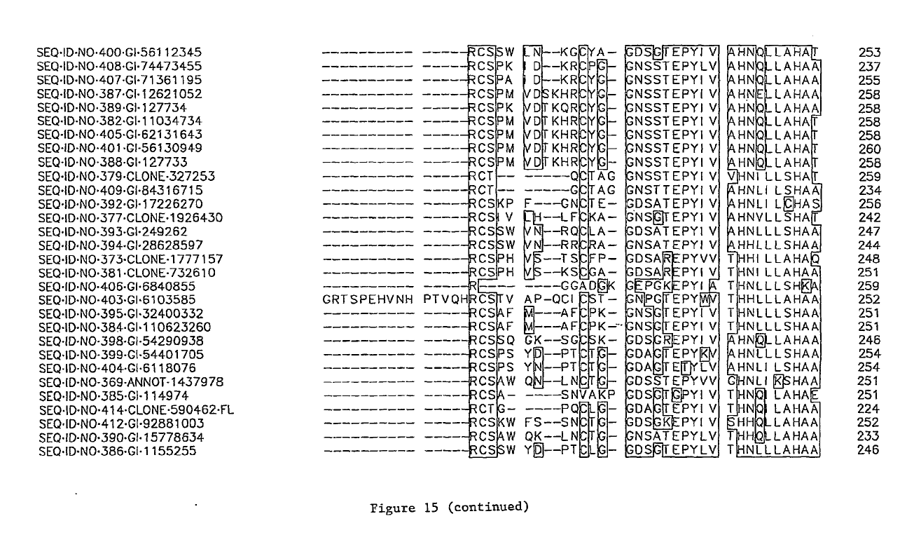

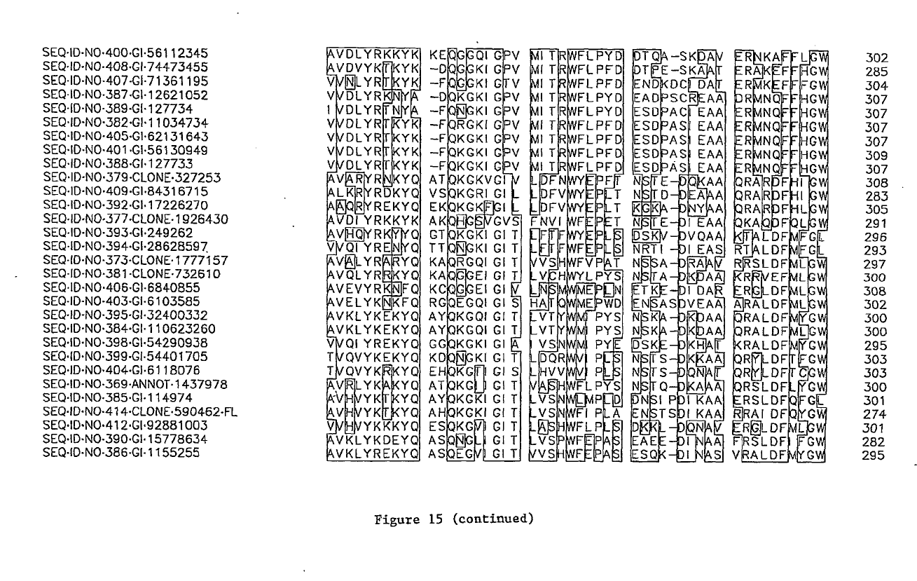

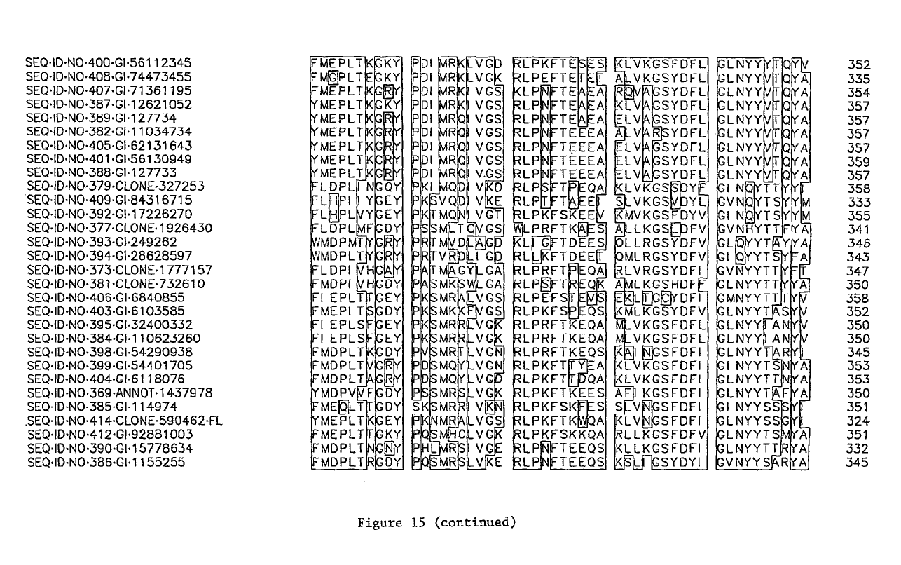

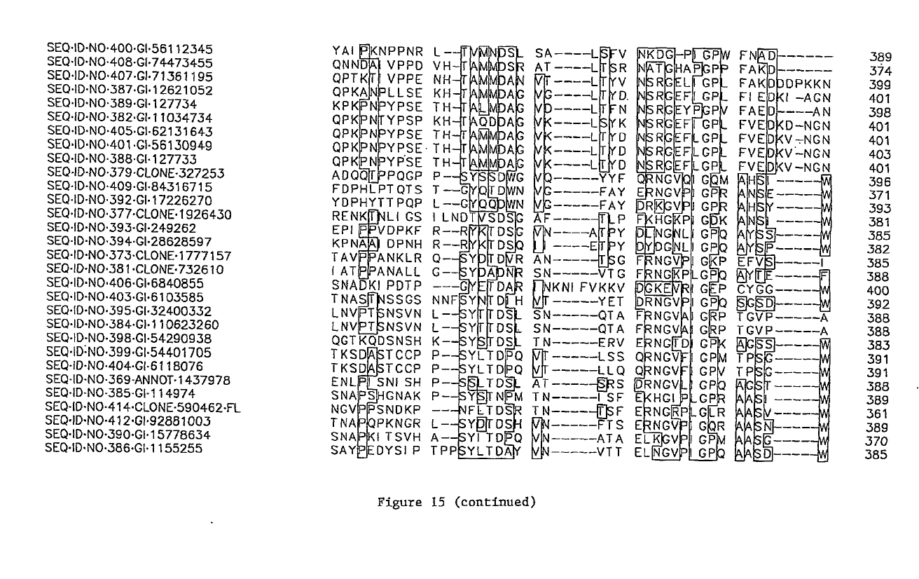

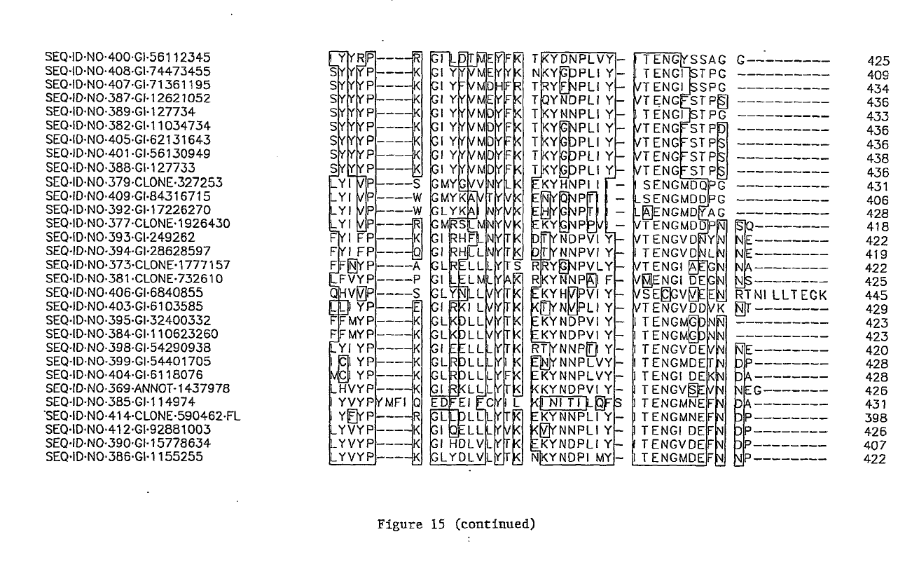

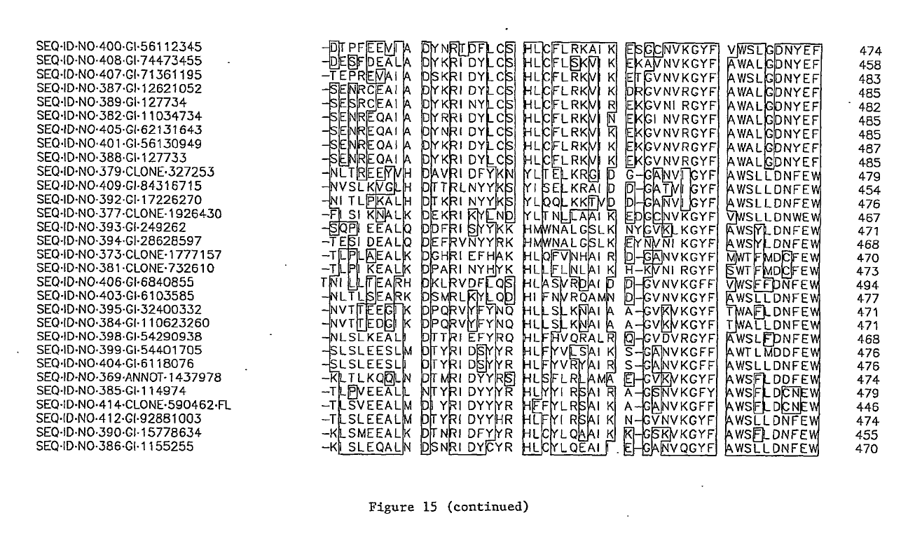

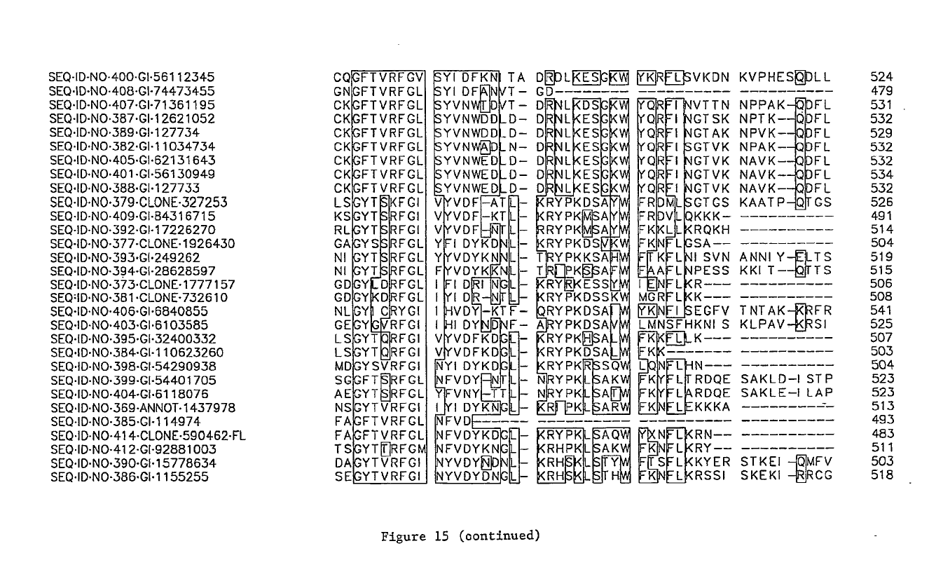

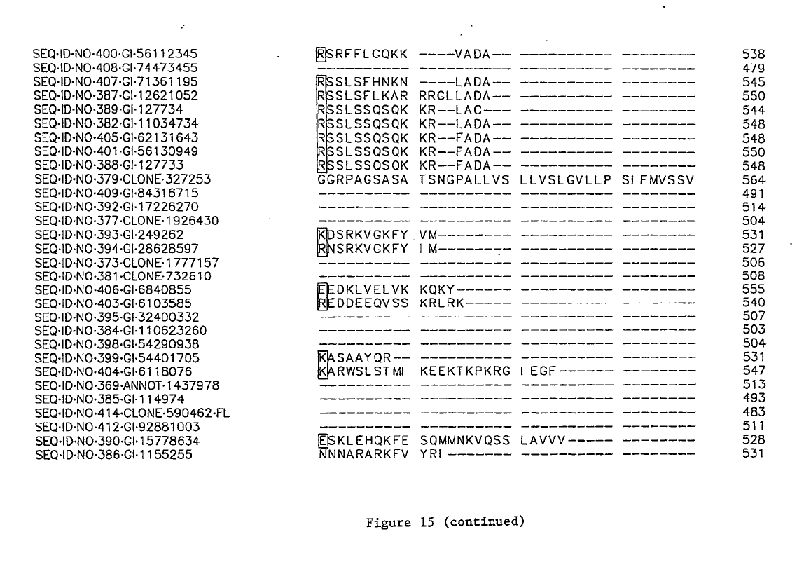

An HMM was generated using the following sequences as input: SEQ ID NO:414, SEQ ID NO:369, SEQ ID NO:373, SEQ ID NO:377, SEQ ID NO:379, SEQ ID NOs:381-382, SEQ ID NOs:384-390, SEQ ID NOs:392-395, SEQ ID NOs:398-401, SEQ ID NOs:403-409, and SEQ ID NO:412. The sequences are aligned in FIG. 15. When fitted to the , the sequences had the HMM bit scores listed in Table 41. Other homologous and/or orthologous sequences include SEQ ID NO:371, SEQ ID NO:375, SEQ ID NO:383, SEQ ID NO:391, SEQ ID NOs:396-397, SEQ ID NO:402, and SEQ ID NOs:410-411. These sequences also were fitted to the HMM, and the HMM bit scores are listed in Table 41.

TABLE 41
HMM bit scores of sequences related to SEQ ID NO: 414

| | | SEQ | HMM |
|---|---|---|---|
| | | ID | bit |
| Designation | Species | NO: | score |
| Ceres CLONE ID no. | Glycine max | 414 | 1206.1 |
| 590462_FL | | | |
| Ceres ANNOT ID no. | Populus balsamifera subsp. | 369 | 1318 |
| 1437978 | trichocarpa | | |
| Ceres ANNOT ID no. | Populus balsamifera subsp. | 371 | 1213.9 |
| 1490788 | trichocarpa | | |
| Ceres CLONE ID no. | Panicum virgatum | 373 | 1211.1 |
| 1777157 | | | |
| Ceres CLONE ID no. | Panicum virgatum | 375 | 1120.6 |
| 1792940 | | | |
| Ceres CLONE ID no. | Gossypium hirsutum | 377 | 1210.8 |
| 1926430 | | | |
| Ceres CLONE ID no. | Zea mays | 379 | 1176.8 |
| 327253 | | | |
| Ceres CLONE ID no. | Triticum aestivum | 381 | 1237.7 |
| 732610 | | | |
| Public GI ID no. | Raphanus sativus | 382 | 1255.8 |
| 11034734 | | | |
| Public GI ID no. | Raphanus sativus | 383 | 1272.3 |
| 11034736 | | | |
| Public GI ID no. | Camellia sinensis | 384 | 1258 |
| 110623260 | | | |
| Public GI ID no. 114974 | Trifolium repens | 385 | 1123.9 |
| Public GI ID no. | Prunus avium | 386 | 1297.4 |
| 1155255 | | | |
| Public GI ID no. | Brassica juncea | 387 | 1238.7 |
| 12621052 | | | |
| Public GI ID no. 127733 | Brassica napus | 388 | 1267.3 |
| Public GI ID no. 127734 | Sinapis alba | 389 | 1252.4 |
| Public GI ID no. | Prunus serotina | 390 | 1317.2 |
| 15778634 | | | |
| Public GI ID no. | Prunus serotina | 391 | 1348 |
| 16757966 | | | |
| Public GI ID no. | Lycopersicon esculentum | 392 | 1189.3 |
| 17226270 | | | |
| Public GI ID no. 249262 | Manihot esculenta | 393 | 1205.9 |
| Public GI ID no. | Hevea brasiliensis | 394 | 1231.4 |
| 28628597 | | | |
| Public GI ID no. | Camellia sinensis | 395 | 1285.1 |
| 32400332 | | | |
| Public GI ID no. | Brassica juncea | 396 | 1237.1 |
| 4033345 | | | |
| Public GI ID no. | Brassica napus | 397 | 1227.7 |
| 414103 | | | |
| Public GI ID no. | Oryza sativa subsp. japonica | 398 | 1310.9 |
| 54290938 | | | |
| Public GI ID no. | Dalbergia nigrescens | 399 | 1327.7 |
| 54401705 | | | |
| Public GI ID no. | Armoracia rusticana | 400 | 1224.9 |
| 56112345 | | | |
| Public GI ID no. | Brassica rapa subsp. | 401 | 1263.7 |
| 56130949 | pekinensis | | |
| Public GI ID no. | Brassica rapa subsp. | 402 | 1263.2 |
| 56130951 | pekinensis | | |
| Public GI ID no. | Rauvolfia serpentina | 403 | 1254.7 |
| 6103585 | | | |
| Public GI ID no. | Dalbergia cochinchinensis | 404 | 1322.3 |
| 6118076 | | | |
| Public GI ID no. | Brassica rapa var. | 405 | 1265.2 |
| 62131643 | parachinensis | | |
| Public GI ID no. | Catharanthus roseus | 406 | 1159.2 |
| 6840855 | | | |
| Public GI ID no. | Wasabia japonica | 407 | 1244.9 |
| 71361195 | | | |
| Public GI ID no. | Arabidopsis lyrata subsp. | 408 | 1112.2 |
| 74473455 | lyrata | | |
| Public GI ID no. | Oncidium cv. ‘Gower | 409 | 1136.6 |
| 84316715 | Ramsey’ | | |
| Public GI ID no. | Oncidium cv. ‘Gower | 410 | 1121.9 |
| 84316796 | Ramsey’ | | |
| Public GI ID no. | Oncidium cv. ‘Gower | 411 | 1136.5 |
| 84316817 | Ramsey’ | | |
| Public GI ID no. | Medicago truncatula | 412 | 1369.4 |
| 92881003 | | | |

An HMM was generated using the following sequences as input: SEQ ID NOs:82-84 and SEQ ID NO:343. The sequences are aligned in FIG. 1. When fitted to the HMM, the sequences had the HMM bit scores listed in Table 27. Other homologous and/or orthologous sequences also were fitted to the HMM, and these sequences are listed in Table 27 along with their corresponding HMM bit scores.

An HMM was generated using the following sequences as input: SEQ ID NOs:87-88, SEQ ID NOs:90-93, SEQ ID NOs:95-96, SEQ ID NO:98, SEQ ID NOs:101-106, SEQ ID NOs:109-112, SEQ ID NO:114, SEQ ID NO:117-122, SEQ ID NO:124, SEQ ID NOs:126-127, SEQ ID NOs:129-130, SEQ ID NO:133, SEQ ID NOs:136-138, SEQ ID NOs:141-144, and SEQ ID NO:249. The sequences are aligned in FIG. 2. When fitted to the HMM, the sequences had the HMM bit scores listed in Table 28. Other homologous and/or orthologous sequences also were fitted to the HMM, and these sequences are listed in Table 28 along with their corresponding HMM bit scores.

An HMM was generated using the following sequences as input: SEQ ID NO:360 and SEQ ID NO:149. The sequences are aligned in FIG. 3. When fitted to the HMM, SEQ ID NO:148 and SEQ ID NO:149 had the HMM bit scores listed in Table 29. SEQ ID NO:360 had an HMM bit score of 473.9 when fitted to the HMM.

An HMM was generated using the following sequences as input: SEQ ID NO:151, SEQ ID NO:153, SEQ ID NOs:156-157, and SEQ ID NOs:159-160. The sequences are aligned in FIG. 4. When fitted to the HMM, the sequences had the HMM bit scores listed in Table 30. Other homologous and/or orthologous sequences also were fitted to the HMM, and these sequences are listed in Table 30 along with their corresponding HMM bit scores.

An HMM was generated using the following sequences as input: SEQ ID NOs:162-164, SEQ ID NO:166, SEQ ID NO:169, and SEQ ID NO:171. The sequences are aligned in FIG. 5. When fitted to the HMM, the sequences had the HMM bit scores listed in Table 31. Other homologous and/or orthologous sequences also were fitted to the HMM, and these sequences are listed in Table 31 along with their corresponding HMM bit scores.

An HMM was generated using the following sequences as input: SEQ ID NOs:175-177, SEQ ID NO:179, and SEQ ID NOs:180-182. The sequences are aligned in FIG. 6. When fitted to the HMM, the sequences had the HMM bit scores listed in Table 32. Other homologous and/or orthologous sequences also were fitted to the HMM, and these sequences are listed in Table 32 along with their corresponding HMM bit scores.

An HMM was generated using the following sequences as input: SEQ ID NOs:185-187. The sequences are aligned in FIG. 7. When fitted to the HMM, the sequences had the HMM bit scores listed in Table 33. Another homologous and/or orthologous sequence, SEQ ID NO:188, also was fitted to the HMM, and the HMM bit score of this sequence is listed in Table 33.

An HMM was generated using the following sequences as input: SEQ ID NOs:190-193, SEQ ID NO:195, SEQ ID NO:196, SEQ ID NO:311, SEQ ID NO:315, and SEQ ID NO:317. The sequences are aligned in FIG. 8. When fitted to the HMM, the sequences had the HMM bit scores listed in Table 34. Another homologous and/or orthologous sequence, SEQ ID NO:313, also was fitted to the HMM, and the HMM bit score of this sequence is listed in Table 34.

An HMM was generated using the following sequences as input: SEQ ID NOs:198-199. The sequences are aligned in FIG. 9. When fitted to the HMM, the sequences had the HMM bit scores listed in Table 35.

An HMM was generated using the following sequences as input: SEQ ID NOs:203-204, SEQ ID NOs:206-208, SEQ ID NO:318, SEQ ID NO:320, and SEQ ID NO:321. The sequences are aligned in FIG. 10. When fitted to the HMM, the sequences had the HMM bit scores listed in Table 36. Other homologous and/or orthologous sequences also were fitted to the HMM, and these sequences are listed in Table 36 along with their corresponding HMM bit scores.

An HMM was generated using the following sequences as input: SEQ ID NO:216, SEQ ID NO:218, SEQ ID NOs:221-223, SEQ ID NOs:226-227, SEQ ID NO:323, SEQ ID NO:329, SEQ ID NOs:331-332, SEQ ID NOs:334-335, and SEQ ID NO:337. The sequences are aligned in FIG. 11. When fitted to the HMM, the sequences had the HMM bit scores listed in Table 37. Other homologous and/or orthologous sequences also were fitted to the HMM, and these sequences are listed in Table 37 along with their corresponding HMM bit scores.

An HMM was generated using the following sequences as input: SEQ ID NO:229, SEQ ID NO:230, SEQ ID NO:232, SEQ ID NO:234, SEQ ID NOs:238-239, SEQ ID NOs:241-242, SEQ ID NO:338, and SEQ ID NO:340. The sequences are aligned in FIG. 12. When fitted to the HMM, the sequences had the HMM bit scores listed in Table 38. Other homologous and/or orthologous sequences also were fitted to the HMM, and these sequences are listed in Table 38 along with their corresponding HMM bit scores.

An HMM was generated using the following sequences as input: SEQ ID NO:245 and SEQ ID NOs:247-248. The sequences are aligned in FIG. 13. When fitted to the HMM, the sequences had the HMM bit scores listed in Table 39.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-33. (canceled)

34. A plant cell comprising an exogenous nucleic acid, said exogenous nucleic acid comprising a nucleotide sequence encoding a polypeptide, wherein the HMM bit score of the amino acid sequence of said polypeptide is greater than 50, said HMM based on the amino acid sequences depicted in one of FIGS. 1-14, and wherein a tissue of a plant produced from said plant cell has a difference in the level of oil as compared to the corresponding level in tissue of a control plant that does not comprise said nucleic acid.

35. A plant cell comprising an exogenous nucleic acid, said exogenous nucleic acid comprising a nucleotide sequence encoding a polypeptide 50-85 amino acids in length, wherein said polypeptide is the amino terminus of a polypeptide having at least 450 amino acids and having an HMM bit score greater than 622, said HMM based on the amino acid sequences depicted in FIG. 15, and wherein a tissue of a plant produced from said plant cell has a difference in the level of oil as compared to the corresponding level in tissue of a control plant that does not comprise said nucleic acid.

36. A plant cell comprising an exogenous nucleic acid, said exogenous nucleic acid comprising a nucleotide sequence encoding a polypeptide having 80 percent or greater sequence identity to an amino acid sequence selected from the group consisting of SEQ ID NO:80, SEQ ID NOs:82-85, SEQ ID NOs:87-127, SEQ ID NOs:129-130, SEQ ID NOs:132-133, SEQ ID NOs:135-138, SEQ ID NOs:140-146, SEQ ID NOs:148-149, SEQ ID NO:151, SEQ ID NO:153, SEQ ID NOs:155-160, SEQ ID NOs:162-164, SEQ ID NO:166, SEQ ID NOs:168-171, SEQ ID NO:173, SEQ ID NOs:175-177, SEQ ID NOs:179-183, SEQ ID NOs:185-188, SEQ ID NOs:190-193, SEQ ID NOs:195-196, SEQ ID NOs:198-199, SEQ ID NO:201, SEQ ID NOs:203-214, SEQ ID NO:216, SEQ ID NO:218, SEQ ID NOs:220-227, SEQ ID NOs:229-230, SEQ ID NOs:232-235, SEQ ID NOs:237-243, SEQ ID NO:245, SEQ ID NOs:247-249, SEQ ID NO:309, SEQ ID NO:311, SEQ ID NO:313, SEQ ID NO:315, SEQ ID NOs:317-318, SEQ ID NOs:320-321, SEQ ID NO:323, SEQ ID NO:325, SEQ ID NO:327, SEQ ID NO:329, SEQ ID NOs:331-332, SEQ ID NOs:334-335, SEQ ID NOs:337-338, SEQ ID NOs:340-341, SEQ ID NOs:343-344, SEQ ID NO:346, SEQ ID NO:348, SEQ ID NO:350, SEQ ID NOs:352-353, SEQ ID NO:355, SEQ ID NO:357, SEQ ID NOs:359-360, SEQ ID NO:367, and SEQ ID NOs:415-441, wherein a tissue of a plant produced from said plant cell has a difference in the level of oil as compared to the corresponding level in tissue of a control plant that does not comprise said nucleic acid.

37. A plant cell comprising an exogenous nucleic acid, said exogenous nucleic acid comprising a nucleotide sequence having 80 percent or greater sequence identity to a nucleotide sequence selected from the group consisting of SEQ ID NO:79, SEQ ID NO:81, SEQ ID NO:86, SEQ ID NO:128, SEQ ID NO:131, SEQ ID NO:134, SEQ ID NO:139, SEQ ID NO:147, SEQ ID NO:150, SEQ ID NO:152, SEQ ID NO:154, SEQ ID NO:161, SEQ ID NO:165, SEQ ID NO:167, SEQ ID NO:172, SEQ ID NO:174, SEQ ID NO:178, SEQ ID NO:184, SEQ ID NO:189, SEQ ID NO:194, SEQ ID NO:197, SEQ ID NO:200, SEQ ID NO:202, SEQ ID NO:215, SEQ ID NO:217, SEQ ID NO:219, SEQ ID NO:228, SEQ ID NO:231, SEQ ID NO:236, SEQ ID NO:244, SEQ ID NO:246, SEQ ID NOs:265-308, SEQ ID NO:310, SEQ ID NO:312, SEQ ID NO:314, SEQ ID NO:316, SEQ ID NO:319, SEQ ID NO:322, SEQ ID NO:324, SEQ ID NO:326, SEQ ID NO:328, SEQ ID NO:330, SEQ ID NO:333, SEQ ID NO:336, SEQ ID NO:339, SEQ ID NO:342, SEQ ID NO:345, SEQ ID NO:347, SEQ ID NO:349, SEQ ID NO:351, SEQ ID NO:354, SEQ ID NO:356, SEQ ID NO:358, and SEQ ID NOs:361-366, wherein a tissue of a plant produced from said plant cell has a difference in the level of oil as compared to the corresponding level in tissue of a control plant that does not comprise said nucleic acid.

38. A plant cell comprising an exogenous nucleic acid, said exogenous nucleic acid comprising a regulatory region operably linked to a polynucleotide whose transcription product is at least 30 nucleotides in length and is complementary to a nucleic acid encoding a polypeptide, wherein the HMM bit score of the amino acid sequence of said polypeptide is greater than 50, said HMM based on the amino acid sequences depicted in one of FIGS. 1-14, wherein said regulatory region modulates transcription of said polynucleotide in said plant cell, and wherein a tissue of a plant produced from said plant cell has a difference in the level of oil as compared to the corresponding level in tissue of a control plant that does not comprise said nucleic acid.

39. A plant cell comprising an exogenous nucleic acid, said exogenous nucleic acid comprising a regulatory region operably linked to a polynucleotide that is transcribed into an interfering RNA effective for inhibiting expression of a polypeptide having 80 percent or greater sequence identity to an amino acid sequence selected from the group consisting of SEQ ID NO:80, SEQ ID NOs:82-85, SEQ ID NOs:87-127, SEQ ID NOs:129-130, SEQ ID NOs:132-133, SEQ ID NOs:135-138, SEQ ID NOs:140-146, SEQ ID NOs:148-149, SEQ ID NO:151, SEQ ID NO:153, SEQ ID NOs:155-160, SEQ ID NOs:162-164, SEQ ID NO:166, SEQ ID NOs:168-171, SEQ ID NO:173, SEQ ID NOs:175-177, SEQ ID NOs:179-183, SEQ ID NOs:185-188, SEQ ID NOs:190-193, SEQ ID NOs:195-196, SEQ ID NOs:198-199, SEQ ID NO:201, SEQ ID NOs:203-214, SEQ ID NO:216, SEQ ID NO:218, SEQ ID NOs:220-227, SEQ ID NOs:229-230, SEQ ID NOs:232-235, SEQ ID NOs:237-243, SEQ ID NO:245, SEQ ID NOs:247-249, SEQ ID NO:309, SEQ ID NO:311, SEQ ID NO:313, SEQ ID NO:315, SEQ ID NOs:317-318, SEQ ID NOs:320-321, SEQ ID NO:323, SEQ ID NO:325, SEQ ID NO:327, SEQ ID NO:329, SEQ ID NOs:331-332, SEQ ID NOs:334-335, SEQ ID NOs:337-338, SEQ ID NOs:340-341, SEQ ID NOs:343-344, SEQ ID NO:346, SEQ ID NO:348, SEQ ID NO:350, SEQ ID NOs:352-353, SEQ ID NO:355, SEQ ID NO:357, SEQ ID NOs:359-360, SEQ ID NO:367, and SEQ ID NOs:415-441, wherein said regulatory region modulates transcription of said polynucleotide in said plant cell, and wherein a tissue of a plant produced from said plant cell has a difference in the level of oil as compared to the corresponding level in tissue of a control plant that does not comprise said nucleic acid.

40. The plant cell of any of claims 34-39, wherein said plant is a dicot.

41. The plant cell of claim 40, wherein said plant is a member of the genus Anacardium, Arachis, Azadirachta, Brassica, Cannabis, Carthamus, Corylus, Crambe, Cucurbita, Glycine, Gossypium, Helianthus, Jatropha, Juglans, Linum, Olea, Papaver, Persea, Prunus, Ricinus, Sesamum, Simmondsia, or Vitis.

42. The plant cell of any of claims 34-39, wherein said plant is a monocot.

43. The plant cell of claim 42 wherein said plant is a member of the genus Cocos, Elaeis, Oryza, or Zea.

44. The plant cell of any of claims 34-39, wherein said plant is a species selected from the group consisting of Miscanthus hybrid (Miscanthus×giganteus), Miscanthus sinensis, Miscanthus sacchariflorus, Panicum virgatum, Populus balsamifera, Sorghum bicolor, and Saccharum spp.

45. The plant cell of any of claims 34-39, wherein said tissue is seed tissue.

46. A transgenic plant comprising the plant cell of any one of claims 34-39.

47. Progeny of the plant of claim 46, wherein said progeny has a difference in the level of oil as compared to the level of oil in a corresponding control plant that does not comprise said exogenous nucleic acid.

48. Seed from a transgenic plant according to claim 46.

49. Vegetative tissue from a transgenic plant according to claim 46.

50. Fruit from a transgenic plant according to claim 46.

51.-98. (canceled)
